SEQUENCE LISTING (289 sequences)

SEQ ID NO: 1 - DNA, 38 bp, Artificial Sequence
  Synthetic polynucleotide
gtacctttctattctcactctggtggaggcggtggatc

SEQ ID NO: 2 - DNA, 38 bp, Artificial Sequence
  Synthetic polynucleotide
ggccgatccaccgcctccaccagagtgagaatagaaag

SEQ ID NO: 3 - DNA, 15 bp, Artificial Sequence
  Synthetic polynucleotide
gctggcgggggaggg

SEQ ID NO: 4 - DNA, 23 bp, Artificial Sequence
  Synthetic polynucleotide
gatcccctcccccgccagctgca

SEQ ID NO: 5 - DNA, 58 bp, Artificial Sequence
  Synthetic polynucleotide
cggccatggcgggcggaggtggaggctacccatacgatgttccagattacgctcaggg

SEQ ID NO: 6 - DNA, 58 bp, Artificial Sequence
  Synthetic polynucleotide
tgagcgtaatctggaacatcgtatgggtagcctccacctccgcccgccatggccggct

SEQ ID NO: 7 - DNA, 45 bp, Artificial Sequence
  Synthetic polynucleotide
gttccgatgctgtctttcgctgctgcagattcgccgcatactgag

SEQ ID NO: 8 - DNA, 45 bp, Artificial Sequence
  Synthetic polynucleotide
ctcagtatgcggcgaatctgcagcagcgaaagacagcatcggaac

SEQ ID NO: 9 - protein, 6 aa, Artificial Sequence
  Synthetic polypeptide
AAGGGG

SEQ ID NO: 10 - protein, 5 aa, Artificial Sequence
  Synthetic polypeptide
LPETG

SEQ ID NO: 11 - protein, 5 aa, Artificial Sequence
  Synthetic polypeptide
LPETA

SEQ ID NO: 12 - protein, 6 aa, Artificial Sequence
  Synthetic polypeptide
LPETAA

SEQ ID NO: 13 - protein, 6 aa, Artificial Sequence
  Synthetic polypeptide
LPETGG

SEQ ID NO: 14 - protein, 655 aa, Artificial Sequence
  Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLPETGGGGGSAETVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDLFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 15 - protein, 296 aa, Artificial Sequence
  Synthetic polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKLPETGGGGGYPYDVPDYAQGGQGVDMSVLVYSFASFVLGWCLRSGITYFTRLMETSS

SEQ ID NO: 16 - protein, 295 aa, Artificial Sequence
  Synthetic polypeptide
MVSKGEELFTGVVPILVELDGDVNGHKESVSGEGEGDATYGKLTLKFICTTGKLPVPWPTLVTTLTYGVQCFSRYPDHMKQHDFFKSATPEGYVQQDPTIFCKDDGNYKTRAEVKFEGDTLVNRIELKGIDFKEDGNILGHKLEYNYNSHNVYIMADKQKNGTKVNFKTRHNTEDGSVQLADHYQQNTPIGDGPVLLPDNHYLSTQSALSKDPNEKRDHMVLLEFVTAAGITLGMDELYKLPETAAGGGGDPAKAAFNSLQASATEYIGYAWAMVVVTVGATTGTKLFKKFTSAS

SEQ ID NO: 17 - protein, 546 aa, Artificial Sequence
  Synthetic polypeptide
MAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASLELPETGGGGGSAETVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDLFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 18 - protein, 551 aa, Artificial Sequence
  Synthetic polypeptide
EPWIHHAPPGCGNALPETGGGTPQNITDLCAEYHNTQIHTLNDKIFSYTESLAGKREMAIITFKNGATFQVEVPGSQHIDSQKKAIERMKDTLRIAYLTEAKVEKLCVWNNKTPHAIAAISMANSSMSNTCDEKTQSLGVKGGGSAETVESCLAKSHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDLFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFGAGKPYEFSIDCDKINLFRGVFAFLLYVATFMYVFSTFANILRNKES

SEQ ID NO: 19 - protein, 5 aa, Artificial Sequence
  Synthetic polypeptide
GGGGS

SEQ ID NO: 20 - protein, 10 aa, Artificial Sequence
  Synthetic polypeptide
GGGGSGGGGS

SEQ ID NO: 21 - protein, 5 aa, Artificial Sequence
  Synthetic polypeptide
LPKTG

SEQ ID NO: 22 - protein, 5 aa, Artificial Sequence
  Synthetic polypeptide
LPATG

SEQ ID NO: 23 - protein, 5 aa, Artificial Sequence
  Synthetic polypeptide
LPNTG

SEQ ID NO: 24 - protein, 5 aa, Artificial Sequence
  Synthetic polypeptide
LPXAG

SEQ ID NO: 25 - protein, 5 aa, Artificial Sequence
  Synthetic polypeptide
LPNAG

SEQ ID NO: 26 - protein, 5 aa, Artificial Sequence
  Synthetic polypeptide
LPXTA

SEQ ID NO: 27 - protein, 5 aa, Artificial Sequence
  Synthetic polypeptide
LPNTA

SEQ ID NO: 28 - protein, 5 aa, Artificial Sequence
  Synthetic polypeptide
LGXTG

SEQ ID NO: 29 - protein, 5 aa, Artificial Sequence
  Synthetic polypeptide
LGATG

SEQ ID NO: 30 - protein, 5 aa, Artificial Sequence
  Synthetic polypeptide
IPXTG

SEQ ID NO: 31 - protein, 5 aa, Artificial Sequence
  Synthetic polypeptide
IPNTG

SEQ ID NO: 32 - protein, 5 aa, Artificial Sequence
  Synthetic polypeptide
IPETG

SEQ ID NO: 33 - protein, 5 aa, Artificial Sequence
  Synthetic polypeptide
NPQTN

SEQ ID NO: 34 - protein, 5 aa, Artificial Sequence
  Synthetic polypeptide
NPKTG

SEQ ID NO: 35 - protein, 5 aa, Artificial Sequence
  Synthetic polypeptide
NPQTN

SEQ ID NO: 36 - protein, 5 aa, Artificial Sequence
  Synthetic polypeptide
NPKTG

SEQ ID NO: 37 - protein, 5 aa, Artificial Sequence
  Synthetic polypeptide
NSKTA

SEQ ID NO: 38 - protein, 5 aa, Artificial Sequence
  Synthetic polypeptide
NPQTG

SEQ ID NO: 39 - protein, 5 aa, Artificial Sequence
  Synthetic polypeptide
NAKTN

SEQ ID NO: 40 - protein, 5 aa, Artificial Sequence
  Synthetic polypeptide
NPQSS

SEQ ID NO: 41 - protein, 5 aa, Artificial Sequence
  Synthetic polypeptide
NAKTN

SEQ ID NO: 42 - protein, 5 aa, Artificial Sequence
  Synthetic polypeptide
NPQSS

SEQ ID NO: 43 - protein, 5 aa, Artificial Sequence
  Synthetic polypeptide
LPXTA

SEQ ID NO: 44 - protein, 5 aa, Artificial Sequence
  Synthetic polypeptide
LAXTG

SEQ ID NO: 45 - protein, 5 aa, Artificial Sequence
  Synthetic polypeptide
LPXTA

SEQ ID NO: 46 - protein, 5 aa, Artificial Sequence
  Synthetic polypeptide
LAXTG

SEQ ID NO: 47 - protein, 5 aa, Artificial Sequence
  Synthetic polypeptide
LPNTA

SEQ ID NO: 48 - protein, 6 aa, Artificial Sequence
  Synthetic polypeptide
QVPTGV

SEQ ID NO: 49 - protein, 5 aa, Artificial Sequence
  Synthetic polypeptide
LPXTA

SEQ ID NO: 50 - protein, 5 aa, Artificial Sequence
  Synthetic polypeptide
LPSTS

SEQ ID NO: 51 - protein, 5 aa, Artificial Sequence
  Synthetic polypeptide
LPKTG

SEQ ID NO: 52 - protein, 5 aa, Artificial Sequence
  Synthetic polypeptide
LPITG

SEQ ID NO: 53 - protein, 5 aa, Artificial Sequence
  Synthetic polypeptide
LPDTA

SEQ ID NO: 54 - protein, 5 aa, Artificial Sequence
  Synthetic polypeptide
SPKTG

SEQ ID NO: 55 - protein, 5 aa, Artificial Sequence
  Synthetic polypeptide
LAETG

SEQ ID NO: 56 - protein, 5 aa, Artificial Sequence
  Synthetic polypeptide
LAATG

SEQ ID NO: 57 - protein, 5 aa, Artificial Sequence
  Synthetic polypeptide
LAHTG

SEQ ID NO: 58 - protein, 5 aa, Artificial Sequence
  Synthetic polypeptide
LASTG

SEQ ID NO: 59 - protein, 5 aa, Artificial Sequence
  Synthetic polypeptide
LAETG

SEQ ID NO: 60 - protein, 5 aa, Artificial Sequence
  Synthetic polypeptide
LPLTG

SEQ ID NO: 61 - protein, 5 aa, Artificial Sequence
  Synthetic polypeptide
LSRTG

SEQ ID NO: 62 - protein, 5 aa, Artificial Sequence
  Synthetic polypeptide
VPDTG

SEQ ID NO: 63 - protein, 5 aa, Artificial Sequence
  Synthetic polypeptide
IPQTG

SEQ ID NO: 64 - protein, 5 aa, Artificial Sequence
  Synthetic polypeptide
YPRRG

SEQ ID NO: 65 - protein, 5 aa, Artificial Sequence
  Synthetic polypeptide
LPMTG

SEQ ID NO: 66 - protein, 5 aa, Artificial Sequence
  Synthetic polypeptide
LPLTG

SEQ ID NO: 67 - protein, 5 aa, Artificial Sequence
  Synthetic polypeptide
LAFTG

SEQ ID NO: 68 - protein, 5 aa, Artificial Sequence
  Synthetic polypeptide
LPQTS

SEQ ID NO: 69 - protein, 45 aa, Artificial Sequence
  Synthetic polypeptide
TTTTTTTTTTCTGCAGACGTCTTACCTCTAATCGATCGATCTCCG

SEQ ID NO: 70 - protein, 4 aa, Artificial Sequence
  Synthetic polypeptide
LPST

SEQ ID NO: 71 - protein, 4 aa, Artificial Sequence
  Synthetic polypeptide
NSKT

SEQ ID NO: 72 - protein, 4 aa, Artificial Sequence
  Synthetic polypeptide
NPQT

SEQ ID NO: 73 - protein, 4 aa, Artificial Sequence
  Synthetic polypeptide
NAKT

SEQ ID NO: 74 - protein, 4 aa, Artificial Sequence
  Synthetic polypeptide
LPIT

SEQ ID NO: 75 - protein, 4 aa, Artificial Sequence
  Synthetic polypeptide
LAET

SEQ ID NO: 76 - protein, 4 aa, Artificial Sequence
  Synthetic polypeptide
NPQS

SEQ ID NO: 77 - protein, 5 aa, Artificial Sequence
  Synthetic polypeptide
GGGGG

SEQ ID NO: 78 - protein, 5 aa, Artificial Sequence
  Synthetic polypeptide
LPXTG

SEQ ID NO: 79 - DNA, 6407 bp, Artificial Sequence
  Synthetic polynucleotide
aacgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttacatggaatgaaacttccagacaccgtactttagttgcatatttaaaacatgttgagctacagcaccagattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattctttcgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagcctttttttttggagattttcaacatgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggttgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttagcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgctaataattttgatatggttggttcaattccttccataattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgcacctaaagatattttagataaccttcctcaattcctttctactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaagattctggcgtaccgttcctgtctaaaatccctttaatcggcctcctgtttagctcccgctctgattccaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagacctctcaaaaatagctaccctctccggcatgaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 80 - protein, 410 aa, Artificial Sequence
  Synthetic polypeptide
MIDMLVLRLPFIDSLVCSRLSGNDLIAFVDLSKIATLSGMNLSARTVEYHIDGDLTVSGLSHPFESLPTHYSGIAFKIYEGSKNFYPCVEIKASPAKVLQGHNVFGTTDLALCSEALLLNFANSLPCLYDLLDVNATTISRIDATFSARAPNENIAKQVIDHLRNVSNGQTKSTRSQNWESTVTWNETSRHRTLVAYLKHVELQHQIQQLSSKPSAKMTSYQKEQLKVLSNPDLLEFASGLVRFEARIKTRYLKSFGLPLNLFDAIRFASDYNSQGKDLIFDLWSFSFSELFKAFEGDSMNIYDDSAVLDAIQSKHFTITPSGKTSFAKASRYFGFYRRLVNEGYDSVALTMPRNSFWRYVSALVECGIPKSQLMNLSTCNNVVPLVRFINVDFSSQRPDWYNEPVLKIA

SEQ ID NO: 81 - protein, 110 aa, Artificial Sequence
  Synthetic polypeptide
MNIYDDSAVLDAIQSKHFTITPSGKTSFAKASRYFGFYRRLVNEGYDSVALTMPRNSFWRYVSALVECGIPKSQLMNLSTCNNVVPLVRFINVDFSSQRPDWYNEPVLKI

SEQ ID NO: 82 - protein, 87 aa, Artificial Sequence
  Synthetic polypeptide
MIKVEIKPSQAQFTTRSGVSRQGKPYSLNEQLCYVDLGNEYPVLVKITLDEGQPAYAPGLYTVHLSSFKVGQFGSLMIDRLRLVPAK

SEQ ID NO: 83 - protein, 33 aa, Artificial Sequence
  Synthetic polypeptide
MEQVADFDTIYQAMIQISVVLCFALGIIAGGQR

SEQ ID NO: 84 - protein, 32 aa, Artificial Sequence
  Synthetic polypeptide
MSVLVYSFASFVLGWCLRSGITYFTRLMETSS

SEQ ID NO: 85 - protein, 73 aa, Artificial Sequence
  Synthetic polypeptide
MKKSLVLKASVAVATLVPMLSFAAEGDDPAKAAFNSLQASATEYIGYAWAMVVVIVGATIGIKLFKKFTSKAS

SEQ ID NO: 86 - protein, 424 aa, Artificial Sequence
  Synthetic polypeptide
MKKLLFAIPLVVPFYSHSAETVESCLAKPHTENSFTNVWKDDKTLDRYANYEGCLWNATGVVVCTGDETQCYGTWVPIGLAIPENEGGGSEGGGSEGGGSEGGGTKPPEYGDTPIPGYTYINPLDGTYPPGTEQNPANPNPSLEESQPLNTFMFQNNRFRNRQGALTVYTGTVTQGTDPVKTYYQYTPVSSKAMYDAYWNGKFRDCAFHSGFNEDPFVCEYQGQSSDLPQPPVNAGGGSGGGSGGGSEGGGSEGGGSEGGGSEGGGSGGGSGSGDFDYEKMANANKGAMTENADENALQSDAKGKLDSVATDYGAAIDGFIGDVSGLANGNGATGDFAGSNSQMAQVGDGDNSPLMNNFRQYLPSLPQSVECRPFVFSAGKPYEFSIDCDKINLFRGVFAFLLYVATFMYVFSTFANILRNKES

SEQ ID NO: 87 - protein, 112 aa, Artificial Sequence
  Synthetic polypeptide
MPVLLGIPLLLRFLGFLLVTLFGYLLTFLKKGFGKIAIAISLFLALIIGLNSILVGYLSDISAQLPSDFVQGVQLILPSNALPCFYVILSVKAAIFIFDVKQKIVSYLDWDK

SEQ ID NO: 88 - protein, 348 aa, Artificial Sequence
  Synthetic polypeptide
MAVYFVTGKLGSGKTLVSVGKIQDKIVAGCKIATNLDLRLQNLPQVGRFAKTPRVLRIPDKPSISDLLAIGRGNDSYDENKNGLLVLDECGTWFNTRSWNDKERQPIIDWFLHARKLGWDIIFLVQDLSIVDKQARSALAEHVVYCRRLDRITLPFVGTLYSLITGSKMPLPKLHVGVVKYGDSQLSPTVERWLYTGKNLYNAYDTKQAFSSNYDSGVYSYLTPYLSHGRYFKPLNLGQKMKLTKIYLKKFSRVLCLAIGFASAFTYSYITQPKPEVKKVVSQTYDFDKFTIDSSQRLNLSYRYVFKDSKGKLINSDDLQKQGYSLTYIDLCTVSIKKGNSNEIVKCN

SEQ ID NO: 89 - protein, 426 aa, Artificial Sequence
  Synthetic polypeptide
MKLLNVINFVFLMFVSSSSFAQVIEMNNSPLRDFVTWYSKQSGESVIVSPDVKGTVTVYSSDVKPENLRNFFISVLRANNFDMVGSIPSIIQKYNPNNQDYIDELPSSDNQEYDDNSAPSGGFFVPQNDNVTQTFKINNVRAKDLIRVVELFVKSNTSKSSNVLSIDGSNLLVVSAPKDILDNLPQFLSTVDLPTDQILIEGLIFEVQQGDALDFSFAAGSQRGTVAGGVNTDRLTSVLSSAGGSFGIFNGDVLGLSVRALKTNSHSKILSVPRILTLSGQKGSISVGQNVPFITGRVTGESANVNNPFQTIERQNVGISMSVFPVAMAGGNIVLDITSKADSLSSSTQASDVITNQRSIATTVNLRDGQTLLLGGLTDYKNTSQDSGVPFLSKIPLIGLLFSSRSDSNEESTLYVLVKATIVRAL

SEQ ID NO: 90 - protein, 18 aa, Artificial Sequence
  Synthetic polypeptide
GGGSGGGSGGGSLPETGG

SEQ ID NO: 91 - protein, 5 aa, Artificial Sequence
  Synthetic polypeptide
LPXTA

SEQ ID NO: 92 - protein, 9 aa, Artificial Sequence
  Synthetic polypeptide
LPXTGGGGG

SEQ ID NO: 93 - protein, 4 aa, Artificial Sequence
  Synthetic polypeptide
LPKT

SEQ ID NO: 94 - protein, 4 aa, Artificial Sequence
  Synthetic polypeptide
LPIT

SEQ ID NO: 95 - protein, 4 aa, Artificial Sequence
  Synthetic polypeptide
LPDT

SEQ ID NO: 96 - protein, 4 aa, Artificial Sequence
  Synthetic polypeptide
SPKT

SEQ ID NO: 97 - protein, 4 aa, Artificial Sequence
  Synthetic polypeptide
LAET

SEQ ID NO: 98 - protein, 4 aa, Artificial Sequence
  Synthetic polypeptide
LAAT

SEQ ID NO: 99 - protein, 4 aa, Artificial Sequence
  Synthetic polypeptide
LAET

SEQ ID NO: 100 - protein, 4 aa, Artificial Sequence
  Synthetic polypeptide
LAST

SEQ ID NO: 101 - protein, 4 aa, Artificial Sequence
  Synthetic polypeptide
LAET

SEQ ID NO: 102 - protein, 4 aa, Artificial Sequence
  Synthetic polypeptide
LPLT

SEQ ID NO: 103 - protein, 4 aa, Artificial Sequence
  Synthetic polypeptide
LSRT

SEQ ID NO: 104 - protein, 4 aa, Artificial Sequence
  Synthetic polypeptide
LPET

SEQ ID NO: 105 - protein, 4 aa, Artificial Sequence
  Synthetic polypeptide
VPDT

SEQ ID NO: 106 - protein, 4 aa, Artificial Sequence
  Synthetic polypeptide
IPQT

SEQ ID NO: 107 - protein, 4 aa, Artificial Sequence
  Synthetic polypeptide
YPRR

SEQ ID NO: 108 - protein, 4 aa, Artificial Sequence
  Synthetic polypeptide
LPMT

SEQ ID NO: 109 - protein, 4 aa, Artificial Sequence
  Synthetic polypeptide
LPLT

SEQ ID NO: 110 - protein, 4 aa, Artificial Sequence
  Synthetic polypeptide
LAFT

SEQ ID NO: 111 - protein, 4 aa, Artificial Sequence
  Synthetic polypeptide
LPQT

SEQ ID NO: 112 - protein, 4 aa, Artificial Sequence
  Synthetic polypeptide
NSKT

SEQ ID NO: 113 - protein, 4 aa, Artificial Sequence
  Synthetic polypeptide
NPQT

SEQ ID NO: 114 - protein, 4 aa, Artificial Sequence
  Synthetic polypeptide
NAKT

SEQ ID NO: 115 - protein, 4 aa, Artificial Sequence
  Synthetic polypeptide
NPQS

SEQ ID NO: 116 - protein, 8 aa, Artificial Sequence
  Synthetic polypeptide
DSPHTELP

SEQ ID NO: 117 - protein, 15 aa, Artificial Sequence
  Synthetic polypeptide
GLQDIFEAQKIEWHE

SEQ ID NO: 118 - protein, 15 aa, Artificial Sequence
  Synthetic polypeptide
GLQDIFEAQKIEWHE

SEQ ID NO: 119 - protein, 8 aa, Artificial Sequence
  Synthetic polypeptide
DSPHTELP

SEQ ID NO: 120 - protein, 10 aa, Artificial Sequence
  Synthetic polypeptide
SPPTGINGGG

SEQ ID NO: 121 - DNA, 30 bp, Artificial Sequence
  Synthetic polynucleotide
acgtatcgtaggctcgcatctttttttttt

SEQ ID NO: 122 - DNA, 30 bp, Artificial Sequence
  Synthetic polynucleotide
ttttttttttctgcagttgaaccggtagca

SEQ ID NO: 123 - DNA, 30 bp, Artificial Sequence
  Synthetic polynucleotide
gagcctacgatacgttgctaccggttcaac

SEQ ID NO: 124 - DNA, 45 bp, Artificial Sequence
  Synthetic polynucleotide
gagcgtgattcggatccgtcattcatctacgcatctttttttttt

SEQ ID NO: 125 - DNA, 60 bp, Artificial Sequence
  Synthetic polynucleotide
gtagatgaatgacggatccgaatcacgctccggagatcgatcgattagaggtaagacgtc

SEQ ID NO: 126 - protein, 6 aa, Artificial Sequence
  Synthetic polypeptide
LPXTAA

SEQ ID NO: 127 - protein, 4 aa, Artificial Sequence
  Synthetic polypeptide
GGGK

SEQ ID NO: 128 - DNA, 185 bp, Artificial Sequence
  Synthetic polynucleotide
gtacctttctattctcactctgagccgtggattcatcatgcaccgccgggttgtgggaatgctcttcctgagaccggtggttacccatacgatgttccagattacgctatgaatgctccaagatcatcgatgagtaatacttgcgatgaaaaaacccaaagtctaggtgtaaaaggaggcgggtc

SEQ ID NO: 129 - DNA, 185 bp, Artificial Sequence
  Synthetic polynucleotide
ggccgacccgcctccttttacacctagactttgggttttttcatcgcaagtattactcatcgatgatcttggagcattcatagcgtaatctggaacatcgtatgggtaaccaccggtctcaggaagagcattcccacaacccggcggtgcatgatgaatccacggctcagagtgagaatagaaag

SEQ ID NO: 130 - DNA, 41 bp, Artificial Sequence
  Synthetic polynucleotide
gttccagattacgctattgaagggagatcatcgatgaatac

SEQ ID NO: 131 - DNA, 41 bp, Artificial Sequence
  Synthetic polynucleotide
gtattcatcgatgatctcccttcaatagcgtaatctggaac

SEQ ID NO: 132 - DNA, 24 bp, Artificial Sequence
  Synthetic polynucleotide
gcttatgatacgaatatggattcg

SEQ ID NO: 133 - DNA, 32 bp, Artificial Sequence
  Synthetic polynucleotide
gatccgaatccatattcgtatcataagctgca

SEQ ID NO: 134 - DNA, 53 bp, Artificial Sequence
  Synthetic polynucleotide
cgtttgctaacatactccggaataaggagtcttgatcatgccagttcttttgg

SEQ ID NO: 135 - DNA, 53 bp, Artificial Sequence
  Synthetic polynucleotide
ccaaaagaactggcatgatcaagactccttattccggagtatgttagcaaacg

SEQ ID NO: 136 - DNA, 46 bp, Artificial Sequence
  Synthetic polynucleotide
aggctgctattttcatttttgacgtcaaacaaaaaatcgtttctta

SEQ ID NO: 137 - DNA, 46 bp, Artificial Sequence
  Synthetic polynucleotide
taagaaacgattttttgtttgacgtcaaaaatgaaaatagcagcct

SEQ ID NO: 138 - DNA, 46 bp, Artificial Sequence
  Synthetic polynucleotide
atatggctgtttattttgtaaccggtaaattaggctctggaaagac

SEQ ID NO: 139 - DNA, 46 bp, Artificial Sequence
  Synthetic polynucleotide
gtctttccagagcctaatttaccggttacaaaataaacagccatat

SEQ ID NO: 140 - DNA, 48 bp, Artificial Sequence
  Synthetic polynucleotide
tattttacccgtttaatggaaactagttcatgaaaaagtctttagtcc

SEQ ID NO: 141 - DNA, 48 bp, Artificial Sequence
  Synthetic polynucleotide
ggactaaagactttttcatgaactagtttccattaaacgggtaaaata

SEQ ID NO: 142 - DNA, 62 bp, Artificial Sequence
  Synthetic polynucleotide
ccggaataaggagtctctaccggaaacaggaggctacccatacgatgttccagattacgctt

SEQ ID NO: 143 - DNA, 62 bp, Artificial Sequence
  Synthetic polynucleotide
gatcaagcgtaatctggaacatcgtatgggtagcctcctgtttccggtagagactccttatt

SEQ ID NO: 144 - DNA, 74 bp, Artificial Sequence
  Synthetic polynucleotide
ccggaataaggagtctggaggtggaagtctaccggaaacaggaggctacccatacgatgttccagattacgctt

SEQ ID NO: 145 - DNA, 74 bp, Artificial Sequence
  Synthetic polynucleotide
gatcaagcgtaatctggaacatcgtatgggtagcctcctgtttccggtagacttccacctccagactccttatt

SEQ ID NO: 146 - DNA, 98 bp, Artificial Sequence
  Synthetic polynucleotide
ccggaataaggagtctggaggtggaagtggcggtgggagcgggggaggctctctaccggaaacaggaggctacccatacgatgttccagattacgctt

SEQ ID NO: 147 - DNA, 98 bp, Artificial Sequence
  Synthetic polynucleotide
gatcaagcgtaatctggaacatcgtatgggtagcctcctgtttccggtagagagcctcccccgctcccaccgccacttccacctccagactccttatt

SEQ ID NO: 148 - DNA, 105 bp, Artificial Sequence
  Synthetic polynucleotide
caaacaaaaaatcgtttcttatttggattgggataaactaccggaaacaggaggctacccatacgacgttccagattacgcttaatatggctgtttattttgtaa

SEQ ID NO: 149 - DNA, 113 bp, Artificial Sequence
  Synthetic polynucleotide
ccggttacaaaataaacagccatattaagcgtaatctggaacgtcgtatgggtagcctcctgtttccggtagtttatcccaatccaaataagaaacgattttttgtttgacgt

SEQ ID NO: 150 - DNA, 117 bp, Artificial Sequence
  Synthetic polynucleotide
caaacaaaaaatcgtttcttatttggattgggataaaggaggtggaagtctaccggaaacaggaggctacccatacgacgttccagattacgcttaatatggctgtttattttgtaa

SEQ ID NO: 151 - DNA, 125 bp, Artificial Sequence
  Synthetic polynucleotide
ccggttacaaaataaacagccatattaagcgtaatctggaacgtcgtatgggtagcctcctgtttccggtagacttccacctcctttatcccaatccaaataagaaacgattttttgtttgacgt

SEQ ID NO: 152 - DNA, 141 bp, Artificial Sequence
  Synthetic polynucleotide
caaacaaaaaatcgtttcttatttggattgggataaaggaggtggaagtggcggtgggagcgggggaggctctctaccggaaacaggaggctacccatacgacgttccagattacgcttaatatggctgtttattttgtaa

SEQ ID NO: 153 - DNA, 149 bp, Artificial Sequence
  Synthetic polynucleotide
ccggttacaaaataaacagccatattaagcgtaatctggaacgtcgtatgggtagcctcctgtttccggtagagagcctcccccgctcccaccgccacttccacctcctttatcccaatccaaataagaaacgattttttgtttgacgt

SEQ ID NO: 154 - DNA, 54 bp, Artificial Sequence
  Synthetic polynucleotide
ctagttctctcccggaaacaggtggatacccatacgatgttccagattacgctt

SEQ ID NO: 155 - DNA, 54 bp, Artificial Sequence
  Synthetic polynucleotide
catgaagcgtaatctggaacatcgtatgggtatccacctgtttccgggagagaa

SEQ ID NO: 156 - DNA, 66 bp, Artificial Sequence
  Synthetic polynucleotide
ctagttctggaggtggaagtctcccggaaacaggtggatacccatacgatgttccagattacgctt

SEQ ID NO: 157 - DNA, 66 bp, Artificial Sequence
  Synthetic polynucleotide
catgaagcgtaatctggaacatcgtatgggtatccacctgtttccgggagacttccacctccagaa

SEQ ID NO: 158 - DNA, 90 bp, Artificial Sequence
  Synthetic polynucleotide
ctagttctggaggtggaagtggcggtgggagcgggggaggctctctcccggaaacaggtggatacccatacgatgttccagattacgctt

SEQ ID NO: 159 - DNA, 90 bp, Artificial Sequence
  Synthetic polynucleotide
catgaagcgtaatctggaacatcgtatgggtatccacctgtttccgggagagagcctcccccgctcccaccgccacttccacctccagaa

SEQ ID NO: 160 - DNA, 20 bp, Artificial Sequence
  Synthetic polynucleotide
ccctcatagttagcgtaacg

SEQ ID NO: 161 - DNA, 20 bp, Artificial Sequence
  Synthetic polynucleotide
gttgctattttgcacccagc

SEQ ID NO: 162 - protein, 27 aa, Artificial Sequence
  Synthetic polypeptide
MVSKGEELFTGVVPILVELDGDVNGHK

SEQ ID NO: 163 - protein, 23 aa, Artificial Sequence
  Synthetic polypeptide
GEELFTGVVPILVELDGDVNGHK

SEQ ID NO: 164 - protein, 15 aa, Artificial Sequence
  Synthetic polypeptide
FSVSGEGEGDATYGK

SEQ ID NO: 165 - protein, 19 aa, Artificial Sequence
  Synthetic polypeptide
FSVSGEGEGDATYGKLTLK

SEQ ID NO: 166 - protein, 7 aa, Artificial Sequence
  Synthetic polypeptide
FICTTGK

SEQ ID NO: 167 - protein, 27 aa, Artificial Sequence
  Synthetic polypeptide
LPVPWPTLVTTLTYGVQCFSRYPDHMK

SEQ ID NO: 168 - protein, 12 aa, Artificial Sequence
  Synthetic polypeptide
YPDHMKQHDFFK

SEQ ID NO: 169 - protein, 23 aa, Artificial Sequence
  Synthetic polypeptide
YPDHMKQHDFFKSAMPEGYVQER

SEQ ID NO: 170 - protein, 6 aa, Artificial Sequence
  Synthetic polypeptide
QHDFFK

SEQ ID NO: 171 - protein, 17 aa, Artificial Sequence
  Synthetic polypeptide
QHDFFKSAMPEGYVQER

SEQ ID NO: 172 - protein, 22 aa, Artificial Sequence
  Synthetic polypeptide
QHDFFKSAMPEGYVQERTIFFK

SEQ ID NO: 173 - protein, 11 aa, Artificial Sequence
  Synthetic polypeptide
SAMPEGYVQER

SEQ ID NO: 174 - protein, 16 aa, Artificial Sequence
  Synthetic polypeptide
SAMPEGYVQERTIFFK

SEQ ID NO: 175 - protein, 22 aa, Artificial Sequence
  Synthetic polypeptide
SAMPEGYVQERTIFFKDDGNYK

SEQ ID NO: 176 - protein, 11 aa, Artificial Sequence
  Synthetic polypeptide
TIFFKDDGNYK

SEQ ID NO: 177 - protein, 13 aa, Artificial Sequence
  Synthetic polypeptide
TIFFKDDGNYKTR

SEQ ID NO: 178 - protein, 12 aa, Artificial Sequence
  Synthetic polypeptide
DDGNYKTRAEVK

SEQ ID NO: 179 - protein, 15 aa, Artificial Sequence
  Synthetic polypeptide
TRAEVKFEGDTLVNR

SEQ ID NO: 180 - protein, 13 aa, Artificial Sequence
  Synthetic polypeptide
AEVKFEGDTLVNR

SEQ ID NO: 181 - protein, 9 aa, Artificial Sequence
  Synthetic polypeptide
FEGDTLVNR

SEQ ID NO: 182 - protein, 13 aa, Artificial Sequence
  Synthetic polypeptide
FEGDTLVNRIELK

SEQ ID NO: 183 - protein, 9 aa, Artificial Sequence
  Synthetic polypeptide
IELKGIDFK

SEQ ID NO: 184 - protein, 18 aa, Artificial Sequence
  Synthetic polypeptide
IELKGIDFKEDGNILGHK

SEQ ID NO: 185 - protein, 14 aa, Artificial Sequence
  Synthetic polypeptide
GIDFKEDGNILGHK

SEQ ID NO: 186 - protein, 9 aa, Artificial Sequence
  Synthetic polypeptide
EDGNILGHK

SEQ ID NO: 187 - protein, 25 aa, Artificial Sequence
  Synthetic polypeptide
EDGNILGHKLEYNYNSHNVYIMADK

SEQ ID NO: 188 - protein, 16 aa, Artificial Sequence
  Synthetic polypeptide
LEYNYNSHNVYIMADK

SEQ ID NO: 189 - protein, 17 aa, Artificial Sequence
  Synthetic polypeptide
LEYNYNSHVYIMADKQK

SEQ ID NO: 190 - protein, 22 aa, Artificial Sequence
  Synthetic polypeptide
LEYNYNSHNVYIMADKQKNGIK

SEQ ID NO: 191 - protein, 6 aa, Artificial Sequence
  Synthetic polypeptide
QKNGIK

SEQ ID NO: 192 - protein, 8 aa, Artificial Sequence
  Synthetic polypeptide
NGIKVNFK

SEQ ID NO: 193 - protein, 10 aa, Artificial Sequence
  Synthetic polypeptide
NGIKVNFKIR

SEQ ID NO: 194 - protein, 24 aa, Artificial Sequence
  Synthetic polypeptide
RDHMVLLEFVTAAGITLGMDELYK

SEQ ID NO: 195 - protein, 23 aa, Artificial Sequence
  Synthetic polypeptide
DHMVLLEFVTAAGITLGMDELYK

SEQ ID NO: 196 - protein, 20 aa, Artificial Sequence
  Synthetic polypeptide
LPETGGGGGSAETVESCLAK

SEQ ID NO: 197 - protein, 12 aa, Artificial Sequence
  Synthetic polypeptide
SHTENSFTNVWK

SEQ ID NO: 198 - protein, 15 aa, Artificial Sequence
  Synthetic polypeptide
SHTENSFTNVWKDDK

SEQ ID NO: 199 - protein, 7 aa, Artificial Sequence
  Synthetic polypeptide
DDKTLDR

SEQ ID NO: 200 - protein, 23 aa, Artificial Sequence
  Synthetic polypeptide
FRNRQGALTVYTGTVTQGTDPVK

SEQ ID NO: 201 - protein, 19 aa, Artificial Sequence
  Synthetic polypeptide
QGALTVYTGTVTQGTDPVK

SEQ ID NO: 202 - protein, 11 aa, Artificial Sequence
  Synthetic polypeptide
TYYQYTPVSSK

SEQ ID NO: 203 - protein, 23 aa, Artificial Sequence
  Synthetic polypeptide
TYYQYTPVSSKAMYDAYWNGKFR

SEQ ID NO: 204 - protein, 10 aa, Artificial Sequence
  Synthetic polypeptide
AMYDAYWNGK

SEQ ID NO: 205 - protein, 6 aa, Artificial Sequence
  Synthetic polypeptide
MANANK

SEQ ID NO: 206 - protein, 25 aa, Artificial Sequence
  Synthetic polypeptide
MANANKGAMTENADENALQSDAKGK

SEQ ID NO: 207 - protein, 17 aa, Artificial Sequence
  Synthetic polypeptide
GAMTENADENALQSDAK

SEQ ID NO: 208 - protein, 24 aa, Artificial Sequence
  Synthetic polypeptide
GVFAFLLYVATFMYVFSTFANILR

SEQ ID NO: 209 - protein, 26 aa, Artificial Sequence
  Synthetic polypeptide
GVFAFLLYVATFMYVFSTFANILRNK

SEQ ID NO: 210 - protein, 19 aa, Artificial Sequence
  Synthetic polypeptide
MVSKGEELFTGVVPILVEL

SEQ ID NO: 211 - protein, 21 aa, Artificial Sequence
  Synthetic polypeptide
MVSKGEELFTGVVPILVELDG

SEQ ID NO: 212 - protein, 13 aa, Artificial Sequence
  Synthetic polypeptide
ELFTGVVPILVEL

SEQ ID NO: 213 - protein, 17 aa, Artificial Sequence
  Synthetic polypeptide
DGDVNGHKFSVSGEGEG

SEQ ID NO: 214 - protein, 15 aa, Artificial Sequence
  Synthetic polypeptide
DVNGHKFSVSGEGEG

SEQ ID NO: 215 - protein, 16 aa, Artificial Sequence
  Synthetic polypeptide
EGEGDATYGKLTLKFI

SEQ ID NO: 216 - protein, 20 aa, Artificial Sequence
  Synthetic polypeptide
CFSRYPDHMKQHDFFKSAMP

SEQ ID NO: 217 - protein, 14 aa, Artificial Sequence
  Synthetic polypeptide
DHMKQHDFFKSAMP

SEQ ID NO: 218 - protein, 13 aa, Artificial Sequence
  Synthetic polypeptide
DFFKSAMPEGYVQ

SEQ ID NO: 219 - protein, 20 aa, Artificial Sequence
  Synthetic polypeptide
DFFKSAMPEGYVQERTIFFK

SEQ ID NO: 220 - protein, 12 aa, Artificial Sequence
  Synthetic polypeptide
EGYVQERTIFFK

SEQ ID NO: 221 - protein, 7 aa, Artificial Sequence
  Synthetic polypeptide
ERTIFFK

SEQ ID NO: 222 - protein, 16 aa, Artificial Sequence
  Synthetic polypeptide
ERTIFFKDDGNYKTRA

SEQ ID NO: 223 - protein, 12 aa, Artificial Sequence
  Synthetic polypeptide
DDGNYKTRAEVK

SEQ ID NO: 224 - protein, 15 aa, Artificial Sequence
  Synthetic polypeptide
DDGNYKTRAEVKFEG

SEQ ID NO: 225 - protein, 8 aa, Artificial Sequence
  Synthetic polypeptide
DGNYKTRA

SEQ ID NO: 226 - protein, 6 aa, Artificial Sequence
  Synthetic polypeptide
EVKFEG

SEQ ID NO: 227 - protein, 9 aa, Artificial Sequence
  Synthetic polypeptide
EGDTLVNRI

SEQ ID NO: 228 - protein, 14 aa, Artificial Sequence
  Synthetic polypeptide
EGDTLVNRIELKGI

SEQ ID NO: 229 - protein, 7 aa, Artificial Sequence
  Synthetic polypeptide
DTLVNRI

SEQ ID NO: 230 - protein, 12 aa, Artificial Sequence
  Synthetic polypeptide
DTLVNRIELKGI

SEQ ID NO: 231 - protein, 15 aa, Artificial Sequence
  Synthetic polypeptide
DTLVNRIELKGIDFK

SEQ ID NO: 232 - protein, 16 aa, Artificial Sequence
  Synthetic polypeptide
DTLVNRIELKGIDFKE

SEQ ID NO: 233 - protein, 8 aa, Artificial Sequence
  Synthetic polypeptide
ELKGIDFK

SEQ ID NO: 234 - protein, 10 aa, Artificial Sequence
  Synthetic polypeptide
EDGNILGHKL

SEQ ID NO: 235 - protein, 9 aa, Artificial Sequence
  Synthetic polypeptide
DGNILGHKL

SEQ ID NO: 236 - protein, 22 aa, Artificial Sequence
  Synthetic polypeptide
DGNILGHKLEYNYNSHNVYIMA

SEQ ID NO: 237 - protein, 13 aa, Artificial Sequence
  Synthetic polypeptide
EYNYNSHNVYIMA

SEQ ID NO: 238 - protein, 17 aa, Artificial Sequence
  Synthetic polypeptide
DKQKNGIKVNFKIRHNI

SEQ ID NO: 239 - protein, 18 aa, Artificial Sequence
  Synthetic polypeptide
DKQKNGIKVNFKIRHNIE

SEQ ID NO: 240 - protein, 7 aa, Artificial Sequence
  Synthetic polypeptide
DGSVQLA

SEQ ID NO: 241 - protein, 17 aa, Artificial Sequence
  Synthetic polypeptide
DGSVQLADHYQQNTPIG

SEQ ID NO: 242 - protein, 10 aa, Artificial Sequence
  Synthetic polypeptide
DHYQQNTPIG

SEQ ID NO: 243 - protein, 17 aa, Artificial Sequence
  Synthetic polypeptide
DHYQQNTPIGDGPVLLP

SEQ ID NO: 244 - protein, 7 aa, Artificial Sequence
  Synthetic polypeptide
DGPVLLP

SEQ ID NO: 245 - protein, 20 aa, Artificial Sequence
  Synthetic polypeptide
DGPVLLPDNHYLSTQSALSK

SEQ ID NO: 246 - protein, 23 aa, Artificial Sequence
  Synthetic polypeptide
DGPVLLPDNHYLSTQSALSKDPN

SEQ ID NO: 247 - protein, 13 aa, Artificial Sequence
  Synthetic polypeptide
DNHYLSTQSALSK

SEQ ID NO: 248 - protein, 19 aa, Artificial Sequence
  Synthetic polypeptide
DNHYLSTQSALSKDPNEKR

SEQ ID NO: 249 - protein, 25 aa, Artificial Sequence
  Synthetic polypeptide
DNHYLSTQSALSKDPNEKRDHMVLL

SEQ ID NO: 250 - protein, 12 aa, Artificial Sequence
  Synthetic polypeptide
DPNEKRDHMVLL

SEQ ID NO: 251 - protein, 25 aa, Artificial Sequence
  Synthetic polypeptide
DPNEKRDHMVLLEFVTAAGITLGMD

SEQ ID NO: 252 - protein, 23 aa, Artificial Sequence
  Synthetic polypeptide
DHMVLLEFVTAAGITLGMDELYK

SEQ ID NO: 253 - protein, 23 aa, Artificial Sequence
  Synthetic polypeptide
DHMVLLEFVTAAGITLGMDELYK

SEQ ID NO: 254 - protein, 13 aa, Artificial Sequence
  Synthetic polypeptide
EFVTAAGITLGMD

SEQ ID NO: 255 - protein, 19 aa, Artificial Sequence
  Synthetic polypeptide
EFVTAAGITLGMDELYKLP

SEQ ID NO: 256 - protein, 17 aa, Artificial Sequence
  Synthetic polypeptide
DELYKLPETGGGGGYPY

SEQ ID NO: 257 - protein, 13 aa, Artificial Sequence
  Synthetic polypeptide
ETGGGGGYPYDVP

SEQ ID NO: 258 - protein, 12 aa, Artificial Sequence
  Synthetic polypeptide
DVPDYAQGGQGV

SEQ ID NO: 259 - protein, 26 aa, Artificial Sequence
  Synthetic polypeptide
MVSKGEELFTGVVPILVELDGDVNGH

SEQ ID NO: 260 - protein, 23 aa, Artificial Sequence
  Synthetic polypeptide
GEELFTGVVPILVELDGDVNGHK

SEQ ID NO: 261 - protein, 15 aa, Artificial Sequence
  Synthetic polypeptide
FSVSGEGEGDATYGK

SEQ ID NO: 262 - protein, 12 aa, Artificial Sequence
  Synthetic polypeptide
LPVPWPTLVTTL

SEQ ID NO: 263 - protein, 6 aa, Artificial Sequence
  Synthetic polypeptide
YPDHMK

SEQ ID NO: 264 - protein, 12 aa, Artificial Sequence
  Synthetic polypeptide
YPDHMKQHDFFK

SEQ ID NO: 265 - protein, 6 aa, Artificial Sequence
  Synthetic polypeptide
DDGNYK

SEQ ID NO: 266 - protein, 13 aa, Artificial Sequence
  Synthetic polypeptide
AEVKFEGDTLVNR

SEQ ID NO: 267 - protein, 9 aa, Artificial Sequence
  Synthetic polypeptide
FEGDTLVNR

SEQ ID NO: 268 - protein, 14 aa, Artificial Sequence
  Synthetic polypeptide
GIDFKEDGNILGHK

SEQ ID NO: 269 - protein, 8 aa, Artificial Sequence
  Synthetic polypeptide
LEYNYNSH

SEQ ID NO: 270 - protein, 16 aa, Artificial Sequence
  Synthetic polypeptide
LEYNYNSHNVYIMADK

SEQ ID NO: 271 - protein, 12 aa, Artificial Sequence
  Synthetic polypeptide
YNSHNVYIMADK

SEQ ID NO: 272 - protein, 12 aa, Artificial Sequence
  Synthetic polypeptide
YNSHNVYIMADK

SEQ ID NO: 273 - protein, 14 aa, Artificial Sequence
  Synthetic polypeptide
YNSHNVYIMADKQK

SEQ ID NO: 274 - protein, 10 aa, Artificial Sequence
  Synthetic polypeptide
SHNVYIMADK

SEQ ID NO: 275 - protein, 16 aa, Artificial Sequence
  Synthetic polypeptide
NVYIMADKQKNGIKVN

SEQ ID NO: 276 - protein, 17 aa, Artificial Sequence
  Synthetic polypeptide
VYIMADKQKNGIKVNFK

SEQ ID NO: 277 - protein, 24 aa, Artificial Sequence
  Synthetic polypeptide
TPIGDGPVLLPDNHYLSTQSALSK

SEQ ID NO: 278 - protein, 11 aa, Artificial Sequence
  Synthetic polypeptide
HYLSTQSALSK

SEQ ID NO: 279 - protein, 14 aa, Artificial Sequence
  Synthetic polypeptide
LPETAAGGGGDPAK

SEQ ID NO: 280 - DNA, 45 bp, Artificial Sequence
  Synthetic polynucleotide
ttttttttttctgcagacgtcttacctctaatcgatcgatctccg

SEQ ID NO: 281 - protein, 8 aa, Artificial Sequence
  Synthetic polypeptide
LPETGGGK

SEQ ID NO: 282 - protein, 7 aa, Artificial Sequence
  Synthetic polypeptide
GGGGGHA

SEQ ID NO: 283 - protein, 11 aa, Artificial Sequence
  Synthetic polypeptide
LPETAHHHHHH

SEQ ID NO: 284 - protein, 4 aa, Artificial Sequence
  Synthetic polypeptide
GGGS

SEQ ID NO: 285 - protein, 12 aa, Artificial Sequence
  Synthetic polypeptide
GGGSGGGSGGGS

SEQ ID NO: 286 - protein, 10 aa, Artificial Sequence
  Synthetic polypeptide
GGGSLPETGG

SEQ ID NO: 287 - protein, 11 aa, Artificial Sequence
  Synthetic polypeptide
LPETGHHHHHH

SEQ ID NO: 288 - protein, 6 aa, Artificial Sequence
  Synthetic polypeptide
HHHHHH

SEQ ID NO: 289 - protein, 6 aa, Artificial Sequence
  Synthetic polypeptide
LPXTXT

(19) United States
(12) Patent Application Publication
(10) Pub. No.: US 2014/0030697 A1
(43) Pub. Date: Jan. 30, 2014

(54) SORTASE-MEDIATED MODIFICATION OF VIRAL SURFACE PROTEINS

(71) Applicants: Massachusetts Institutes of Technology, Cambridge, MA (US); Whitehead Institute for Biomedical Research, Cambridge, MA (US)

(72) Inventors: Hidde L. Ploegh, Brookline, MA (US); Gaelen Hess, Somerville, MA (US); Carla Guimaraes, Boston, MA (US); Angela Belcher, Lexington, MA (US)

(73) Assignees: Massachusetts Institute of Technology, Cambridge, MA (US); Whitehead Institute for Biomedical Research, Cambridge, MA (US)

(21) Appl. No.: 13/918,278

(22) Filed: Jun. 14, 2013

Related U.S. Application Data
(60) Provisional application No. 61/659,661, filed on Jun. 14, 2012.

(51) Int. Cl. C12N 7/00

(52) U.S. Cl. CPC C12N 7/00
USPC 435/5; 435/235.1

(57) ABSTRACT

The present invention, in some aspects, provides methods, reagents, and kits for the functionalization of proteins on the surface of viral particles, for example, of bacteriophages, using sortase-mediated transpeptidation reactions. Some aspects of this invention provide methods for the conjugation of an agent, for example, a detectable label, a binding agent, a click-chemistry handle, or a small molecule to a surface protein of a viral particle. Kits comprising reagents useful for the generation of functionalized viral particles are also provided, as are precursor proteins that comprise a sortase recognition motif, and viral particles comprising such precursor proteins. Nucleic acids encoding viral proteins comprising a sortase recognition motif and expression vectors comprising such nucleic acids are also provided.

### RELATED APPLICATIONS

The present application claims priority under 35 U.S.C. §119(e) to U.S. provisional application, U.S. Ser. No. 61/659,661, filed Jun. 14, 2012, the entire contents of which is incorporated herein by reference.

### GOVERNMENT SUPPORT

This invention was made with U.S. government support under grant 5R01AI033456 awarded by the National Institutes of Health and under grant number W911NF-09-0001 awarded by the U.S. Army Research Office. The Government has certain rights in the invention.

### BACKGROUND OF THE INVENTION

Biological surfaces, e.g., surfaces of cells or viruses, can be modified in order to modulate surface function or to confer new functions to such surfaces. Surface functionalization may, for example, include an addition of a detectable label or binding moiety to a surface protein, allowing for detection or isolation of the functionalized cell or virus, or for the generation of new cell-cell or virus-host interactions that do not naturally occur. Functionalization of surface proteins can be achieved by genetic engineering or by chemical modifications. Both approaches are, however, limited in their capabilities, for example, in that many surface proteins do not tolerate insertions above a certain size without suffering impairments in their function or expression, and in that many chemical modifications require non-physiological reaction conditions and are not specific to a single viral surface protein.

### SUMMARY OF THE INVENTION

The present invention stems in part from the recognition that bacterial sortases can be exploited to attach a variety of moieties to proteins on the surface of a virus. Such sortase-mediated modification reactions can be performed under physiological conditions. Methods, reagents, and kits are provided herein that can be used to functionalize proteins on the surface of viral particles via a sortase-mediated transpeptidation reaction. For example, some aspects of the invention provide methods and reagents for the functionalization of a protein on the surface of a virus by the addition of an entity, e.g., a small molecule (e.g., a fluorophore, biotin), a detectable label, a binding agent, a peptide, or a protein (e.g., GFP, an antibody or a fragment thereof, streptavidin). Some of the methods provided herein allow for functionalization of proteins on the surface of a virus in a site-specific manner, and with yields that surpass those of any currently known technologies, including, but not limited to, chemical modification and recombinant technologies (e.g., phage display technology). For example, the methods provided herein are useful for functionalization of phage surface proteins, such as M13 bacteriophage surface proteins.

In one aspect, the present invention provides methods, reagents, and kits for sortase-mediated functionalization of M13 bacteriophage capsid proteins pIII, pVIII, and pIX with various moieties. A comparison to commonly used techniques using chemical modification or genetic engineering demonstrates that the inventive sortase-based technology provided herein yields functionalized viral particles with greater efficiency and greater labeling density than these known methods. Further, some aspects of this disclosure provide a technology that takes advantage of orthogonal sortases that specifically target different recognition sequences, allowing for the functionalization of a plurality of different proteins on the surface of the same viral particle, e.g., with a different modification introduced into each of the different proteins, while maintaining excellent specificity. The methods provided herein are simple and effective for adding a variety of structures on the surface of viruses, and are useful for creating new viral surface modifications that can be exploited for the creation of novel surface interactions.

In some aspects, this invention provides methods of modifying a target protein comprising a sortase recognition motif on the surface of a virus. In some embodiments, the method comprises contacting the target protein with a sortase substrate conjugated to an agent, e.g., a detectable label, a binding agent, a click-chemistry handle, a reactive moiety, or a small molecule, in the presence of a sortase under conditions suitable for the sortase to conjugate the target protein and the sortase substrate. In some embodiments, the target protein comprises an N-terminal sortase recognition motif. In some embodiments, the N-terminal sortase recognition motif comprises an oligoglycine or an oligoalanine sequence. In some embodiments, the oligoglycine and/or the oligoalanine comprises 1-10 N-terminal glycine residues or 1-10 N-terminal alanine residues, respectively. In some embodiments, the sortase substrate comprises a C-terminal sortase recognition motif. In some embodiments, the C-terminal recognition motif is LPXTX, wherein each instance of X independently represents any amino acid residue. In some embodiments, the C-terminal recognition motif is LPETG (SEQ ID NO: 10) or LPETA (SEQ ID NO: 11). In some embodiments, the sortase is sortase A from Staphylococcus aureus (SrtAₐᵤᵣₑᵤₛ) or sortase A from Streptococcus pyogenes (SrtA_{pyogenes}). In some embodiments, the virus is an RNA virus. In some embodiments, the virus is a DNA virus. In some embodiments, the virus is a single-stranded DNA virus. In some embodiments, the virus is a bacteriophage. In some embodiments, the virus is an M13 bacteriophage. In some embodiments, the target protein is a viral capsid protein. In some embodiments, the target protein is an M13 pIII, pVIII, or pIX capsid protein. In some embodiments, the agent is a protein, a carbohydrate, a lipid, a detectable label, a binding agent, a click-chemistry handle, or a small molecule. In some embodiments, the agent is a fluorescent protein, streptavidin, biotin, a fluorophore, an antibody or an antibody fragment, a nucleic acid molecule, an alkyne, an azide, a diene, a dienophile, a thiol, an alkene, an aryne, a tetrazine, a tetrazole, a dithioester, an anthracene, a maleimide, an enone, or an amine. In some embodiments, the method comprises multiple rounds of modifying a target protein on the surface of the same virus, wherein a different target protein is modified in each round. In some embodiments, different target proteins are modified using different sortases which recognize different sortase recognition motifs. For example, in some embodiments, at least one of the target proteins is modified using SrtAₐᵤᵣₑᵤₛ, and at least one other target protein is modified using SrtA_{pyogenes}. In some embodiments, a different agent is conjugated to each different type of target protein, for example, one type of protein, e.g., M13 pIII, may be conjugated to a binding agent, and a different type of protein, e.g., M13 pVIII, may be conjugated to a detectable label. In some embodiments, a virus is provided that comprises a target protein that has been modified by a method described herein.

Some aspects of this invention provide methods of associating viral particles. In some embodiments, the method comprises conjugating a first target protein on the surface of the viral particle with a first binding agent via a sortase-mediated transpeptidation reaction; conjugating a second target protein on the surface of the viral particle with a second binding agent, wherein the second binding agent binds the first binding agent; and incubating a plurality of such viral particles under conditions suitable for the first and the second binding agent of different viral particles to bind each other. In some embodiments, the first binding agent binds the second binding agent directly. In some embodiments, the first binding agent binds the second binding agent indirectly (e.g., via binding to a third binding agent bound by the first binding agent). For example, in some embodiments, the first binding agent may be a first oligonucleotide, the second binding agent may be a second oligonucleotide, and the third binding agent may be a third oligonucleotide that can hybridize simultaneously with the first and the second oligonucleotide. In some embodiments, a method is provided that comprises conjugating a target protein on the surface of a viral particle with a binding agent via a sortase-mediated transpeptidation reaction, wherein the binding agent binds a binding partner on the surface of another viral particle; and incubating a plurality of such viral particles under conditions suitable for the binding agent to bind its binding partner. For example, in some such embodiments, the binding agent is an antibody binding a viral surface antigen. In some embodiments, a method is provided that comprises functionalizing a first population of viral particles with a first binding agent; functionalizing a second population of viral particles with a second binding agent, wherein the first binding agent binds the second binding agent; and incubating a plurality of viral particles from each population together under conditions suitable for the first and the second binding agent of different viral particles to bind each other. In some such embodiments, the viral particles of the first population are different from the viral particles of the second population, e.g., the first population comprises viral particles of elongate shape (e.g., M13) and the second population comprises particles of more spherical shape (e.g., T4 or Qβ). In some embodiments, the viral particles are DNA virus particles. In some embodiments, the viral particles are bacteriophage particles. In some embodiments, the viral particles are M13 bacteriophage particles. In some embodiments, at least one target protein comprises an N-terminal sortase recognition motif. In some embodiments, the N-terminal sortase recognition motif comprises an oligoglycine or an oligoalanine sequence. In some embodiments, the oligoglycine and/or the oligoalanine comprises 1-10 N-terminal glycine residues or 1-10 N-terminal alanine residues, respectively. In some embodiments, at least one of the target proteins comprises a C-terminal sortase recognition motif. In some embodiments, the C-terminal recognition motif is LPXTX, wherein each instance of X independently represents any amino acid residue. In some embodiments, the C-terminal recognition motif is LPETG (SEQ ID NO: 10) or LPETA (SEQ ID NO: 11). In some embodiments, the sortase used for the sortase-mediated transpeptidation of the first target protein is different from the sortase used for the sortase-mediated transpeptidation of the second target protein. In some embodiments, the sortase used for the sortase-mediated transpeptidation of the first target protein is sortase A from Staphylococcus aureus (SrtAₐᵤᵣₑᵤₛ). In some embodiments, the sortase used for the sortase-mediated transpeptidation of the second target protein is sortase A from Streptococcus pyogenes (SrtA_{pyogenes}). In some embodiments, the first and/or the second target protein is a viral capsid protein. In some embodiments, the first and the second target protein is selected from the group consisting of M13 pIII, pVIII, or pIX. In some embodiments, the binding agent is a ligand, a receptor, an extracellular receptor domain, streptavidin, biotin, an antibody, or an antibody fragment. Other suitable binding agents include click chemistry handles, SNAP-, Clip-, ACP-, and MCP-tags, nucleic acid molecules (e.g., complementary DNA strands or non-complementary DNA strands that can hybridize to a third DNA strand), leucine zippers, GFP, as well as toxins, e.g., bacterial and plant toxins.

In some embodiments, viral particles that are functionalized with a binding agent are used in chip-based assays in which the viral particles are conjugated to a solid support. In some embodiments, viral particles that are functionalized with binding agents can be used as a handle in single molecule force spectroscopy, e.g., by linking a bead to a specific target on a surface.

Some aspects of this invention provide viruses comprising a target protein that is conjugated to an agent via a sortase recognition motif. In some embodiments, the target protein is conjugated to the agent via a linker. In some embodiments, the target protein has been conjugated to the agent by a sortase-mediated transpeptidation reaction. In some embodiments, the sortase recognition motif is LPXTX, wherein each instance of X independently represents any amino acid residue. In some embodiments, the sortase recognition motif is LPETG (SEQ ID NO: 10) or LPETA (SEQ ID NO: 11). In some embodiments, the sortase recognition motif is a sequence created by a SrtAₐᵤᵣₑᵤₛ mediated transpeptidation reaction or by a SrtA_{pyogenes} transpeptidation reaction. In some embodiments, the virus is a DNA virus. In some embodiments, the virus is a bacteriophage. In some embodiments, the virus is an M13 bacteriophage. In some embodiments, the target protein is a viral capsid protein. In some embodiments, the target protein is an M13 pIII, pVIII, or pIX capsid protein. In some embodiments, the agent is a protein, a peptide, a detectable label, a binding agent, a click-chemistry handle, or a small molecule. In some embodiments, the agent is a molecule that cannot be genetically encoded, e.g., a carbohydrate, a lipid, or a small molecule. In some embodiments, the agent is a fluorescent protein, streptavidin, biotin, a fluorophore, an antibody, or an antigen-binding antibody fragment. In some embodiments, the virus comprises a plurality of different target proteins conjugated to an agent via a sortase recognition motif. In some embodiments, at least one target protein is modified using SrtAₐᵤᵣₑᵤₛ, and at least one target protein is modified using SrtA_{pyogenes}. In some embodiments, a different agent is conjugated to each different target protein. In some embodiments, the virus is an M13 bacteriophage comprising a pIII capsid protein conjugated to streptavidin via a sortase recognition sequence, and a pVIII capsid protein conjugated to biotin via a sortase recognition sequence.

The present invention, in some aspects, provides viruses comprising a recombinant target protein, wherein the recombinant target protein comprised a sortase recognition motif. In some embodiments, the virus is a DNA virus. In some embodiments, the virus is a bacteriophage. In some embodiments, the virus is an M13 bacteriophage. In some embodiments, the target protein is a capsid protein. In some embodiments, the target protein is an M13 pIII, pVIII, or pIX capsid protein. In some embodiments, the sortase recognition motif is an N-terminal oligoglycine and/or the oligoalanine, comprising 1-10 N-terminal glycine residues or 1-10 N-terminal alanine residues, respectively. In some embodiments, the sortase recognition sequence comprises a C-terminal sortase recognition motif. In some embodiments, the C-terminal recognition motif is LPXTX, wherein each instance of X represents independently any amino acid residue. In some embodiments, the C-terminal recognition motif is LPETG (SEQ ID NO: 10) or LPETA (SEQ ID NO: 11). In some embodiments, the recombinant target protein comprises a loop structure harboring the sortase recognition motif and a protease cleavage site, e.g., a loop structure as disclosed in U.S. patent application Ser. No. 13/642,458, publication number US2013/0122043, by Guimaraes and Ploegh, the entire contents of which are incorporated herein by reference. In some embodiments, the loop structure comprises two cysteine residues that flank the sortase recognition motif and the protease cleavage site. In some embodiments, the loop structure is formed by a disulfide bond between the two cysteine residues. In some embodiments, the loop structure comprises an amino acid sequence derived from a bacterial toxin comprising a loop structure, e.g., an amino acid sequence of at least 40, at least 50, at least 60, at least 70, at least 80, at least 90 amino acid residues that is homologous to, or that is at least 70%, at least 80%, at least 90%, at least 95% or at least 98% identical to the sequence of a bacterial toxin. In some embodiments, the bacterial toxin is a bacterial toxin that comprises a protease-sensitive loop. In some embodiments, the bacterial toxin is a bacterial exotoxin. In some embodiments, the toxin is an AB₅ toxin. In some embodiments, the toxin is a cholera toxin, Shiga toxin (ST), the Shiga-like toxins (e.g., SLT1, SLT2, SLT2c, and SLT2e), E. coli heat labile enterotoxins LT-I (e.g., the two variants LT-Ih from human isolates and LT-Ip from porcine isolates), LT-IIa, and LT-IIB, or pertussis toxin (PT). The sequences of these and other suitable toxins are well known to those of skill in the art. See, e.g., U.S. patent application Ser. No. 13/642,458, publication number US2013/0122043, by Guimaraes and Ploegh, the entire contents of which are incorporated herein by reference. Some aspects of this invention provide engineered viral capsid proteins comprising such artificial loop structures harboring a sortase recognition motif and a protease cleavage site. It will be apparent to those of skill in the art that the methods, reagents, and strategies for engineering target proteins to comprise cleavable loop structures with sortase recognition motifs can be applied to viral capsid proteins, as described in more detail herein, but is not limited to such proteins. As will be apparent to those of skill in the art from the instant disclosure, the inventive methods, reagents, and strategies disclosed herein can be applied to install cleavable loop structures comprising a sortase recognition motif on any protein, including, but not limited to cytoskeletal proteins, extracellular matrix proteins, cell surface proteins, plasma proteins, coagulation factors, cell adhesion proteins, hormones and growth factors, receptors, DNA-binding proteins, transcription factors, antibodies and antibody fragments, chaperone proteins, histones, and enzymes. In some embodiments, the present disclosure provides such engineered proteins, e.g., an antibody or antibody fragment, an enzyme, a transcription factor, etc., comprising a cleavable loop structure with a sortase recognition motif. Methods of using such proteins, e.g., in the context of sortase-mediated functionalization of such proteins, described in more detail herein, are also provided.

Some aspects of this invention provide a kit comprising a recombinant nucleic acid encoding a viral capsid protein comprising a sortase recognition motif. In some embodiments, the recombinant nucleic acid is comprised in an expression vector. In some embodiments, the sortase recognition motif is an N-terminal oligoglycine and/or the oligoalanine, comprising 1-10 N-terminal glycine residues or 1-10 N-terminal alanine residues, respectively. In some embodiments, the sortase recognition motif is a C-terminal LPXTX sequence, wherein each instance of X represents independently any amino acid residue. In some embodiments, the C-terminal recognition motif is LPETG (SEQ ID NO: 10) or LPETA (SEQ ID NO: 11). In some embodiments, the kit further comprises a sortase. In some embodiments, the kit comprises SrtAₐᵤᵣₑᵤₛ and/or SrtA_{pyogenes}. In some embodiments, the kit further comprises a substrate comprising a sortase recognition motif conjugated to an agent. In some embodiments, the sortase catalyzes a transpeptidation reaction involving the sortase recognition motif comprised in the viral capsid protein. In some embodiments, the kit further comprises a buffer or reagent useful for carrying out a sortase-mediated transpeptidation reaction.

The above summary is intended to provide an overview over some aspects of this invention and is not to be construed to limit the invention in any way. Additional aspects, advantages, and embodiments of this invention are described herein, and further embodiments will be apparent to those of skill in the art based on the instant disclosure. The entire contents of all references cited above and herein are hereby incorporated by reference.

### BRIEF DESCRIPTION OF THE DRAWINGS

FIG. 1. M13 bacteriophage structure and sortase schemes. M13 bacteriophage is composed of five capsid proteins. pVIII is the major capsid protein with ˜2700 copies on each phage particle. The pVII and pIX are located at one end and start the assembly process, while pIII and pVI are at the other end and cap the phage. Note: the image is not to scale (a). The mechanism of chemo-enzymatic labeling for sortase A enzymes from Staphylococcus aureus (SrtAₐᵤᵣₑᵤₛ-left) and Streptococcus pyogenes (SrtA_{pyogenes}-right) (SEQ ID NOs: 78, 91, 92 and 126) (b).

FIG. 2. pIII labeling. G₅-pIII (SEQ ID NO: 77) modified phage was incubated with SrtAₐᵤᵣₑᵤₛ and K(biotin)-LPETGG peptide (SEQ ID NO: 13) (a), or GFP-LPETG (SEQ ID NO: 10) (b), for 3 hrs at 37° C. or room temperature, respectively. The reactions were monitored by SDS-PAGE under reducing conditions followed by immunoblotting using streptavidin-HRP (a-top panel) or an anti-pIII antibody (a-bottom panel and b). There are five copies of pIII for each phage and the molecular weight markers are shown on the left. The unidentified anti-pIII reactive protein (*) is attributed to proteolyzed pIII. The identity of the GFP-pIII fusion product was determined by mass spectrometry. The amino acid sequences are as follows:

(SEQ ID NO: 14)
MVSKGEELFT GVVPILVELD GDVNGHKFSV SGEGEGDATY GKLTLKFICT
TGKLPVPWPT LVTTLTYGVQ CFSRYPDHMK QHDFFKSAMP EGYVQERTIF
FKDDGNYKTR AEVKFEGDTL VNRIELKGID FKEDGNILGH KLEYNYNSHN
VYIMADKQKN GIKVNFKIRH NIEDGSVQLA DHYQQNTPIG DGPVLLPDNH
YLSTQSALSK DPNEKRDHMV LLEFVTAAGI TLGMDELYK [symbol] [symbol]
S HTENSFTNVW KDDKTLDRYA NYEGCLWNAT GVVVCTGDET
QCYGTWVPIG LAIPENEGGG SEGGGSEGGG SEGGGTKPPE YGDTPIPGYT
YINPLDGTYP PGTEQNPANP NPSLEESQPL NTFMFQNNRF RNRQGALTVY
TGTVTQGTDP VKTYYQYTPV SSKAMYDAYW NGKFRDCAFH SGFNEDLFVC
EYQGQSSDLP QPPVNAGGGS GGGSGGGSEG GGSEGGGSEG GGSEGGGSGG
GSGSGDFDYE KMANANKGAM TENADENALQ SDAKGKLDSV ATDYGAAIDG
FIGDVSGLAN GNGATGDFAG SNSQMAQVGD GDNSPLMNNF RQYLPSLPQS
VECRPFVFGA GKPYEFSIDC DKINLFRGVF AFLLYVATFM YVFSTFANIL
RNKES.

The sequences of pIII and GFP are shown in underline and double underline, respectively. The peptides identified are in bold. The tryptic peptide comprising the GFP C-terminus, followed by the SrtAaureus cleavage site, fused to the N-terminal glycines of pIII is italicized.

FIG. 3. pIX labeling. G₅HA-pIX (SEQ ID NO: 77) modified phage was incubated with SrtAₐᵤᵣₑᵤₛ and K(biotin)-LPETGG peptide (SEQ ID NO: 13) (a), or GFP-LPETG (SEQ ID NO: 10) (b), at 37° C. and room temperature, respectively, for the times indicated. The reactions were monitored by SDS-PAGE under reducing conditions followed by immunoblotting using streptavidin-HRP (a-top panel) or an anti-HA antibody (a-bottom panel and b). There are five copies of pIX for each phage and the molecular weight markers are shown on the left. The identity of the GFP-pIX fusion product was determined by mass spectrometry. The amino acid sequences are as follows:

(SEQ ID NO: 15)
MVSKGEELFT GVVPILVELD GDVNGHKFSV SGEGEGDATY GKLTLKFICT
TGKLPVPWPT LVTTLTYGVQ CFSRYPDHMK QHDFFKSAMP EGYVQERTIF
FKDDGNYKTR AEVKFEGDTL VNRIELKGID FKEDGNILGH KLEYNYNSHN
VYIMADKQKN GIKVNFKIRH NIEDGSVQLA DHYQQNTPIG DGPVLLPDNH
YLSTQSALSK DPNEKRDHMV LLEFVTAAGI TLGM [symbol] [symbol]
DVPDYAQGG QGVDMSVLVY SFASFVLGWC LRSGITYFTR LMETSS.

The sequences of GFP and pIX are underlined and double underlined, respectively. The peptides identified are in bold. The AspN digestion-resultant peptide comprising the GFP C-terminus, followed by the SrtAₐᵤᵣₑᵤₛ cleavage site, fused to the N-terminal glycines of pIX is italicized.

FIG. 4. pVIII labeling. A₂G₄-pVIII modified phage was incubated with SrtA_{pyogenes} and K(biotin)-LPETAA (SEQ ID NO: 12) peptide (a), or GFP-LPETA (SEQ ID NO: 11) (b), at 37° C. for the times indicated in the figure. The reactions were monitored by SDS-PAGE under reducing conditions followed by immunoblotting using streptavidin-HRP (a) or an anti-GFP antibody (b). There are 2700 copies of pVIII for each phage and the molecular weight markers are shown on the left. The unidentified anti-GFP reactive protein (*) is attributed to proteolyzed GFP forming an intermediate with SrtA_{pyogenes}. The identity of the GFP-pVIII fusion product was determined by mass spectrometry. The amino acid sequences are as follows:

(SEQ ID NO: 16)
MVSKGEELFT GVVPILVELD GDVNGHKESV SGEGEGDATY GKLTLKFICT
TGKLPVPWPT LVTTLTYGVQ CFSRYPDHMK QHDFFKSATP EGYVQQDPTI
FCKDDGNYKT RAEVKFEGDT LVNRIELKGI DFKEDGNILG HKLEYNYNSH
NVYIMADKQK NGTKVNFKTR HNTEDGSVQL ADHYQQNTPI GDGPVLLPDN
HYLSTQSALS KDPNEKRDHM VLLEFVTAAG ITLGMDELYK [symbol] [symbol]
AAFNSL QASATEYIGY AWAMVVVTVG ATTGTKLFKK FTSAS.

The sequences of GFP and pVIII are shown in underline and double underline, respectively. The peptides identified are in bold. The tryptic peptide comprising the GFP C-terminus, followed by the SrtA_{pyogenes} cleavage site, fused to the N-terminal alanines of pVIII is italicized.

FIG. 5. Creation of a multi-phage structure. Schematic representation of the strategy used to build a lampbrush structure (a). Upon labeling of the N-terminus of pIII with streptavidin and of the N-terminus of pVIII with biotin using sortase-mediated reactions, the phage were mixed (SEQ ID NO: 10 and 11). The resulting product was visualized by dynamic light scattering (b) and by atomic force microscopy (c).

FIG. 6. Dual labeling of phage using orthogonal SrtA_{pyogenes} and SrtAₐᵤᵣₑᵤₛ. Schematic representation of the strategy used to couple two different moieties to two different capsid proteins (SEQ ID NOs: 10 and 11) (a). Labeling of pVIII with a K(TAMRA)-LPETAA (SEQ ID NOs: 12) peptide mediated by SrtA_{pyogenes} was followed by labeling of pIII with a single domain antibody directed to Class II MHC as a cell targeting moiety and SrtAₐᵤᵣₑᵤₛ. The final product was analyzed by fluorescent scanning imaging to visualize labeling of pVIII, followed by immunoblotting using an anti-pIII antibody to monitor the efficiency of labeling (b). There are five copies of pIII for each phage. The unidentified anti-pIII reactive proteins (*) are attributed to proteolyzed pIII. Binding of the dual labeled phage to lymphocytic Class II MHC+ cells was observed by flow cytometry (c). The Class II MHC+ enriched cell fraction of the lymph nodes of a C57BL/6 mouse was stained for B220 together with the dual labeled phage (phage-TAMRA-VHH7), TAMRA labeled phage (no cell targeting motif, phage-TAMRA), or anti-Class II MHC directly conjugated to TAMRA (TAMRA-VHH7).

FIG. 7. Characterization of the GFP-pIII conjugate by mass spectrometry. The polypeptide corresponding to GFP-pIII was excised from the SDS-PAGE gel and digested with trypsin. The resulting peptides were analyzed by liquid chromatography MS/MS. Peptides positively identified by sequence are highlighted and bold. Sequences correspond, from top to bottom, to SEQ ID NOs 162-209, respectively.

FIG. 8. Characterization of the GFP-pIX conjugate by mass spectrometry. The polypeptide corresponding to GFP-pIII was excised from the SDS-PAGE gel and digested with AspN. The resulting peptides were analyzed by liquid chromatography MS/MS. Peptides positively identified by sequence are highlighted and bold. Sequences correspond, from top to bottom, to SEQ ID NOs 210-258, respectively.

FIG. 9. Characterization of the GFP-pVIII conjugate by mass spectrometry. The polypeptide corresponding to GFP-pVIII was excised from the SDS-PAGE gel and digested with trypsin. The resulting peptides were analyzed by liquid chromatography MS/MS. Peptides positively identified by sequence are highlighted and bold. Sequences correspond, from top to bottom, to SEQ ID NOs 259-279, respectively.

FIG. 10. pIII labeling with streptavidin G₅-pIII phage (SEQ ID NO: 77) was incubated with SrtAₐᵤᵣₑᵤₛ and streptavidin containing a C-terminal LPETG (SEQ ID NO: 10) motif in each monomer. The reactions were monitored by SDS-PAGE under reducing conditions followed by immunoblotting using an anti-pIII antibody. There are five copies of pIII for each phage and the molecular weight markers are shown on the left. The unidentified anti-pIII reactive protein (*) is attributed to proteolyzed pIII. The identity of the streptavidin-pIII fusion product was determined by mass spectrometry. The amino acid sequences are as follows:

(SEQ ID NO: 17)
MAEAGITGTW YNQLGSTFIV TAGADGALTG TYESAVGNAE SRYVLTGRYD
SAPATDGSGT ALGWTVAWKN NYRNAHSATT WSGQYVGGAE ARINTQWLLT
SGTTEANAWK STLVGHDTFT K [symbol]
SHTENSFTNV WKDDKTLDRY ANYEGCLWNA TGVVVCTGDE TQCYGTWVPI
GLAIPENEGG GSEGGGSEGG GSEGGGTKPP EYGDTPIPGY TYINPLDGTY
PPGTEQNPAN PNPSLEESQP LNTFMFQNNR FRNRQGALTV YTGTVTQGTD
PVKTYYQYTP VSSKAMYDAY WNGKFRDCAF HSGFNEDLFV CEYQGQSSDL
PQPPVNAGGG SGGGSGGGSE GGGSEGGGSE GGGSEGGGSG GGSGSGDFDY
EKMANANKGA MTENADENAL QSDAKGKLDS VATDYGAAID GFIGDVSGLA
NGNGATGDFA GSNSQMAQVG DGDNSPLMNN FRQYLPSLPQ SVECRPFVFG
AGKPYEFSID CDKINLFRGV FAFLLYVATF MYVFSTFANI LRNKES.

The sequences of streptavidin monomer and pIII and are shown in underline and double underline, respectively. The peptides identified are in bold. The tryptic peptide comprising the streptavidin C-terminus, followed by the SrtAₐᵤᵣₑᵤₛ cleavage site, fused to the N-terminal glycines of pIII is italicized.

FIG. 11. AFM characterization of lampbrush phage structure. Phage with the N-terminus of pIII labeled with streptavidin and phage with the N-terminus of pVIII conjugated to biotin were created using sortase-mediated reactions. The phage preparations were visualized by atomic force microscopy (AFM) before (top right and top left panels) and after mixing (bottom panels).

FIG. 12. Labeling of loop-pIII. Schematic for C-terminal labeling using the loop structure (SEQ ID NOs: 10 and 13) (a). LoopXa-pIII phage was incubated with SrtAₐᵤᵣₑᵤₛ, Factor Xa, and GGGK(TAMRA) (SEQ ID NO: 127) (b). The reactions were monitored by SDS-PAGE under reducing and non-reducing conditions followed by fluorescent imaging and immunoblotting with an anti-pIII antibody. The molecular weight markers are shown on the left.

FIG. 13. Orthogonal labeling of phage with three fluorophores. Schematic representation of the strategy used for triple labeling of a single phage particle (SEQ ID NOs: 10 and 11) (a). TriSrt phage (lane 1) was incubated with SrtA_{pyogenes} and K(TAMRA)-LPETAA (SEQ ID NO: 12) and purified by PEG8000/NaCl precipitation (lane 2). The TAMRA-pVIII labeled triSrt phage was incubated with Factor Xa, SrtAₐᵤᵣₑᵤₛ, FAM-LPETGG (SEQ ID NO: 13), and/or G₃-Alexa647, and purified. These reactions were monitored by SDS-PAGE under non-reducing conditions, followed by fluorescent imaging and immunoblotting with an anti-pIII or anti-HA antibody (b). The molecular weight markers are indicated on the left.

FIG. 14. Building phage by DNA hybridization. Scheme of the multi-phage final structure upon DNA hybridization (a). TriSrt Phage was incubated with DNA-peptides, SrtAₐᵤᵣₑᵤₛ and purified by PEG8000/NaCl precipitation. The reactions were monitored by SDS-PAGE under non-reducing conditions, followed by fluorescent imaging (b). The samples with DNA-peptide alone had a concentration of 650 nM instead of 50 μM. The molecular weight markers are shown on the left. Phage were linked and imaged by atomic force microscopy (c). The length of the phage structures were measured and collected in a histogram and analyzed by dynamic light scattering (d). Fluorescently labeled phage were connected and imaged by fluorescent microscopy (e).

FIG. 15. C-terminal display on pIII, pVI, and pIX. DNA sequences encoding LPETGG-(HA) (SEQ ID NO: 13), GGGS-LPETGG-(HA) (SEQ ID NO: 286), and (GGGS)₃-LPETGG-(HA) (SEQ ID NO: 90) were inserted genetically at the C-terminus of pIII, pIX, and pVI. To determine whether the inserts had been incorporated into the genome, the ligation reactions were analyzed by PCR using one of the insertion oligonucleotides from the ligation and a second primer annealing in an unmodified part of the phage vector.

FIG. 16. Labeling of pIII with G₃-CtxB. LoopXa-pIII phage was incubated with SrtAₐᵤᵣₑᵤₛ, Factor Xa, and G₃-CtxB. The reactions were monitored by SDS-PAGE under non-reducing conditions followed by immunoblotting with an anti-pIII antibody and anti-CtxB antibody. The molecular weight markers are shown on the left. The identity of the CtxB-pIII fusion product was determined by mass-spectrometry (see sequence in the Figure). The peptides identified are highlighted in bold in the Figure.

(SEQ ID NO: 18)
EPW [symbol] HNTQIHT LNDKIFSYTE
SLAGKREMAI ITFKNGATFQ VEVPGSQHID SQKKAIERMK DTLRIAYLTE
AKVEKLCVWN NKTPHAIAAI SMAN

[symbol]

YANYEGCLWN ATGVVVCTGD ETQCYGTWVP IGLAIPENEG GGSEGGGSEG
GGSEGGGTKP PEYGDTPIPG YTYINPLDGT YPPGTEQNPA NPNPSLEESQ
PLNTFMFQNN RFRNRQGALT VYTGTVTQGT DPVKTYYQYT PVSSKAMYDA
YWNGKFRDCA FHSGFNEDLF VCEYQGQSSD LPQPPVNAGG GSGGGSGGGS
EGGGSEGGGS EGGGSEGGGS GGGSGSGDFD YEKMANANKG AMTENADENA
LQSDAKGKLD SVATDYGAAI DGFIGDVSGL ANGNGATGDF AGSNSQMAQV
GDGDNSPLMN NFRQYLPSLP QSVECRPFVF GAGKPYEFSI DCDKINLFRG
VFAFLLYVAT FMYVFSTFAN ILRNKES.

The amino acid sequence of pIII is underlined and the sequence of CtxB is shown in bold in the sequence above. The chymotryptic peptide comprising the C-terminus of the loop, followed by the SrtAₐᵤᵣₑᵤₛ cleavage site, fused to the N-terminal glycines of CtxB is double underlined. The cysteine residues forming the S—S bond are framed.

FIG. 17. Building end-to-end phage dimers. Schematic representation of the strategy used to build end-to-end phage dimers (a). G₅-pIII phage (SEQ ID NO: 77), loopXa-pIII phage, Factor Xa, and SrtAₐᵤᵣₑᵤₛ were incubated at room temperature for 60 hrs and purified by PEG8000/NaCl precipitation. The resulting product was visualized by atomic force microscopy (b).

FIG. 18—Conjugation of DNA to peptides. Thiolated DNA was conjugated to either (maleimide)-LPETGG (SEQ ID NO: 13) or GGGK(maleimide) peptide SEQ ID NO: 127. The conjugated peptides were analyzed by MALDI-TOF mass-spectrometry (a) and by TBE-Urea PAGE followed by fluorescent imaging (b).

FIG. 19. Characterization of DNA hybridized phage multimers. TriSrt phage labeled with different DNA oligonucleotides were linked by DNA C and F. The resultant phage particles were imaged by atomic force microscopy (top panel). Only individual phage particles were observed in the absence of DNA C and F (bottom panel).

FIG. 20. Characterization of phage trimers after digest with restriction enzymes. Multi-phage structures were digested with restriction enzymes AatII (top panel), AgeI (middle panel), or both (bottom panel) and analyzed by atomic force microscopy.

FIG. 21. Characterization of phage multimers by fluorescent microscopy. Individual triSrt phage particles fluorescently labeled on their pVIII were labeled with DNA on their ends by sortase and linked together. The multi-phage structures were imaged by fluorescent microscopy only when the crosslinking oligonucleotides were present.

### DEFINITIONS

Definitions of specific functional groups and chemical terms are described in more detail below. For purposes of this invention, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5th Edition, John Wiley & Sons, Inc., New York, 2001; Larock, Comprehensive Organic Transformations, VCH Publishers, Inc., New York, 1989; Carruthers, Some Modern Methods of Organic Synthesis, 3rd Edition, Cambridge University Press, Cambridge, 1987.

The term “aliphatic,” as used herein, includes both saturated and unsaturated, nonaromatic, straight chain (i.e., unbranched), branched, acyclic, and cyclic (i.e., carbocyclic) hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups. In certain embodiments, as used herein, “aliphatic” is used to indicate those aliphatic groups (cyclic, acyclic, substituted, unsubstituted, branched or unbranched) having 1-20 carbon atoms (C₁₋₂₀ aliphatic). In certain embodiments, the aliphatic group has 1-10 carbon atoms (C₁₋₁₀ aliphatic). In certain embodiments, the aliphatic group has 1-6 carbon atoms (C₁₋₆ aliphatic). In certain embodiments, the aliphatic group has 1-5 carbon atoms (C₁₋₅ aliphatic). In certain embodiments, the aliphatic group has 1-4 carbon atoms (C₁₋₄ aliphatic). In certain embodiments, the aliphatic group has 1-3 carbon atoms (C₁₋₃ aliphatic). In certain embodiments, the aliphatic group has 1-2 carbon atoms (C₁₋₂ aliphatic). Aliphatic group substituents include, but are not limited to, any of the substituents described herein, that result in the formation of a stable moiety.

The term “alkyl,” as used herein, refers to saturated, straight- or branched-chain hydrocarbon radicals derived from a hydrocarbon moiety containing between one and twenty carbon atoms by removal of a single hydrogen atom. In some embodiments, the alkyl group employed in the invention contains 1-20 carbon atoms (C₁₋₂₀alkyl). In another embodiment, the alkyl group employed contains 1-15 carbon atoms (C₁₋₁₅alkyl). In another embodiment, the alkyl group employed contains 1-10 carbon atoms (C₁₋₁₀alkyl). In another embodiment, the alkyl group employed contains 1-8 carbon atoms (C₁₋₈alkyl). In another embodiment, the alkyl group employed contains 1-6 carbon atoms (C₁₋₆alkyl). In another embodiment, the alkyl group employed contains 1-5 carbon atoms (C₁₋₅alkyl). In another embodiment, the alkyl group employed contains 1-4 carbon atoms (C₁₋₄alkyl). In another embodiment, the alkyl group employed contains 1-3 carbon atoms (C₁₋₃alkyl). In another embodiment, the alkyl group employed contains 1-2 carbon atoms (C₁₋₂alkyl). Examples of alkyl radicals include, but are not limited to, methyl, ethyl, n-propyl, isopropyl, n-butyl, iso-butyl, sec-butyl, sec-pentyl, iso-pentyl, tert-butyl, n-pentyl, neopentyl, n-hexyl, sec-hexyl, n-heptyl, n-octyl, n-decyl, n-undecyl, dodecyl, and the like, which may bear one or more substituents. Alkyl group substituents include, but are not limited to, any of the substituents described herein, that result in the formation of a stable moiety. The term “alkylene,” as used herein, refers to a biradical derived from an alkyl group, as defined herein, by removal of two hydrogen atoms. Alkylene groups may be cyclic or acyclic, branched or unbranched, substituted or unsubstituted. Alkylene group substituents include, but are not limited to, any of the substituents described herein, that result in the formation of a stable moiety.

The term “alkenyl,” as used herein, denotes a monovalent group derived from a straight- or branched-chain hydrocarbon moiety having at least one carbon-carbon double bond by the removal of a single hydrogen atom. In certain embodiments, the alkenyl group employed in the invention contains 2-20 carbon atoms (C₂₋₂₀alkenyl). In some embodiments, the alkenyl group employed in the invention contains 2-15 carbon atoms (C₂₋₁₅alkenyl). In another embodiment, the alkenyl group employed contains 2-10 carbon atoms (C₂₋₁₀alkenyl). In still other embodiments, the alkenyl group contains 2-8 carbon atoms (C₂₋₈alkenyl). In yet other embodiments, the alkenyl group contains 2-6 carbons (C₂₋₆alkenyl). In yet other embodiments, the alkenyl group contains 2-5 carbons (C₂₋₅alkenyl). In yet other embodiments, the alkenyl group contains 2-4 carbons (C₂₋₄alkenyl). In yet other embodiments, the alkenyl group contains 2-3 carbons (C₂₋₃alkenyl). In yet other embodiments, the alkenyl group contains 2 carbons (C₂alkenyl). Alkenyl groups include, for example, ethenyl, propenyl, butenyl, 1-methyl-2-buten-1-yl, and the like, which may bear one or more substituents. Alkenyl group substituents include, but are not limited to, any of the substituents described herein, that result in the formation of a stable moiety. The term “alkenylene,” as used herein, refers to a biradical derived from an alkenyl group, as defined herein, by removal of two hydrogen atoms. Alkenylene groups may be cyclic or acyclic, branched or unbranched, substituted or unsubstituted. Alkenylene group substituents include, but are not limited to, any of the substituents described herein, that result in the formation of a stable moiety.

The term “alkynyl,” as used herein, refers to a monovalent group derived from a straight- or branched-chain hydrocarbon having at least one carbon-carbon triple bond by the removal of a single hydrogen atom. In certain embodiments, the alkynyl group employed in the invention contains 2-20 carbon atoms (C₂₋₂₀alkynyl). In some embodiments, the alkynyl group employed in the invention contains 2-15 carbon atoms (C₂₋₁₅alkynyl). In another embodiment, the alkynyl group employed contains 2-10 carbon atoms (C₂₋₁₀alkynyl). In still other embodiments, the alkynyl group contains 2-8 carbon atoms (C₂₋₈alkynyl). In still other embodiments, the alkynyl group contains 2-6 carbon atoms (C₂₋₆alkynyl). In still other embodiments, the alkynyl group contains 2-5 carbon atoms (C₂₋₅alkynyl). In still other embodiments, the alkynyl group contains 2-4 carbon atoms (C₂₋₄alkynyl). In still other embodiments, the alkynyl group contains 2-3 carbon atoms (C₂₋₃alkynyl). In still other embodiments, the alkynyl group contains 2 carbon atoms (C₂alkynyl). Representative alkynyl groups include, but are not limited to, ethynyl, 2-propynyl (propargyl), 1-propynyl, and the like, which may bear one or more substituents. Alkynyl group substituents include, but are not limited to, any of the substituents described herein, that result in the formation of a stable moiety. The term “alkynylene,” as used herein, refers to a biradical derived from an alkynylene group, as defined herein, by removal of two hydrogen atoms. Alkynylene groups may be cyclic or acyclic, branched or unbranched, substituted or unsubstituted. Alkynylene group substituents include, but are not limited to, any of the substituents described herein, that result in the formation of a stable moiety.

The term “aptamer” as used herein refers to a nucleic acid ligand or receptor that binds to a target molecule. In some embodiments, an aptamer binds a target molecule with high affinity, e.g., with an K_{D} of less than 10⁻⁶ M, less than 10⁻⁷ M, less than 10⁻⁸ M, less than 10⁻⁹ M, or less than 10⁻¹⁰ M. In some embodiments, an aptamer binds a target molecule with high specificity, e.g., in that it does not bind a ligand other than the target ligand with an affinity of less than 10⁻⁶ M. Typically, an aptamer forms a secondary structure resulting in a three-dimensional complementarity to the target molecule or a substructure thereof.

The term “carbocyclic” or “carbocyclyl” as used herein, refers to an as used herein, refers to a cyclic aliphatic group containing 3-10 carbon ring atoms (C₃₋₁₀-carbocyclic). Carbocyclic group substituents include, but are not limited to, any of the substituents described herein, that result in the formation of a stable moiety.

The term “heteroaliphatic,” as used herein, refers to an aliphatic moiety, as defined herein, which includes both saturated and unsaturated, nonaromatic, straight chain (i.e., unbranched), branched, acyclic, cyclic (i.e., heterocyclic), or polycyclic hydrocarbons, which are optionally substituted with one or more functional groups, and that further contains one or more heteroatoms (e.g., oxygen, sulfur, nitrogen, phosphorus, or silicon atoms) between carbon atoms. In certain embodiments, heteroaliphatic moieties are substituted by independent replacement of one or more of the hydrogen atoms thereon with one or more substituents. As will be appreciated by one of ordinary skill in the art, “heteroaliphatic” is intended herein to include, but is not limited to, heteroalkyl, heteroalkenyl, heteroalkynyl, heterocycloalkyl, heterocycloalkenyl, and heterocycloalkynyl moieties. Thus, the term “heteroaliphatic” includes the terms “heteroalkyl,” “heteroalkenyl,” “heteroalkynyl,” and the like. Furthermore, as used herein, the terms “heteroalkyl,” “heteroalkenyl,” “heteroalkynyl,” and the like encompass both substituted and unsubstituted groups. In certain embodiments, as used herein, “heteroaliphatic” is used to indicate those heteroaliphatic groups (cyclic, acyclic, substituted, unsubstituted, branched or unbranched) having 1-20 carbon atoms and 1-6 heteroatoms (C₁₋₂₀heteroaliphatic). In certain embodiments, the heteroaliphatic group contains 1-10 carbon atoms and 1-4 heteroatoms (C₁₋₁₀heteroaliphatic). In certain embodiments, the heteroaliphatic group contains 1-6 carbon atoms and 1-3 heteroatoms (C₁₋₆heteroaliphatic). In certain embodiments, the heteroaliphatic group contains 1-5 carbon atoms and 1-3 heteroatoms (C₁₋₅heteroaliphatic). In certain embodiments, the heteroaliphatic group contains 1-4 carbon atoms and 1-2 heteroatoms (C₁₋₄heteroaliphatic). In certain embodiments, the heteroaliphatic group contains 1-3 carbon atoms and 1 heteroatom (C₁₋₃heteroaliphatic). In certain embodiments, the heteroaliphatic group contains 1-2 carbon atoms and 1 heteroatom (C₁₋₂heteroaliphatic). Heteroaliphatic group substituents include, but are not limited to, any of the substituents described herein, that result in the formation of a stable moiety.

The term “heteroalkyl,” as used herein, refers to an alkyl moiety, as defined herein, which contain one or more heteroatoms (e.g., oxygen, sulfur, nitrogen, phosphorus, or silicon atoms) in between carbon atoms. In certain embodiments, the heteroalkyl group contains 1-20 carbon atoms and 1-6 heteroatoms (C₁₋₂₀ heteroalkyl). In certain embodiments, the heteroalkyl group contains 1-10 carbon atoms and 1-4 heteroatoms (C₁₋₁₀ heteroalkyl). In certain embodiments, the heteroalkyl group contains 1-6 carbon atoms and 1-3 heteroatoms (C₁₋₆ heteroalkyl). In certain embodiments, the heteroalkyl group contains 1-5 carbon atoms and 1-3 heteroatoms (C₁₋₅ heteroalkyl). In certain embodiments, the heteroalkyl group contains 1-4 carbon atoms and 1-2 heteroatoms (C₁₋₄ heteroalkyl). In certain embodiments, the heteroalkyl group contains 1-3 carbon atoms and 1 heteroatom (C₁₋₃ heteroalkyl). In certain embodiments, the heteroalkyl group contains 1-2 carbon atoms and 1 heteroatom (C₁₋₂ heteroalkyl). The term “heteroalkylene,” as used herein, refers to a biradical derived from an heteroalkyl group, as defined herein, by removal of two hydrogen atoms. Heteroalkylene groups may be cyclic or acyclic, branched or unbranched, substituted or unsubstituted. Heteroalkylene group substituents include, but are not limited to, any of the substituents described herein, that result in the formation of a stable moiety.

The term “heteroalkenyl,” as used herein, refers to an alkenyl moiety, as defined herein, which further contains one or more heteroatoms (e.g., oxygen, sulfur, nitrogen, phosphorus, or silicon atoms) in between carbon atoms. In certain embodiments, the heteroalkenyl group contains 2-20 carbon atoms and 1-6 heteroatoms (C₂₋₂₀ heteroalkenyl). In certain embodiments, the heteroalkenyl group contains 2-10 carbon atoms and 1-4 heteroatoms (C₂₋₁₀ heteroalkenyl). In certain embodiments, the heteroalkenyl group contains 2-6 carbon atoms and 1-3 heteroatoms (C₂₋₆ heteroalkenyl). In certain embodiments, the heteroalkenyl group contains 2-5 carbon atoms and 1-3 heteroatoms (C₂₋₅ heteroalkenyl). In certain embodiments, the heteroalkenyl group contains 2-4 carbon atoms and 1-2 heteroatoms (C₂₋₄ heteroalkenyl). In certain embodiments, the heteroalkenyl group contains 2-3 carbon atoms and 1 heteroatom (C₂₋₃ heteroalkenyl). The term “heteroalkenylene,” as used herein, refers to a biradical derived from an heteroalkenyl group, as defined herein, by removal of two hydrogen atoms. Heteroalkenylene groups may be cyclic or acyclic, branched or unbranched, substituted or unsubstituted.

The term “heteroalkynyl,” as used herein, refers to an alkynyl moiety, as defined herein, which further contains one or more heteroatoms (e.g., oxygen, sulfur, nitrogen, phosphorus, or silicon atoms) in between carbon atoms. In certain embodiments, the heteroalkynyl group contains 2-20 carbon atoms and 1-6 heteroatoms (C₂₋₂₀ heteroalkynyl). In certain embodiments, the heteroalkynyl group contains 2-10 carbon atoms and 1-4 heteroatoms (C₂₋₁₀ heteroalkynyl). In certain embodiments, the heteroalkynyl group contains 2-6 carbon atoms and 1-3 heteroatoms (C₂₋₆ heteroalkynyl). In certain embodiments, the heteroalkynyl group contains 2-5 carbon atoms and 1-3 heteroatoms (C₂₋₅ heteroalkynyl). In certain embodiments, the heteroalkynyl group contains 2-4 carbon atoms and 1-2 heteroatoms (C₂₋₄ heteroalkynyl). In certain embodiments, the heteroalkynyl group contains 2-3 carbon atoms and 1 heteroatom (C₂₋₃ heteroalkynyl). The term “heteroalkynylene,” as used herein, refers to a biradical derived from an heteroalkynyl group, as defined herein, by removal of two hydrogen atoms. Heteroalkynylene groups may be cyclic or acyclic, branched or unbranched, substituted or unsubstituted.

The term “heterocyclic,” “heterocycles,” or “heterocyclyl,” as used herein, refers to a cyclic heteroaliphatic group. A heterocyclic group refers to a non-aromatic, partially unsaturated or fully saturated, 3- to 10-membered ring system, which includes single rings of 3 to 8 atoms in size, and bi- and tri-cyclic ring systems which may include aromatic five- or six-membered aryl or heteroaryl groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocyclic refers to a non-aromatic 5-, 6-, or 7-membered ring or polycyclic group wherein at least one ring atom is a heteroatom selected from O, S, and N (wherein the nitrogen and sulfur heteroatoms may be optionally oxidized), and the remaining ring atoms are carbon, the radical being joined to the rest of the molecule via any of the ring atoms. Heterocycyl groups include, but are not limited to, a bi- or tri-cyclic group, comprising fused five, six, or seven-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Exemplary heterocycles include azacyclopropanyl, azacyclobutanyl, 1,3-diazatidinyl, piperidinyl, piperazinyl, azocanyl, thiaranyl, thietanyl, tetrahydrothiophenyl, dithiolanyl, thiacyclohexanyl, oxiranyl, oxetanyl, tetrahydrofuranyl, tetrahydropuranyl, dioxanyl, oxathiolanyl, morpholinyl, thioxanyl, tetrahydronaphthyl, and the like, which may bear one or more substituents. Substituents include, but are not limited to, any of the substituents described herein, that result in the formation of a stable moiety.

The term “aryl,” as used herein, refers to an aromatic mono- or polycyclic ring system having 3-20 ring atoms, of which all the ring atoms are carbon, and which may be substituted or unsubstituted. In certain embodiments of the present invention, “aryl” refers to a mono, bi, or tricyclic C₄-C₂₀ aromatic ring system having one, two, or three aromatic rings which include, but are not limited to, phenyl, biphenyl, naphthyl, and the like, which may bear one or more substituents. Aryl substituents include, but are not limited to, any of the substituents described herein, that result in the formation of a stable moiety. The term “arylene,” as used herein refers to an aryl biradical derived from an aryl group, as defined herein, by removal of two hydrogen atoms. Arylene groups may be substituted or unsubstituted. Arylene group substituents include, but are not limited to, any of the substituents described herein, that result in the formation of a stable moiety. Additionally, arylene groups may be incorporated as a linker group into an alkylene, alkenylene, alkynylene, heteroalkylene, heteroalkenylene, or heteroalkynylene group, as defined herein.

The term “heteroaryl,” as used herein, refers to an aromatic mono- or polycyclic ring system having 3-20 ring atoms, of which one ring atom is selected from S, O, and N; zero, one, or two ring atoms are additional heteroatoms independently selected from S, O, and N; and the remaining ring atoms are carbon, the radical being joined to the rest of the molecule via any of the ring atoms. Exemplary heteroaryls include, but are not limited to pyrrolyl, pyrazolyl, imidazolyl, pyridinyl, pyrimidinyl, pyrazinyl, pyridazinyl, triazinyl, tetrazinyl, pyyrolizinyl, indolyl, quinolinyl, isoquinolinyl, benzoimidazolyl, indazolyl, quinolinyl, isoquinolinyl, quinolizinyl, cinnolinyl, quinazolynyl, phthalazinyl, naphthridinyl, quinoxalinyl, thiophenyl, thianaphthenyl, furanyl, benzofuranyl, benzothiazolyl, thiazolynyl, isothiazolyl, thiadiazolynyl, oxazolyl, isoxazolyl, oxadiaziolyl, oxadiaziolyl, and the like, which may bear one or more substituents. Heteroaryl substituents include, but are not limited to, any of the substituents described herein, that result in the formation of a stable moiety. The term “heteroarylene,” as used herein, refers to a biradical derived from an heteroaryl group, as defined herein, by removal of two hydrogen atoms. Heteroarylene groups may be substituted or unsubstituted. Additionally, heteroarylene groups may be incorporated as a linker group into an alkylene, alkenylene, alkynylene, heteroalkylene, heteroalkenylene, or heteroalkynylene group, as defined herein. Heteroarylene group substituents include, but are not limited to, any of the substituents described herein, that result in the formation of a stable moiety.

The term “acyl,” as used herein, is a subset of a substituted alkyl group, and refers to a group having the general formula —C(═O)R^{A}, —C(═O)OR^{A}, —C(═O)—O—C(═O)R^{A}, —C(═O)SR^{A}, —C(═O)N(R^{A})₂, —C(═S)R^{A}, —C(═S)N(R^{A})₂, and —C(═S)S(R^{A}), —C(═NR^{A})R^{A}, —C(═NR^{A})OR^{A}, —C(═NR^{A})SR^{A}, and —C(═NR^{A})N(R^{A})₂, wherein R^{A} is hydrogen; halogen; substituted or unsubstituted hydroxyl; substituted or unsubstituted thiol; substituted or unsubstituted amino; acyl; optionally substituted aliphatic; optionally substituted heteroaliphatic; optionally substituted alkyl; optionally substituted alkenyl; optionally substituted alkynyl; optionally substituted aryl, optionally substituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{A} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety.

The term “acylene,” as used herein, is a subset of a substituted alkylene, substituted alkenylene, substituted alkynylene, substituted heteroalkylene, substituted heteroalkenylene, or substituted heteroalkynylene group, and refers to an acyl group having the general formulae: —R⁰—(C═X¹)—R⁰—, —R⁰—X²(C═X¹)—R⁰—, or —R⁰—X²(C═X¹)X³—R⁰—, where X¹, X², and X³ is, independently, oxygen, sulfur, or NR^{r}, wherein R^{r} is hydrogen or optionally substituted aliphatic, and R⁰ is an optionally substituted alkylene, alkenylene, alkynylene, heteroalkylene, heteroalkenylene, or heteroalkynylene group, as defined herein. Exemplary acylene groups wherein R⁰ is alkylene includes —(CH₂)_{T}—O(C═O)—(CH₂)_{T}—; —(CH₂)_{T}—NR^{r}(C═O)—(CH₂)_{T}—; —(CH₂)_{T}—O(C═NR^{r})—(CH₂)_{T}—; —(CH₂)_{T}—NR^{r}(C═NR^{r})—(CH₂)_{T}—; —(CH₂)_{T}—(C═O)—(CH₂)_{T}—; —(CH₂)_{T}—(C═NR^{r})—(CH₂)_{T}—; —(CH₂)_{T}—S(C═S)—(CH₂)_{T}—; —(CH₂)_{T}—NR^{r}(C═S)—(CH₂)_{T}—; —(CH₂)_{T}—S(C═NR^{r})—(CH₂)_{T}—; —(CH₂)_{T}—O(C═S)—(CH₂)_{T}—; —(CH₂)—(C═S)—(CH₂)—; or —(CH₂)_{T}—S(C═O)—(CH₂)_{T}—, and the like, which may bear one or more substituents; and wherein each instance of T is, independently, an integer between 0 to 20. Acylene substituents include, but are not limited to, any of the substituents described herein, that result in the formation of a stable moiety.

The term “amino,” as used herein, refers to a group of the formula (—NH₂). A “substituted amino” refers either to a mono-substituted amine (—NHR^{h}) of a disubstituted amine (—NR^{h}₂), wherein the R^{h} substituent is any substituent as described herein that results in the formation of a stable moiety (e.g., an amino protecting group; aliphatic, alkyl, alkenyl, alkynyl, heteroaliphatic, heterocyclic, aryl, heteroaryl, acyl, amino, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted). In certain embodiments, the R^{h} substituents of the di-substituted amino group (—NR^{h}₂) form a 5- to 6-membered heterocyclic ring.

The term “hydroxy” or “hydroxyl,” as used herein, refers to a group of the formula (—OH). A “substituted hydroxyl” refers to a group of the formula (—ORⁱ), wherein Rⁱ can be any substituent which results in a stable moiety (e.g., a hydroxyl protecting group; aliphatic, alkyl, alkenyl, alkynyl, heteroaliphatic, heterocyclic, aryl, heteroaryl, acyl, nitro, alkylaryl, arylalkyl, and the like, each of which may or may not be further substituted).

The term “thio” or “thiol,” as used herein, refers to a group of the formula (—SH). A “substituted thiol” refers to a group of the formula (—SR^{r}), wherein R^{r} can be any substituent that results in the formation of a stable moiety (e.g., a thiol protecting group; aliphatic, alkyl, alkenyl, alkynyl, heteroaliphatic, heterocyclic, aryl, heteroaryl, acyl, sulfinyl, sulfonyl, cyano, nitro, alkylaryl, arylalkyl, and the like, each of which may or may not be further substituted).

The term “imino,” as used herein, refers to a group of the formula (═NR^{r}), wherein R^{r} corresponds to hydrogen or any substituent as described herein, that results in the formation of a stable moiety (for example, an amino protecting group; aliphatic, alkyl, alkenyl, alkynyl, heteroaliphatic, heterocyclic, aryl, heteroaryl, acyl, amino, hydroxyl, alkylaryl, arylalkyl, and the like, each of which may or may not be further substituted).

The term “azide” or “azido,” as used herein, refers to a group of the formula (—N₃).

The terms “halo” and “halogen,” as used herein, refer to an atom selected from fluorine (fluoro, —F), chlorine (chloro, —Cl), bromine (bromo, —Br), and iodine (iodo, —I).

The term “agent,” as used herein, refers to any molecule, entity, or moiety that can be conjugated to a sortase recognition motif. For example, an agent may be a protein, an amino acid, a peptide, a polynucleotide, a carbohydrate, a detectable label, a binding agent, a tag, a metal atom, a contrast agent, a catalyst, a non-polypeptide polymer, a synthetic polymer, a recognition element, a lipid, a linker, or chemical compound, such as a small molecule. In some embodiments, the agent is a binding agent, for example, a ligand or a ligand-binding molecule, streptavidin, biotin, an antibody or an antibody fragment. In some embodiments, the agent cannot be genetically encoded. In some such embodiments, the agent is a lipid, a carbohydrate, or a small molecule. Additional agents suitable for use in embodiments of the present invention will be apparent to the skilled artisan. The invention is not limited in this respect.

The term “amino acid,” as used herein, includes any naturally occurring and non-naturally occurring amino acid. There are many known non-natural amino acids any of which may be included in the polypeptides or proteins described herein. See, for example, S. Hunt, The Non-Protein Amino Acids: In Chemistry and Biochemistry of the Amino Acids, edited by G. C. Barrett, Chapman and Hall, 1985. Some non-limiting examples of non-natural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; —CH₃), disubstituted phenylalanines, substituted tyrosines (e.g., further substituted with —C(═O)C₆H₅; —CF₃; —CN; -halo; —NO₂; —CH₃), and statine. In the context of amino acid sequences, “X” or “Xaa” represents any amino acid residue, e.g., any naturally occurring and/or any non-naturally occurring amino acid residue.

The term “antibody”, as used herein, refers to a protein belonging to the immunoglobulin superfamily. The terms antibody and immunoglobulin are used interchangeably. With some exceptions, mammalian antibodies are typically made of basic structural units each with two large heavy chains and two small light chains. There are several different types of antibody heavy chains, and several different kinds of antibodies, which are grouped into different isotypes based on which heavy chain they possess. Five different antibody isotypes are known in mammals, IgG, IgA, IgE, IgD, and IgM, which perform different roles, and help direct the appropriate immune response for each different type of foreign object they encounter. In some embodiments, an antibody is an IgG antibody, e.g., an antibody of the IgG1, 2, 3, or 4 human subclass. Antibodies from mammalian species (e.g., human, mouse, rat, goat, pig, horse, cattle, camel) are within the scope of the term, as are antibodies from non-mammalian species (e.g., from birds, reptiles, amphibia) are also within the scope of the term, e.g., IgY antibodies.

Only part of an antibody is involved in the binding of the antigen, and antigen-binding antibody fragments, their preparation and use, are well known to those of skill in the art. As is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). Suitable antibodies and antibody fragments for use in the context of some embodiments of the present invention include, for example, human antibodies, humanized antibodies, domain antibodies, F(ab′), F(ab′)₂, Fab, Fv, Fc, and Fd fragments, antibodies in which the Fc and/or FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; antibodies in which the FR and/or CDR1 and/or CDR2 and/or light chain CDR3 regions have been replaced by homologous human or non-human sequences; and antibodies in which the FR and/or CDR1 and/or CDR2 regions have been replaced by homologous human or non-human sequences. In some embodiments, so-called single chain antibodies (e.g., ScFv), (single) domain antibodies, and other intracellular antibodies may be used in the context of the present invention. Domain antibodies, camelid and camelized antibodies and fragments thereof, for example, VHH domains, or nanobodies, such as those described in patents and published patent applications of Ablynx NV and Domantis are also encompassed in the term antibody. Further, chimeric antibodies, e.g., antibodies comprising two antigen-binding domains that bind to different antigens, are also suitable for use in the context of some embodiments of the present invention.

The term “antigen-binding antibody fragment,” as used herein, refers to a fragment of an antibody that comprises the paratope, or a fragment of the antibody that binds to the antigen the antibody binds to, with similar specificity and affinity as the intact antibody. Antibodies, e.g., fully human monoclonal antibodies, may be identified using phage display (or other display methods such as yeast display, ribosome display, bacterial display). Display libraries, e.g., phage display libraries, are available (and/or can be generated by one of ordinary skill in the art) that can be screened to identify an antibody that binds to an antigen of interest, e.g., using panning. See, e.g., Sidhu, S. (ed.) Phage Display in Biotechnology and Drug Discovery (Drug Discovery Series; CRC Press; 1^{st} ed., 2005; Aitken, R. (ed.) Antibody Phage Display: Methods and Protocols (Methods in Molecular Biology) Humana Press; 2nd ed., 2009.

The term “binding agent,” as used herein refers to any molecule that binds another molecule with high affinity. In some embodiments, a binding agent binds its binding partner with high specificity. Examples for binding agents include, without limitation, antibodies, antibody fragments, nucleic acid molecules, receptors, ligands, aptamers, and adnectins.

The term “click chemistry” refers to a chemical philosophy introduced by K. Barry Sharpless of The Scripps Research Institute, describing chemistry tailored to generate covalent bonds quickly and reliably by joining small units comprising reactive groups together (see H. C. Kolb, M. G. Finn and K. B. Sharpless (2001). Click Chemistry: Diverse Chemical Function from a Few Good Reactions. Angewandte Chemie International Edition 40 (11): 2004-2021. Click chemistry does not refer to a specific reaction, but to a concept including, but not limited to, reactions that mimic reactions found in nature. In some embodiments, click chemistry reactions are modular, wide in scope, give high chemical yields, generate inoffensive byproducts, are stereospecific, exhibit a large thermodynamic driving force>84 kJ/mol to favor a reaction with a single reaction product, and/or can be carried out under physiological conditions. In some embodiments, a click chemistry reaction exhibits high atom economy, can be carried out under simple reaction conditions, use readily available starting materials and reagents, uses no toxic solvents or use a solvent that is benign or easily removed (preferably water), and/or provides simple product isolation by non-chromatographic methods (crystallisation or distillation).

The term “click chemistry handle,” as used herein, refers to a reactant, or a reactive group, that can partake in a click chemistry reaction. For example, a strained alkyne, e.g., a cyclooctyne, is a click chemistry handle, since it can partake in a strain-promoted cycloaddition (see, e.g., Table 1). In general, click chemistry reactions require at least two molecules comprising click chemistry handles that can react with each other. Such click chemistry handle pairs that are reactive with each other are sometimes referred to herein as partner click chemistry handles. For example, an azide is a partner click chemistry handle to a cyclooctyne or any other alkyne. Exemplary click chemistry handles suitable for use according to some aspects of this invention are described herein, for example, in Tables 1 and 2. Other suitable click chemistry handles are known to those of skill in the art. For two molecules to be conjugated via click chemistry, the click chemistry handles of the molecules have to be reactive with each other, for example, in that the reactive moiety of one of the click chemistry handles can react with the reactive moiety of the second click chemistry handle to form a covalent bond. Such reactive pairs of click chemistry handles are well known to those of skill in the art and include, but are not limited to, those described in Table 1:

TABLE 1
Exemplary click chemistry handles and reactions.

| | |
|---|---|
| 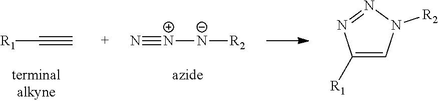 | 1,3-dipolar cycloaddition |
| 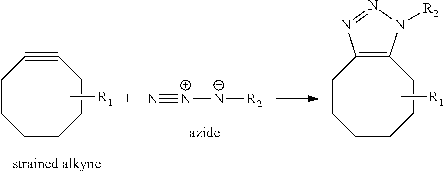 | Strain-promoted cycloaddition |
| 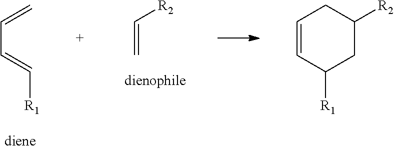 | Diels-Aider reaction |
| 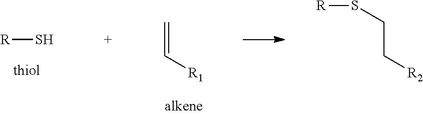 | Thiol-ene reaction |

R, R₁, and R₂ may represent any molecule comprising a sortase recognition motif.
In some embodiments, each ocurrence of R, R₁, and R₂ is independently R_{R}—LPXT—[X]_{y}—, or —[X]_{y}—LPXT—R_{R},
wherein each occurrence of X independently represents any amino acid residue, each occurrence of y is an integer
between 0 and 10, inclusive, and each occurrence of R_{R} independently represents a protein or an agent
(e.g., a protein, peptide, a detectable label, a binding agent, a small molecule, etc.), and, optionally, a linker.

In some embodiments, click chemistry handles are used that can react to form covalent bonds in the absence of a metal catalyst. Such click chemistry handles are well known to those of skill in the art and include the click chemistry handles described in Becer, Hoogenboom, and Schubert, Click Chemistry beyond Metal-Catalyzed Cycloaddition, Angewandte Chemie International Edition (2009) 48: 4900-4908:

TABLE 2
Exemplary click chemistry handles and reactions.

| | Reagent A | Reagent B | Mechanism | Notes on reaction^{[a]} | Reference |
|---|---|---|---|---|---|
| 0 | azide | alkyne | Cu-catalyzed [3 + 2] azide-alkyne | 2 h at 60° C. in H₂O | [9] |
| | | | cycloaddition (CuAAC) | | |
| 1 | azide | cyclooctyne | strain-promoted [3 + 2] azide-alkyne | 1 h at RT | [6-8, 10, 11] |
| | | | cycloaddition (SPAAC) | | |
| 2 | azide | activated | [3 + 2] Huisgen cycloaddition | 4 h at 50° C. | [12] |
| | | alkyne | | | |
| 3 | azide | electron-deficient | [3 + 2] cycloaddition | 12 h at RT in H₂O | [13] |
| | | alkyne | | | |
| 4 | azide | aryne | [3 + 2] cycloaddition | 4 h at RT in THF with crown ether or | [14, 15] |
| | | | | 24 h at RT in CH₃CN | |
| 5 | tetrazine | alkene | Diels-Alder retro-[4 + 2] cycloaddition | 40 min at 25° C. (100% yield) | [36-38] |
| | | | | N₂ is the only by-product | |
| 6 | tetrazole | alkene | 1,3-dipolar cycloaddition | few min UV irradiation and then overnight | [39, 40] |
| | | | (photoclick) | at 4° C. | |
| 7 | dithioester | diene | hetero-Diels-Alder cycloaddition | 10 min at RT | [43] |
| 8 | anthracene | maleimide | [4 + 2] Diels-Alder reaction | 2 days at reflux in toluene | [41] |
| 9 | thiol | alkene | radical addition | 30 min UV (quantitative conv.) or | [19-23] |
| | | | (thio click) | 24 h UV irradiation (>96%) | |
| 10 | thiol | enone | Michael addition | 24 h at RT in CH₃CN | [27] |
| 11 | thiol | maleimide | Michael addition | 1 h at 40° C. in THF or | [24-26] |
| | | | | 16 h at RT in dioxane | |
| 12 | thiol | para-fluoro | nucleophilic substitution | overnight at RT in DMF or | [32] |
| | | | | 60 min at 40° C. in DMF | |
| 13 | amine | para-fluoro | nucleophilic substitution | 20 min MW at 95° C. in NMP as solvent | [30] |

^{[a]}RT = room temperature, DMF = N,N-dimethylformamide, NMP = N-methylpyrolidone, THF = tetrahydrofuran, CH₃CN = acetonitrile.

The term “conjugated” or “conjugation” refers to an association of two molecules, for example, two proteins or a protein and an agent, e.g., a small molecule, with one another in a way that they are linked by a direct or indirect covalent or non-covalent interaction. In certain embodiments, the association is covalent, and the entities are said to be “conjugated” to one another. In some embodiments, a protein is post-translationally conjugated to another molecule, for example, a second protein, a small molecule, a detectable label, a click chemistry handle, or a binding agent, by forming a covalent bond between the protein and the other molecule after the protein has been formed, and, in some embodiments, after the protein has been isolated. In some embodiments, two molecules are conjugated via a linker connecting both molecules. For example, in some embodiments where two proteins are conjugated to each other to form a protein fusion, the two proteins may be conjugated via a polypeptide linker, e.g., an amino acid sequence connecting the C-terminus of one protein to the N-terminus of the other protein. In some embodiments, two proteins are conjugated at their respective C-termini, generating a C—C conjugated chimeric protein. In some embodiments, two proteins are conjugated at their respective N-termini, generating an N—N conjugated chimeric protein. In some embodiments, conjugation of a protein to a peptide is achieved by transpeptidation using a sortase. See, e.g., Ploegh et al., International PCT Patent Application, PCT/US2010/000274, filed Feb. 1, 2010, published as WO/2010/087994 on Aug. 5, 2010, and Ploegh et al., International Patent Application PCT/US2011/033303, filed Apr. 20, 2011, published as WO/2011/133704 on Oct. 27, 2011, the entire contents of each of which are incorporated herein by reference, for exemplary sortases, proteins, recognition motifs, reagents, and methods for sortase-mediated transpeptidation.

The term “detectable label” refers to a moiety that has at least one element, isotope, or functional group incorporated into the moiety which enables detection of the molecule, e.g., a protein or peptide, or other entity, to which the label is attached. Labels can be directly attached (i.e., via a bond) or can be attached by a linker (such as, for example, an optionally substituted alkylene; an optionally substituted alkenylene; an optionally substituted alkynylene; an optionally substituted heteroalkylene; an optionally substituted heteroalkenylene; an optionally substituted heteroalkynylene; an optionally substituted arylene; an optionally substituted heteroarylene; or an optionally substituted acylene, or any combination thereof, which can make up a linker). It will be appreciated that the label may be attached to or incorporated into a molecule, for example, a protein, polypeptide, or other entity, at any position. In general, a detectable label can fall into any one (or more) of five classes: a) a label which contains isotopic moieties, which may be radioactive or heavy isotopes, including, but not limited to, ²H, ³H, ¹³C, ¹⁴C, ¹⁵N, ¹⁸F, ³¹P, ³²P, ³⁵S, ⁶⁷Ga, ^{99m}Tc (Tc-99m), ¹¹¹In, ¹²³I, ¹²⁵I, ¹³¹I, ¹⁵³Gd, ¹⁶⁹Yb, and ¹⁸⁶Re; b) a label which contains an immune moiety, which may be antibodies or antigens, which may be bound to enzymes (e.g., such as horseradish peroxidase); c) a label which is a colored, luminescent, phosphorescent, or fluorescent moieties (e.g., such as the fluorescent label fluorescein-isothiocyanate (FITC); d) a label which has one or more photo affinity moieties; and e) a label which is a ligand for one or more known binding partners (e.g., biotin-streptavidin, FK506-FKBP). In certain embodiments, a label comprises a radioactive isotope, preferably an isotope which emits detectable particles, such as β particles. In certain embodiments, the label comprises a fluorescent moiety. In certain embodiments, the label is the fluorescent label fluorescein-isothiocyanate (FITC). In certain embodiments, the label comprises a ligand moiety with one or more known binding partners. In certain embodiments, the label comprises biotin. In some embodiments, a label is a fluorescent polypeptide (e.g., GFP or a derivative thereof such as enhanced GFP (EGFP)) or a luciferase (e.g., a firefly, Renilla, or Gaussia luciferase). It will be appreciated that, in certain embodiments, a label may react with a suitable substrate (e.g., a luciferin) to generate a detectable signal. Non-limiting examples of fluorescent proteins include GFP and derivatives thereof, proteins comprising fluorophores that emit light of different colors such as red, yellow, and cyan fluorescent proteins. Exemplary fluorescent proteins include, e.g., Sirius, Azurite, EBFP2, TagBFP, mTurquoise, ECFP, Cerulean, TagCFP, mTFP1, mUkG1, mAG1, AcGFP1, TagGFP2, EGFP, mWasabi, EmGFP, TagYPF, EYFP, Topaz, SYFP2, Venus, Citrine, mKO, mKO2, mOrange, mOrange2, TagRFP, TagRFP-T, mStrawberry, mRuby, mCherry, mRaspberry, mKate2, mPlum, mNeptune, T-Sapphire, mAmetrine, mKeima. See, e.g., Chalfie, M. and Kain, S R (eds.) Green fluorescent protein: properties, applications, and protocols Methods of biochemical analysis, v. 47 Wiley-Interscience, Hoboken, N.J., 2006; and Chudakov, D M, et al., Physiol Rev. 90(3):1103-63, 2010, for discussion of GFP and numerous other fluorescent or luminescent proteins. In some embodiments, a label comprises a dark quencher, e.g., a substance that absorbs excitation energy from a fluorophore and dissipates the energy as heat.

The term “linker,” as used herein, refers to a chemical group or molecule covalently linked to a molecule, for example, a protein, and a chemical group or moiety, for example, a click chemistry handle. In some embodiments, the linker is positioned between, or flanked by, two groups, molecules, or moieties and connected to each one via a covalent bond, thus connecting the two. In some embodiments, the linker is an amino acid or a plurality of amino acids. In some embodiments, the linker comprises 1, 2, 3, 4, 5, 6, 7, 8, 9, 10, 11, 12, 13, 14, 15, 16, 17, 18, 19, 20, or more than 20 amino acids. In some embodiments, the linker comprises a poly-glycine sequence. In some embodiments, the linker comprises a GGGGS sequence (SEQ ID NO: 19), or a plurality of such sequences, e.g., a GGGGSGGGGS sequence (SEQ ID NO: 20). In some embodiments, the linker comprises a non-protein structure. In some embodiments, the linker is an organic molecule, group, polymer, or chemical moiety.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems, chemically synthesized, and, optionally, purified. Where appropriate, e.g., in the case of chemically synthesized molecules, nucleic acids can comprise nucleoside analogs such as analogs having chemically modified bases or sugars, and backbone modifications. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof.

The term “small molecule” is used herein to refer to molecules, whether naturally-occurring or artificially created (e.g., via chemical synthesis) that have a relatively low molecular weight. Typically, a small molecule is an organic compound (i.e., it contains carbon). A small molecule may contain multiple carbon-carbon bonds, stereocenters, and other functional groups (e.g., amines, hydroxyl, carbonyls, heterocyclic rings, etc.). In some embodiments, small molecules are monomeric and have a molecular weight of less than about 1500 g/mol. In certain embodiments, the molecular weight of the small molecule is less than about 1000 g/mol or less than about 500 g/mol. In certain embodiments, the small molecule is a drug, for example, a drug that has already been deemed safe and effective for use in humans or animals by the appropriate governmental agency or regulatory body.

The term “sortase,” as used herein, refers to an enzyme able to carry out a transpeptidation reaction conjugating the C-terminus of a protein to the N-terminus of a protein via transamidation. Sortases are also referred to as transamidases, and typically exhibit both a protease and a transpeptidation activity. Various sortases from prokaryotic organisms have been identified. For example, some sortases from Gram-positive bacteria cleave and translocate proteins to proteoglycan moieties in intact cell walls. Among the sortases that have been isolated from Staphylococcus aureus, are sortase A (Srt A) and sortase B (Srt B). Thus, in certain embodiments, a transamidase used in accordance with the present invention is sortase A, e.g., from S. aureus, also referred to herein as SrtAₐᵤᵣₑᵤₛ. In certain embodiments, a transamidase is a sortase B, e.g., from S. aureus, also referred to herein as SrtBₐᵤᵣₑᵤₛ.

Sortases have been classified into 4 classes, designated A, B, C, and D, designated sortase A, sortase B, sortase C, and sortase D, respectively, based on sequence alignment and phylogenetic analysis of 61 sortases from Gram-positive bacterial genomes (Dramsi S, Trieu-Cuot P, Bierne H, Sorting sortases: a nomenclature proposal for the various sortases of Gram-positive bacteria. Res Microbiol. 156(3):289-97, 2005; the entire contents of which are incorporated herein by reference). These classes correspond to the following subfamilies, into which sortases have also been classified by Comfort and Clubb (Comfort D, Clubb R T. A comparative genome analysis identifies distinct sorting pathways in gram-positive bacteria. Infect Immun., 72(5):2710-22, 2004; the entire contents of which are incorporated herein by reference): Class A (Subfamily 1), Class B (Subfamily 2), Class C (Subfamily 3), Class D (Subfamilies 4 and 5). The aforementioned references disclose numerous sortases and recognition motifs. See also Pallen, M. J.; Lam, A. C.; Antonio, M.; Dunbar, K. TRENDS in Microbiology, 2001, 9(3), 97-101; the entire contents of which are incorporated herein by reference. Those skilled in the art will readily be able to assign a sortase to the correct class based on its sequence and/or other characteristics such as those described in Drami, et al., supra. The term “sortase A” is used herein to refer to a class A sortase, usually named SrtA in any particular bacterial species, e.g., SrtA from S. aureus. Likewise “sortase B” is used herein to refer to a class B sortase, usually named SrtB in any particular bacterial species, e.g., SrtB from S. aureus. The invention encompasses embodiments relating to a sortase A from any bacterial species or strain. The invention encompasses embodiments relating to a sortase B from any bacterial species or strain. The invention encompasses embodiments relating to a class C sortase from any bacterial species or strain. The invention encompasses embodiments relating to a class D sortase from any bacterial species or strain.

Amino acid sequences of Srt A and Srt B and the nucleotide sequences that encode them are known to those of skill in the art and are disclosed in a number of references cited herein, the entire contents of all of which are incorporated herein by reference. The amino acid sequences of S. aureus SrtA and SrtB are homologous, sharing, for example, 22% sequence identity and 37% sequence similarity. The amino acid sequence of a sortase-transamidase from Staphylococcus aureus also has substantial homology with sequences of enzymes from other Gram-positive bacteria, and such transamidases can be utilized in the ligation processes described herein. For example, for SrtA there is about a 31% sequence identity (and about 44% sequence similarity) with best alignment over the entire sequenced region of the S. pyogenes open reading frame. There is about a 28% sequence identity with best alignment over the entire sequenced region of the A. naeslundii open reading frame. It will be appreciated that different bacterial strains may exhibit differences in sequence of a particular polypeptide, and the sequences herein are exemplary.

In certain embodiments a transamidase bearing 18% or more sequence identity, 20% or more sequence identity, or 30% or more sequence identity with an S. pyogenes, A. naeslundii, S. mutans, E. faecalis or B. subtilis open reading frame encoding a sortase can be screened, and enzymes having transamidase activity comparable to Srt A or Srt B from S. aureas can be utilized (e.g., comparable activity sometimes is 10% of Srt A or Srt B activity or more).

Thus in some embodiments of the invention the sortase is a sortase A (SrtA). SrtA recognizes the motif LPXTX (wherein each occurrence of X represents independently any amino acid residue), with common recognition motifs being, e.g., LPKTG (SEQ ID NO: 21), LPATG (SEQ ID NO: 22), LPNTG (SEQ ID NO: 23). In some embodiments LPETG (SEQ ID NO: 10) is used as the sortase recognition motif. However, motifs falling outside this consensus may also be recognized. For example, in some embodiments the motif comprises an ‘A’ rather than a ‘T’ at position 4, e.g., LPXAG (SEQ ID NO: 24), e.g., LPNAG (SEQ ID NO: 25). In some embodiments the motif comprises an ‘A’ rather than a ‘G’ at position 5, e.g., LPXTA (SEQ ID NO: 26), e.g., LPNTA (SEQ ID NO: 27). In some embodiments the motif comprises a ‘G’ rather than ‘P’ at position 2, e.g., LGXTG (SEQ ID NO: 28), e.g., LGATG (SEQ ID NO: 29). In some embodiments the motif comprises an ‘I’ rather than ‘L’ at position 1, e.g., IPXTG (SEQ ID NO: 30), e.g., IPNTG (SEQ ID NO: 31) or IPETG (SEQ ID NO: 32). Additional suitable sortase recognition motifs will be apparent to those of skill in the art, and the invention is not limited in this respect. It will be appreciated that the terms “recognition motif” and “recognition sequence”, with respect to sequences recognized by a transamidase or sortase, are used interchangeably.

In some embodiments of the invention the sortase is a sortase B (SrtB), e.g., a sortase B of S. aureus, B. anthracis, or L. monocytogenes. Motifs recognized by sortases of the B class (SrtB) often fall within the consensus sequences NPXTX, e.g., NP[Q/K]-[T/sHN/G/s], such as NPQTN (SEQ ID NO: 33) or NPKTG (SEQ ID NO: 34). For example, sortase B of S. aureus or B. anthracis cleaves the NPQTN (SEQ ID NO: 35) or NPKTG (SEQ ID NO: 36) motif of IsdC in the respective bacteria (see, e.g., Marraffini, L. and Schneewind, O., Journal of Bacteriology, 189(17), p. 6425-6436, 2007). Other recognition motifs found in putative substrates of class B sortases are NSKTA (SEQ ID NO: 37), NPQTG (SEQ ID NO: 38), NAKTN (SEQ ID NO: 39), and NPQSS (SEQ ID NO: 40). For example, SrtB from L. monocytogenes recognizes certain motifs lacking P at position 2 and/or lacking Q or K at position 3, such as NAKTN (SEQ ID NO: 41) and NPQSS (SEQ ID NO: 42) (Mariscotti J F, García-Del Portillo F, Pucciarelli M G. The listeria monocytogenes sortase-B recognizes varied amino acids at position two of the sorting motif. J Biol Chem. 2009 Jan. 7.)

In some embodiments, the sortase is a sortase C (Srt C). Sortase C may utilize LPXTX as a recognition motif, with each occurrence of X independently representing any amino acid residue.

In some embodiments, the sortase is a sortase D (Srt D). Sortases in this class are predicted to recognize motifs with a consensus sequence NA-[E/A/S/H]-TG (Comfort D, supra). Sortase D has been found, e.g., in Streptomyces spp., Corynebacterium spp., Tropheryma whipplei, Thermobifida fusca, and Bifidobacterium longhum. LPXTA (SEQ ID NO: 43) or LAXTG (SEQ ID NO: 44) may serve as a recognition sequence for sortase D, e.g., of subfamilies 4 and 5, respectively subfamily-4 and subfamily-5 enzymes process the motifs LPXTA (SEQ ID NO: 45) and LAXTG (SEQ ID NO: 46), respectively). For example, B. anthracis Sortase C has been shown to specifically cleave the LPNTA (SEQ ID NO: 47) motif in B. anthracis BasI and BasH (see Marrafini, supra).

See Barnett and Scott for description of a sortase that recognizes QVPTGV (SEQ ID NO: 48) motif (Barnett, T C and Scott, J R, Differential Recognition of Surface Proteins in Streptococcus pyogenes by Two Sortase Gene Homologs. Journal of Bacteriology, Vol. 184, No. 8, p. 2181-2191, 2002; the entire contents of which are incorporated herein by reference). Additional sortases, including, but not limited to, sortases recognizing additional sortase recognition motifs are also suitable for use in some embodiments of this invention. For example, sortases described in Chen I, Dorr B M, and Liu D R., A general strategy for the evolution of bond-forming enzymes using yeast display. Proc Natl Acad Sci USA. 2011 Jul. 12; 108(28):11399, the entire contents of which are incorporated herein.

The use of sortases found in any gram-positive organism, such as those mentioned herein and/or in the references (including databases) cited herein is contemplated in the context of some embodiments of this invention. Also contemplated is the use of sortases found in gram negative bacteria, e.g., Colwellia psychrerythraea, Microbulbifer degradans, Bradyrhizobium japonicum, Shewanella oneidensis, and Shewanella putrefaciens. Such sortases recognize sequence motifs outside the LPXTX consensus, for example, LP[Q/K]T[A/S]T (SEQ ID NO: 289). In keeping with the variation tolerated at position 3 in sortases from gram-positive organisms, a sequence motif LPXT[A/S], e.g., LPXTA (SEQ ID NO: 49) or LPSTS (SEQ ID NO: 50) may be used.

Those of skill in the art will appreciate that any sortase recognition motif known in the art can be used in some embodiments of this invention, and that the invention is not limited in this respect. For example, in some embodiments the sortase recognition motif is selected from: LPKTG (SEQ ID NO: 51), LPITG (SEQ ID NO: 52), LPDTA (SEQ ID NO: 53), SPKTG (SEQ ID NO: 54), LAETG (SEQ ID NO: 55), LAATG (SEQ ID NO: 56), LAHTG (SEQ ID NO: 57), LASTG (SEQ ID NO: 58), LAETG (SEQ ID NO: 59), LPLTG (SEQ ID NO: 60), LSRTG (SEQ ID NO: 61), LPETG (SEQ ID NO: 10), VPDTG (SEQ ID NO: 62), IPQTG (SEQ ID NO: 63), YPRRG (SEQ ID NO: 64), LPMTG (SEQ ID NO: 65), LPLTG (SEQ ID NO: 66), LAFTG (SEQ ID NO: 67), LPQTS (SEQ ID NO: 68), it being understood that in various embodiments of the invention the 5^{th} residue may be replaced with any other amino acid residue. For example, the sequence used may be LPXT, LAXT, LPXA, LGXT, IPXT, NPXT, NPQS (SEQ ID NO: 69), LPST (SEQ ID NO: 70), NSKT (SEQ ID NO: 71), NPQT (SEQ ID NO: 72), NAKT (SEQ ID NO: 73), LPIT (SEQ ID NO: 74), LAET (SEQ ID NO: 75), or NPQS (SEQ ID NO: 76). The invention encompasses embodiments in which ‘X’ in any sortase recognition motif disclosed herein or known in the art is amino acid, for example, any naturally-occurring or any non-naturally occurring amino acid. In some embodiments, X is selected from the 20 standard amino acids found most commonly in proteins found in living organisms. In some embodiments, e.g., where the recognition motif is LPXTG (SEQ ID NO: 78) or LPXT, X is D, E, A, N, Q, K, or R. In some embodiments, X in a particular recognition motif is selected from those amino acids that occur naturally at position 3 in a naturally occurring sortase substrate. For example, in some embodiments X is selected from K, E, N, Q, A in an LPXTG (SEQ ID NO: 78) or LPXT motif where the sortase is a sortase A. In some embodiments X is selected from K, S, E, L, A, N in an LPXTG (SEQ ID NO: 78) or LPXT motif and a class C sortase is used.

In some embodiments, a sortase recognition sequence further comprises one or more additional amino acids, e.g., at the N or C terminus. For example, one or more amino acids (e.g., up to 5 amino acids) having the identity of amino acids found immediately N-terminal to, or C-terminal to, a 5 amino acid recognition sequence in a naturally occurring sortase substrate may be incorporated. Such additional amino acids may provide context that improves the recognition of the recognition motif.

In some embodiments, a sortase recognition motif is masked. In contrast to an unmasked sortase recognition motif, which can be can be recognized by a sortase, a masked sortase recognition motif is a motif that is not recognized by a sortase but that can be readily modified (“unmasked”) such that the resulting motif is recognized by the sortase. For example, in some embodiments at least one amino acid of a masked sortase recognition motif comprises a side chain comprising a moiety that inhibits, e.g., prevents, recognition of the sequence by a sortase of interest, e.g., SrtAₐᵤᵣₑᵤₛ. Removal of the inhibiting moiety, in turn, allows recognition of the motif by the sortase. Masking may, for example, reduce recognition by at least 80%, 90%, 95%, or more (e.g., to undetectable levels) in certain embodiments. By way of example, in certain embodiments a threonine residue in a sortase recognition motif such as LPXTG (SEQ ID NO: 78) may be phosphorylated, thereby rendering it refractory to recognition and cleavage by SrtA. The masked recognition sequence can be unmasked by treatment with a phosphatase, thus allowing it to be used in a SrtA-catalyzed transamidation reaction.

The term “sortase substrate,” as used herein refers to any molecule that is recognized by a sortase, for example, any molecule that can partake in a sortase-mediated transpeptidation reaction. A typical sortase-mediated transpeptidation reaction involves a substrate comprising a C-terminal sortase recognition motif, e.g., an LPXTX motif, and a second substrate comprising an N-terminal sortase recognition motif, e.g., an N-terminal polyglycine or polyalanine. A sortase substrate may be a peptide or a protein, for example, a target protein on the surface of a virus, or a peptide comprising a sortase recognition motif such as an LPXTX motif or a polyglycine or polyalanine, wherein the peptide is conjugated to an agent, e.g., a small molecule, a binding agent, or a fluorophore. Accordingly, both proteins and non-protein molecules can be sortase substrates as long as they comprise a sortase recognition motif. Some examples of sortase substrates are described in more detail elsewhere herein and additional suitable sortase substrates will be apparent to the skilled artisan. The invention is not limited in this respect.

The term “sortagging,” as used herein, refers to the process of adding a tag, e.g., a moiety or molecule, for example, a protein, polypeptide, detectable label, binding agent, or click chemistry handle, onto a target molecule, for example, a target protein on the surface of a viral particle via a sortase-mediated transpeptidation reaction. Examples of additional suitable tags include, but are not limited to, amino acids, nucleic acids, polynucleotides, sugars, carbohydrates, polymers, lipids, fatty acids, and small molecules. Other suitable tags will be apparent to those of skill in the art and the invention is not limited in this aspect. In some embodiments, a tag comprises a sequence useful for purifying, expressing, solubilizing, and/or detecting a polypeptide. In some embodiments, a tag can serve multiple functions. In some embodiments, the tag is relatively small, e.g., ranging from a few amino acids up to about 100 amino acids long. In some embodiments, a tag is more than 100 amino acids long, e.g., up to about 500 amino acids long, or more. In some embodiments, a tag comprises an HA, TAP, Myc, 6×His, Flag, streptavidin, biotin, or GST tag, to name a few examples. In some embodiments, a tag comprises a solubility-enhancing tag (e.g., a SUMO tag, NUS A tag, SNUT tag, or a monomeric mutant of the Ocr protein of bacteriophage T7). See, e.g., Esposito D and Chatterjee D K. Curr Opin Biotechnol.; 17(4):353-8 (2006). In some embodiments, a tag is cleavable, so that it can be removed, e.g., by a protease. In some embodiments, this is achieved by including a protease cleavage site in the tag, e.g., adjacent or linked to a functional portion of the tag. Exemplary proteases include, e.g., thrombin, TEV protease, Factor Xa, PreScission protease, etc. In some embodiments, a “self-cleaving” tag is used. See, e.g., Wood et al., International PCT Application PCT/US2005/05763, filed on Feb. 24, 2005, and published as WO/2005/086654 on Sep. 22, 2005.

The term “target protein,” as used herein in the context of sortase-mediated modification of viral particles, refers to a protein on the surface of a virus that is the target of a sortase-mediated conjugation. For example, in an embodiment where M13 pIII is modified by sortagging, e.g., by adding a detectable label or a binding agent to M13 pIII on the surface of an M13 bacteriophage particle, pIII is the target protein. The term “target protein” may refer to a wild type or naturally occurring form of the respective protein, or to an engineered form, for example, to a recombinant protein variant comprising a sortase recognition motif not contained in a wild-type form of the protein. The term “modifying a target protein,” as used herein in the context of sortase-mediated protein modification, refers to a process of altering a target protein comprising a sortase recognition motif via a sortase-mediated transpeptidation reaction. Typically, the modifying results in the target protein being conjugated to an agent, for example, a peptide, protein, binding agent, detectable label, or small molecule.

The term “virus,” as used interchangeably herein with the term “viral particle,” refers to an infectious agent that can infect a living cell. A virus particle typically comprises the viral genome, e.g., as DNA, RNA, or a DNA/RNA hybrid, proteins associated with the viral genome that form a viral coat, and, in some cases an envelope of lipids that surrounds the viral protein coat. In some embodiments, a viral particle comprises a viral genome that can replicate inside a host cell once the virus has infected the cell. In some embodiments, the viral functions encoded in the viral genome result in the production of new viral particles by the host cell. In some embodiments, the newly generated viral particles can themselves infect additional host cells. Suitable viruses for use in the context of this invention typically comprise at least one surface protein comprising a sortase recognition motif. In some embodiments, the sortase recognition motif is comprised in a wild-type viral protein (e.g., a capsid protein or a viral surface protein). In some embodiments, the sortase recognition motif is encoded by a recombinant viral genome, e.g., a viral genome in which an open reading frame has been altered to insert a sortase recognition motif. A virus suitable for use according to aspects of this invention may be recombinant, and comprise genetic alterations other than the addition of a sortase recognition motif to a surface protein. For example, in some embodiment, a virus may be used that is replication-incompetent, or that carries in its genome a selectable marker, e.g., an antibiotic resistance marker, that can be used to identify cells infected by the virus. Viruses can be classified according to their genome structure and type of nucleic acid comprised in the respective viral particles. A suitable virus according to aspects of this invention may be a dsDNA virus comprising a double-stranded DNA genome (e.g. adenoviruses, herpesviruses, poxviruses), an ssDNA virus comprising a single-stranded DNA genome (e.g. parvoviruses), a dsRNA virus comprising a double-stranded RNA genome (e.g. reoviruses), a (+)ssRNA virus comprising a single stranded (+)sense strand RNA genome (e.g. picornaviruses, togaviruses), a (−)ssRNA virus comprising a single stranded (−)sense RNA (e.g. orthomyxoviruses, rhabdoviruses), an ssRNA-RT virus comprising a single-stranded (+)sense RNA with a DNA intermediate genome in its life-cycle that is generated by reverse transcription of the RNA genome (e.g. retroviruses), or a dsDNA-RT virus (e.g. hepadnaviruses). Exemplary viruses include, e.g., Retroviridae (e.g., lentiviruses such as human immunodeficiency viruses, such as HIV-I); Caliciviridae (e.g. strains that cause gastroenteritis); Togaviridae (e.g. equine encephalitis viruses, rubella viruses); Flaviridae (e.g. dengue viruses, encephalitis viruses, yellow fever viruses, hepatitis C virus); Coronaviridae (e.g. coronaviruses); Rhabdoviridae (e.g. vesicular stomatitis viruses, rabies viruses); Filoviridae (e.g. Ebola viruses); Paramyxoviridae (e.g. parainfluenza viruses, mumps virus, measles virus, respiratory syncytial virus); Orthomyxoviridae (e.g. influenza viruses); Bunyaviridae (e.g. Hantaan viruses, bunga viruses, phleboviruses and Nairo viruses); Arenaviridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), EBV, KSV); Poxyiridae (variola viruses, vaccinia viruses, pox viruses); and Picornaviridae (e.g. polio viruses, hepatitis A virus; enteroviruses, human coxsackie viruses, rhinoviruses, echoviruses). In some embodiments, the virus is a bacteriophage, for example, a bacteriophage belonging to the family of Myoviridae (e.g., T4 phage), Siphoviridae (e.g., k phage, Bacteriophage T5), Podoviridae (e.g., T7 phage), Ligamenvirales, Lipothrixviridae, Rudiviridae, Ampullaviridae, Bacilloviridae, Bicaudaviridae, Clavaviridae, Corticoviridae, Cystoviridae, Fuselloviridae, Globuloviridae, Guttavirus, Inoviridae, Leviviridae (e.g., MS2, Qβ), Microviridae (e.g., ΦX174), Plasmaviridae, or Tectiviridae. Exemplary suitable bacteriophages include, without limitation, Lambda phage (λ phage, lysogen), T2 phage, T4 phage, T7 phage, T12 phage, R17 phage, M13 phage, MS2 phage, G4 phage, P1 phage, Enterobacteria phage P2, P4 phage, ΦX174 phage, N4 phage, Φ6 phage, and Φ29 phage. Additional bacteriophages suitable for surface functionalization using methods, reagents, and kits provided herein will be apparent to those of skill in the art. Suitable bacteriophages include, for example, bacteriophages described in Stephen T. Abedon, The Bacteriophages, Oxford University Press, USA; 2^{nd} edition, Dec. 15, 2005, ISBN: 0195148509; particularly in parts III-V, pages 129-653; Elizabeth Kutter and Alexander Sulakvelidze: Bacteriophages: Biology and Applications. CRC Press; 1^{st} edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1^{st} edition (December, 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1^{st} edition (December 2008), ISBN: 1603275649; all of which are incorporated herein in their entirety by reference for disclosure of suitable phages and host cells as well as methods and protocols for isolation, culture, and manipulation of such phages.

In some embodiments, the phage is a filamentous phage. In some embodiments, the phage is an M13 phage. Wild-type M13 phage particles comprise a circular, single-stranded genome of approximately 6.4 kb. The wild-type genome includes ten genes, gI-gX, which, in turn, encode the ten M13 proteins, pI-pX, respectively. gVIII encodes pVIII, also often referred to as the major structural protein of the phage particles, while gIII encodes pIII, also referred to as the minor coat protein, which is required for infectivity of M13 phage particles. The M13 phage genome has extensively been studied and can be manipulated with recombinant techniques well known to those of skill in the art. For example, one or more of the wild-type genes can be deleted in whole or in part, and/or a heterologous nucleic acid construct can be inserted into the M13 genome. Such recombinant M13 phage genomes can be packaged into M13 phage particles in the presence of packaging proteins (e.g., pIII, pVI, pVII, pVIII, and pIX). The size of the M13 particles depends mainly on the size of the packaged genome. M13 does not have stringent genome size restrictions, and insertions of up to 42 kb have been reported. The M13 phage genome has been sequences, and M13 genomic sequences can be retrieved from public databases, such as the National Center for Biotechnology Information (NCBI) database (www.ncbi.nlm,nih.gov) and the ENSEMBL database (www.ensembl.org). An exemplary M13 genomic sequence is provided in entry V00604 of the National Center for Biotechnology Information (NCBI) database (www.ncbi.nlm,nih.gov):

>gi|56713234|emb|V00604.2| Phage M13 genome
(SEQ ID NO: 79)
AACGCTACTACTATTAGTAGAATTGATGCCACCTTTTCAGCTCGCGCCCCAAATGAAAATATAG
CTAAACAGGTTATTGACCATTTGCGAAATGTATCTAATGGTCAAACTAAATCTACTCGTTCGCA
GAATTGGGAATCAACTGTTACATGGAATGAAACTTCCAGACACCGTACTTTAGTTGCATATTTA
AAACATGTTGAGCTACAGCACCAGATTCAGCAATTAAGCTCTAAGCCATCCGCAAAAATGACCT
CTTATCAAAAGGAGCAATTAAAGGTACTCTCTAATCCTGACCTGTTGGAGTTTGCTTCCGGTCT
GGTTCGCTTTGAAGCTCGAATTAAAACGCGATATTTGAAGTCTTTCGGGCTTCCTCTTAATCTT
TTTGATGCAATCCGCTTTGCTTCTGACTATAATAGTCAGGGTAAAGACCTGATTTTTGATTTAT
GGTCATTCTCGTTTTCTGAACTGTTTAAAGCATTTGAGGGGGATTCAATGAATATTTATGACGA
TTCCGCAGTATTGGACGCTATCCAGTCTAAACATTTTACTATTACCCCCTCTGGCAAAACTTCT
TTTGCAAAAGCCTCTCGCTATTTTGGTTTTTATCGTCGTCTGGTAAACGAGGGTTATGATAGTG
TTGCTCTTACTATGCCTCGTAATTCCTTTTGGCGTTATGTATCTGCATTAGTTGAATGTGGTAT
TCCTAAATCTCAACTGATGAATCTTTCTACCTGTAATAATGTTGTTCCGTTAGTTCGTTTTATT
AACGTAGATTTTTCTTCCCAACGTCCTGACTGGTATAATGAGCCAGTTCTTAAAATCGCATAAG
GTAATTCACAATGATTAAAGTTGAAATTAAACCATCTCAAGCCCAATTTACTACTCGTTCTGGT
GTTTCTCGTCAGGGCAAGCCTTATTCACTGAATGAGCAGCTTTGTTACGTTGATTTGGGTAATG
AATATCCGGTTCTTGTCAAGATTACTCTTGATGAAGGTCAGCCAGCCTATGCGCCTGGTCTGTA
CACCGTTCATCTGTCCTCTTTCAAAGTTGGTCAGTTCGGTTCCCTTATGATTGACCGTCTGCGC
CTCGTTCCGGCTAAGTAACATGGAGCAGGTCGCGGATTTCGACACAATTTATCAGGCGATGATA
CAAATCTCCGTTGTACTTTGTTTCGCGCTTGGTATAATCGCTGGGGGTCAAAGATGAGTGTTTT
AGTGTATTCTTTCGCCTCTTTCGTTTTAGGTTGGTGCCTTCGTAGTGGCATTACGTATTTTACC
CGTTTAATGGAAACTTCCTCATGAAAAAGTCTTTAGTCCTCAAAGCCTCTGTAGCCGTTGCTAC
CCTCGTTCCGATGCTGTCTTTCGCTGCTGAGGGTGACGATCCCGCAAAAGCGGCCTTTAACTCC
CTGCAAGCCTCAGCGACCGAATATATCGGTTATGCGTGGGCGATGGTTGTTGTCATTGTCGGCG
CAACTATCGGTATCAAGCTGTTTAAGAAATTCACCTCGAAAGCAAGCTGATAAACCGATACAAT
TAAAGGCTCCTTTTGGAGCCTTTTTTTTTGGAGATTTTCAACATGAAAAAATTATTATTCGCAA
TTCCTTTAGTTGTTCCTTTCTATTCTCACTCCGCTGAAACTGTTGAAAGTTGTTTAGCAAAACC
CCATACAGAAAATTCATTTACTAACGTCTGGAAAGACGACAAAACTTTAGATCGTTACGCTAAC
TATGAGGGTTGTCTGTGGAATGCTACAGGCGTTGTAGTTTGTACTGGTGACGAAACTCAGTGTT
ACGGTACATGGGTTCCTATTGGGCTTGCTATCCCTGAAAATGAGGGTGGTGGCTCTGAGGGTGG
CGGTTCTGAGGGTGGCGGTTCTGAGGGTGGCGGTACTAAACCTCCTGAGTACGGTGATACACCT
ATTCCGGGCTATACTTATATCAACCCTCTCGACGGCACTTATCCGCCTGGTACTGAGCAAAACC
CCGCTAATCCTAATCCTTCTCTTGAGGAGTCTCAGCCTCTTAATACTTTCATGTTTCAGAATAA
TAGGTTCCGAAATAGGCAGGGGGCATTAACTGTTTATACGGGCACTGTTACTCAAGGCACTGAC
CCCGTTAAAACTTATTACCAGTACACTCCTGTATCATCAAAAGCCATGTATGACGCTTACTGGA
ACGGTAAATTCAGAGACTGCGCTTTCCATTCTGGCTTTAATGAGGATCCATTCGTTTGTGAATA
TCAAGGCCAATCGTCTGACCTGCCTCAACCTCCTGTCAATGCTGGCGGCGGCTCTGGTGGTGGT
TCTGGTGGCGGCTCTGAGGGTGGTGGCTCTGAGGGTGGCGGTTCTGAGGGTGGCGGCTCTGAGG
GAGGCGGTTCCGGTGGTGGCTCTGGTTCCGGTGATTTTGATTATGAAAAGATGGCAAACGCTAA
TAAGGGGGCTATGACCGAAAATGCCGATGAAAACGCGCTACAGTCTGACGCTAAAGGCAAACTT
GATTCTGTCGCTACTGATTACGGTGCTGCTATCGATGGTTTCATTGGTGACGTTTCCGGCCTTG
CTAATGGTAATGGTGCTACTGGTGATTTTGCTGGCTCTAATTCCCAAATGGCTCAAGTCGGTGA
CGGTGATAATTCACCTTTAATGAATAATTTCCGTCAATATTTACCTTCCCTCCCTCAATCGGTT
GAATGTCGCCCTTTTGTCTTTAGCGCTGGTAAACCATATGAATTTTCTATTGATTGTGACAAAA
TAAACTTATTCCGTGGTGTCTTTGCGTTTCTTTTATATGTTGCCACCTTTATGTATGTATTTTC
TACGTTTGCTAACATACTGCGTAATAAGGAGTCTTAATCATGCCAGTTCTTTTGGGTATTCCGT
TATTATTGCGTTTCCTCGGTTTCCTTCTGGTAACTTTGTTCGGCTATCTGCTTACTTTTCTTAA
AAAGGGCTTCGGTAAGATAGCTATTGCTATTTCATTGTTTCTTGCTCTTATTATTGGGCTTAAC
TCAATTCTTGTGGGTTATCTCTCTGATATTAGCGCTCAATTACCCTCTGACTTTGTTCAGGGTG
TTCAGTTAATTCTCCCGTCTAATGCGCTTCCCTGTTTTTATGTTATTCTCTCTGTAAAGGCTGC
TATTTTCATTTTTGACGTTAAACAAAAAATCGTTTCTTATTTGGATTGGGATAAATAATATGGC
TGTTTATTTTGTAACTGGCAAATTAGGCTCTGGAAAGACGCTCGTTAGCGTTGGTAAGATTCAG
GATAAAATTGTAGCTGGGTGCAAAATAGCAACTAATCTTGATTTAAGGCTTCAAAACCTCCCGC
AAGTCGGGAGGTTCGCTAAAACGCCTCGCGTTCTTAGAATACCGGATAAGCCTTCTATATCTGA
TTTGCTTGCTATTGGGCGCGGTAATGATTCCTACGATGAAAATAAAAACGGCTTGCTTGTTCTC
GATGAGTGCGGTACTTGGTTTAATACCCGTTCTTGGAATGATAAGGAAAGACAGCCGATTATTG
ATTGGTTTCTACATGCTCGTAAATTAGGATGGGATATTATTTTTCTTGTTCAGGACTTATCTAT
TGTTGATAAACAGGCGCGTTCTGCATTAGCTGAACATGTTGTTTATTGTCGTCGTCTGGACAGA
ATTACTTTACCTTTTGTCGGTACTTTATATTCTCTTATTACTGGCTCGAAAATGCCTCTGCCTA
AATTACATGTTGGCGTTGTTAAATATGGCGATTCTCAATTAAGCCCTACTGTTGAGCGTTGGCT
TTATACTGGTAAGAATTTGTATAACGCATATGATACTAAACAGGCTTTTTCTAGTAATTATGAT
TCCGGTGTTTATTCTTATTTAACGCCTTATTTATCACACGGTCGGTATTTCAAACCATTAAATT
TAGGTCAGAAGATGAAATTAACTAAAATATATTTGAAAAAGTTTTCTCGCGTTCTTTGTCTTGC
GATTGGATTTGCATCAGCATTTACATATAGTTATATAACCCAACCTAAGCCGGAGGTTAAAAAG
GTAGTCTCTCAGACCTATGATTTTGATAAATTCACTATTGACTCTTCTCAGCGTCTTAATCTAA
GCTATCGCTATGTTTTCAAGGATTCTAAGGGAAAATTAATTAATAGCGACGATTTACAGAAGCA
AGGTTATTCACTCACATATATTGATTTATGTACTGTTTCCATTAAAAAAGGTAATTCAAATGAA
ATTGTTAAATGTAATTAATTTTGTTTTCTTGATGTTTGTTTCATCATCTTCTTTTGCTCAGGTA
ATTGAAATGAATAATTCGCCTCTGCGCGATTTTGTAACTTGGTATTCAAAGCAATCAGGCGAAT
CCGTTATTGTTTCTCCCGATGTAAAAGGTACTGTTACTGTATATTCATCTGACGTTAAACCTGA
AAATCTACGCAATTTCTTTATTTCTGTTTTACGTGCTAATAATTTTGATATGGTTGGTTCAATT
CCTTCCATAATTCAGAAGTATAATCCAAACAATCAGGATTATATTGATGAATTGCCATCATCTG
ATAATCAGGAATATGATGATAATTCCGCTCCTTCTGGTGGTTTCTTTGTTCCGCAAAATGATAA
TGTTACTCAAACTTTTAAAATTAATAACGTTCGGGCAAAGGATTTAATACGAGTTGTCGAATTG
TTTGTAAAGTCTAATACTTCTAAATCCTCAAATGTATTATCTATTGACGGCTCTAATCTATTAG
TTGTTAGTGCACCTAAAGATATTTTAGATAACCTTCCTCAATTCCTTTCTACTGTTGATTTGCC
AACTGACCAGATATTGATTGAGGGTTTGATATTTGAGGTTCAGCAAGGTGATGCTTTAGATTTT
TCATTTGCTGCTGGCTCTCAGCGTGGCACTGTTGCAGGCGGTGTTAATACTGACCGCCTCACCT
CTGTTTTATCTTCTGCTGGTGGTTCGTTCGGTATTTTTAATGGCGATGTTTTAGGGCTATCAGT
TCGCGCATTAAAGACTAATAGCCATTCAAAAATATTGTCTGTGCCACGTATTCTTACGCTTTCA
GGTCAGAAGGGTTCTATCTCTGTTGGCCAGAATGTCCCTTTTATTACTGGTCGTGTGACTGGTG
AATCTGCCAATGTAAATAATCCATTTCAGACGATTGAGCGTCAAAATGTAGGTATTTCCATGAG
CGTTTTTCCTGTTGCAATGGCTGGCGGTAATATTGTTCTGGATATTACCAGCAAGGCCGATAGT
TTGAGTTCTTCTACTCAGGCAAGTGATGTTATTACTAATCAAAGAAGTATTGCTACAACGGTTA
ATTTGCGTGATGGACAGACTCTTTTACTCGGTGGCCTCACTGATTATAAAAACACTTCTCAAGA
TTCTGGCGTACCGTTCCTGTCTAAAATCCCTTTAATCGGCCTCCTGTTTAGCTCCCGCTCTGAT
TCCAACGAGGAAAGCACGTTATACGTGCTCGTCAAAGCAACCATAGTACGCGCCCTGTAGCGGC
GCATTAAGCGCGGCGGGTGTGGTGGTTACGCGCAGCGTGACCGCTACACTTGCCAGCGCCCTAG
CGCCCGCTCCTTTCGCTTTCTTCCCTTCCTTTCTCGCCACGTTCGCCGGCTTTCCCCGTCAAGC
TCTAAATCGGGGGCTCCCTTTAGGGTTCCGATTTAGTGCTTTACGGCACCTCGACCCCAAAAAA
CTTGATTTGGGTGATGGTTCACGTAGTGGGCCATCGCCCTGATAGACGGTTTTTCGCCCTTTGA
CGTTGGAGTCCACGTTCTTTAATAGTGGACTCTTGTTCCAAACTGGAACAACACTCAACCCTAT
CTCGGGCTATTCTTTTGATTTATAAGGGATTTTGCCGATTTCGGCCTATTGGTTAAAAAATGAG
CTGATTTAACAAAAATTTAACGCGAATTTTAACAAAATATTAACGTTTACAATTTAAATATTTG
CTTATACAATCTTCCTGTTTTTGGGGCTTTTCTGATTATCAACCGGGGTACATATGATTGACAT
GCTAGTTTTACGATTACCGTTCATCGATTCTCTTGTTTGCTCCAGACTCTCAGGCAATGACCTG
ATAGCCTTTGTAGACCTCTCAAAAATAGCTACCCTCTCCGGCATGAATTTATCAGCTAGAACGG
TTGAATATCATATTGATGGTGATTTGACTGTCTCCGGCCTTTCTCACCCTTTTGAATCTTTACC
TACACATTACTCAGGCATTGCATTTAAAATATATGAGGGTTCTAAAAATTTTTATCCTTGCGTT
GAAATAAAGGCTTCTCCCGCAAAAGTATTACAGGGTCATAATGTTTTTGGTACAACCGATTTAG
CTTTATGCTCTGAGGCTTTATTGCTTAATTTTGCTAATTCTTTGCCTTGCCTGTATGATTTATT
GGATGTT
GENE II: join(6006 . . . 6407, 1 . . . 831)
(SEQ ID NO: 80)
translation = MIDMLVLRLPFIDSLVCSRLSGNDLIAFVDLSKIATLSGMNLSARTVEYHID
GDLTVSGLSHPFESLPTHYSGIAFKIYEGSKNFYPCVEIKASPAKVLQGHNVFGTTDLALCSEA
LLLNFANSLPCLYDLLDVNATTISRIDATFSARAPNENIAKQVIDHLRNVSNGQTKSTRSQNWE
STVTWNETSRHRTLVAYLKHVELQHQIQQLSSKPSAKMTSYQKEQLKVLSNPDLLEFASGLVRF
EARIKTRYLKSFGLPLNLFDAIRFASDYNSQGKDLIFDLWSFSFSELFKAFEGDSMNIYDDSAV
LDAIQSKHFTITPSGKTSFAKASRYFGFYRRLVNEGYDSVALTMPRNSFWRYVSALVECGIPKS
QLMNLSTCNNVVPLVRFINVDFSSQRPDWYNEPVLKIA
GENE X (encoding pX): 496 . . . 831
(SEQ ID NO: 81)
translation = MNIYDDSAVLDAIQSKHFTITPSGKTSFAKASRYFGFYRRLVNEGYDSVALT
MPRNSFWRYVSALVECGIPKSQLMNLSTCNNVVPLVRFINVDFSSQRPDWYNEPVLKIA
GENE V (encoding pV): 843 . . . 1106
(SEQ ID NO: 82)
translation = MIKVEIKPSQAQFTTRSGVSRQGKPYSLNEQLCYVDLGNEYPVLVKITLDEG
QPAYAPGLYTVHLSSFKVGQFGSLMIDRLRLVPAK
GENE VII (encoding pVII): 1108 . . . 1209
(SEQ ID NO: 83)
translation = MEQVADFDTIYQAMIQISVVLCFALGIIAGGQR
GENE IX (encoding pIX): 1206 . . . 1304
(SEQ ID NO: 84)
translation = ″MSVLVYSFASFVLGWCLRSGITYFTRLMETSS
GENE VIII (encoding pVIII): 1301 . . . 1522
(SEQ ID NO: 85)
translation = MKKSLVLKASVAVATLVPMLSFAAEGDDPAKAAFNSLQASATEYIGYAWAMV
VVIVGATIGIKLFKKFTSKAS
GENE III (encoding pIII): 1579 . . . 2853
(SEQ ID NO: 86)
translation = MKKLLFAIPLVVPFYSHSAETVESCLAKPHTENSFTNVWKDDKILDRYANYE
GCLWNATGVVVCTGDETQCYGTWVPIGLAIPENEGGGSEGGGSEGGGSEGGGTKPPEYGDTPIP
GYTYINPLDGTYPPGTEQNPANPNPSLEESQPLNTFMFQNNRFRNRQGALTVYTGTVTQGTDPV
KTYYQYTPVSSKAMYDAYWNGKFRDCAFHSGFNEDPFVCEYQGQSSDLPQPPVNAGGGSGGGSG
GGSEGGGSEGGGSEGGGSEGGGSGGGSGSGDFDYEKMANANKGAMTENADENALQSDAKGKLDS
VATDYGAAIDGFIGDVSGLANGNGATGDFAGSNSQMAQVGDGDNSPLMNNFRQYLPSLPQSVEC
RPFVFSAGKPYEFSIDCDKINLFRGVFAFLLYVATFMYVFSTFANILRNKES
GENE VI (encoding pVI): 2856 . . . 3194
(SEQ ID NO: 87)
translation = MPVLLGIPLLLRFLGFLLVTLFGYLLTFLKKGFGKIAIAISLFLALIIGLNS
ILVGYLSDISAQLPSDFVQGVQLILPSNALPCFYVILSVKAAIFIFDVKQKIVSYLDWDK
GENE I (encoding pI): 3196 . . . 4242
(SEQ ID NO: 88)
translation = MAVYFVTGKLGSGKTLVSVGKIQDKIVAGCKIATNLDLRLQNLPQVGRFAKT
PRVLRIPDKPSISDLLAIGRGNDSYDENKNGLLVLDECGTWFNTRSWNDKERQPIIDWFLHARK
LGWDIIFLVQDLSIVDKQARSALAEHVVYCRRLDRITLPFVGTLYSLITGSKMPLPKLHVGVVK
YGDSQLSPTVERWLYTGKNLYNAYDTKQAFSSNYDSGVYSYLTPYLSHGRYFKPLNLGQKMKLT
KIYLKKFSRVLCLAIGFASAFTYSYITQPKPEVKKVVSQTYDFDKFTIDSSQRLNLSYRYVFKD
SKGKLINSDDLQKQGYSLTYIDLCTVSIKKGNSNEIVKCN
GENE IV (encoding pIV): 4220 . . . 5500
(SEQ ID NO: 89)
translation = MKLLNVINFVFLMFVSSSSFAQVIEMNNSPLRDFVTWYSKQSGESVIVSPDV
KGTVTVYSSDVKPENLRNFFISVLRANNFDMVGSIPSIIQKYNPNNQDYIDELPSSDNQEYDDN
SAPSGGFFVPQNDNVTQTFKINNVRAKDLIRVVELFVKSNTSKSSNVLSIDGSNLLVVSAPKDI
LDNLPQFLSTVDLPTDQILIEGLIFEVQQGDALDFSFAAGSQRGTVAGGVNTDRLTSVLSSAGG
SFGIFNGDVLGLSVRALKTNSHSKILSVPRILTLSGQKGSISVGQNVPFITGRVTGESANVNNP
FQTIERQNVGISMSVFPVAMAGGNIVLDITSKADSLSSSTQASDVITNQRSIATTVNLRDGQTL
LLGGLTDYKNTSQDSGVPFLSKIPLIGLLFSSRSDSNEESTLYVLVKATIVRAL

The term “viral capsid,” as used herein, refers to a protein coat, also sometimes referred to as a protein shell, of a virus. The viral capsid encloses the viral genetic material. The capsid of most viruses comprises a plurality of oligomeric structural subunits made of proteins called protomers. The observable 3-dimensional morphological subunits, which may or may not correspond to individual proteins, are called capsomeres. Viral capsids can be classified according to their structure, e.g., into helical and icosahedral capsids. Some viruses, e.g., bacteriophages, have developed more complicated structures. Some viral capsids are enveloped with a lipid membrane known as the viral envelope, which is typically acquired by the capsid from a membrane of the host cell.

### DETAILED DESCRIPTION OF CERTAIN EMBODIMENTS

This invention is based, at least in part, on the recognition that sortases can be exploited to conjugate a variety of moieties to the proteins on the surface of viruses, for example, to the capsid proteins of M13 bacteriophage. Such sortase-mediated conjugation approaches can be used to confer new functions to viral particles. For example, the conjugation of a detectable label allows for the isolation and/or quantification of viral particles and can also be used to label cells bound or infected by the viral particles. For another example, sortase-mediated conjugation of binding moieties, for example, of antibodies or antibody fragments, nucleic acids, or of biotin and streptavidin, can be used to confer new binding properties to viral particles, e.g., in order to generate complex structures of associated, e.g., concatenated, viral particles.

Some aspects of this disclosure provide methods, reagents, and kits that can be used to functionalize proteins on the surface of viruses, for example, by conjugating such proteins to a molecule or a plurality of molecules conferring a desired function. Examples of such molecules include, without limitation, detectable labels, small molecules, and binding agents. The sortase-mediated techniques described herein allow for functionalization of viral surface proteins with high specificity and with efficiencies that surpass those of any known recombinant techniques, such as methods used in the context of phage display technology. Another advantage of the methods, reagents, and kits provided herein is that agents (e.g., proteins, binding agents, or small molecules) can be conjugated to viral surface proteins that cannot be genetically encoded, e.g., because of size limitations for insertions into the viral gene or genome encoding a target viral protein to be modified, or because the agent is not a gene product that can be encoded by the viral genome.

For example, capsid proteins (e.g., pIII, pIX, and pVIII) of bacteriophage M13 can be functionalized, according to some aspects of this disclosure, with entities ranging from small molecules (e.g., fluorophores, biotin) to folded proteins (e.g., GFP, antibodies, streptavidin) in a site-specific manner and with yields that surpass those of any reported using phage display technology. A non-limiting example of phage protein modification according to some aspects of this disclosure is the sortase-mediated modification of pVIII, which is difficult to modify with conventional approaches of genetic engineering or chemical labeling. While a phage vector limits the size of an insert into pVIII to a few amino acids, a phagemid system limits the number of copies actually displayed on the surface of M13 phage. Using sortase-based reactions, a 100-fold increase in the efficiency of display of GFP onto pVIII is achieved, as described in more detail elsewhere herein.

Taking advantage of orthogonal sortases, a plurality of viral capsid proteins can be modified in the same viral particle while maintaining excellent specificity of labeling. The methods provided herein are simple and effective for creating a variety of structures on the surface of viral particles, e.g., of M13 phage capsid proteins.

The methods, reagents, and kits provided herein can be used to generate complex, virus-templated structures, e.g., branched concatemers, such as lampbrush structures, that can be engineered to carry out novel functions, e.g., structural functions or the harvesting of light. The methods, reagents, and kits provided herein allow for the use of biological structures, e.g., viral particles, as building blocks for the engineering of new materials and structures and for the functionalization of the surface of such structures. The methods, reagents, and kits provided herein can also be used to engineer new functionalities into viral particles, for example, the binding of a new spectrum of cells, the interaction with a specific target protein, e.g., a specific receptor on the surface of a cell of interest, or the delivery of a payload to a specific type of cell expressing a surface molecule of interest. Viral particles can be functionalized using the strategies disclosed herein to attach a cell targeting motif, e.g., a binding agent such as an antibody, nucleic acid, or a bacterial toxin, to the viral surface, in order to increase the uptake/internalization of the functionalized virus by a specific cell or cell type. In some embodiments, the methods and strategies disclosed herein can be used to generate a viral particle that can bind and deliver its genome to a previously uninfectable host cell, resulting in expression of a viral gene product in the host cell. The strategies and methods disclosed herein can also be used to attach a payload, e.g., a functional protein or a small molecule to the surface of a virus that can be delivered upon entry into a target cell.

The strategies, methods, reagents, and kits disclosed herein can also be used to improve the identification of binding targets in phage display libraries, for example, by using fluorescently labeled phage for the detection of binding events; to generate functionalized viral particles for use as a handle in single molecule force spectroscopy experiments, allowing, for example, to post-translationally attach properly folded complex proteins to the surface of a viral particle; to create complex structures comprising viral particles functionalized with binding agents as building blocks, e.g., using connections between specific viral capsid proteins; to target viral particles to specific cells; and to deliver payloads to target cells upon binding or infection, e.g., toxic agents such as plant or bacterial toxins, antibiotics, and drugs.

### Sortase-Mediated Functionalization of Viral Capsid Proteins

The present invention provides methods, reagents, and kits for the functionalization of viral capsid proteins. Typically, a method of functionalizing a viral capsid protein as provided herein comprises conjugating the target capsid protein with an agent via a sortase-mediated transpeptidation reaction. In order for a sortase-mediated transpeptidation to be possible, both the target protein and the agent must be recognized by the sortase and must be capable of acting as a substrate of the sortase in the transpeptidation reaction. Accordingly, the methods for functionalization of viral capsid proteins provided herein involve viral proteins and agents that comprise or are conjugated to a sortase recognition motif. Some viral proteins and some agents (e.g., proteins) may comprise a suitable sortase recognition motif. However, in some embodiments, the target protein and/or the agent is engineered to comprise a suitable sortase recognition motif, for example, via protein engineering (e.g., using recombinant technologies) or via chemical synthesis (e.g., linking a non-protein agent to a sortase recognition motif).

Typically, a method for viral capsid protein functionalization as provided herein comprises contacting a target protein, e.g., a viral capsid protein comprising a sortase recognition motif that is accessible on the surface of a viral particle, with an agent comprising a sortase recognition motif, in the presence of a sortase under conditions suitable for the sortase to conjugate the target protein to the agent via a sortase-mediated transpeptidation reaction.

For example, some embodiments provide methods for modifying a target protein, for example, a target viral capsid protein, comprising a sortase recognition motif on the surface of a virus, that includes contacting the target protein with a sortase substrate conjugated to an agent in the presence of a sortase under conditions suitable for the sortase to ligate the sortase substrate to the target protein. In some embodiments, the target protein comprises an N-terminal sortase recognition motif, and the sortase substrate conjugated to the agent comprises a C-terminal sortase recognition motif. In other embodiments, the target protein comprises a C-terminal sortase recognition motif, and the sortase substrate conjugated to the agent comprises an N-terminal sortase recognition motif. The C- and N-terminal recognition motif are recognized as substrates by the sortase being employed and ligated in a transpeptidation reaction.

In a given embodiment, whether a viral target protein comprises (e.g., is engineered to comprise) a C-terminal or an N-terminal sortase recognition motif will depend on the accessibility of the C-terminus and/or the N-terminus of the target protein on the surface of the virus. For example, if the C-terminus of the target protein is accessible on the surface of the virus, e.g., on the surface of the viral capsid, and the N-terminus is not, then a C-terminal sortase recognition motif is suitable and vice versa. For example, in some embodiments, an M13 phage is provided that comprises a pIII protein containing an N-terminal sortase recognition motif, e.g., an N-terminal polyglycine sequence, and is functionalized at the N-terminus by contacting it with a sortase substrate comprising a C-terminal sortase recognition motif, e.g., an LPETG (SEQ ID NO: 10) sequence, conjugated to an agent, e.g., GFP, in the presence of a sortase, e.g., a SrtAₐᵤᵣₑᵤₛ, under suitable conditions for the sortase to conjugate pIII and GFP via a sortase-mediated transpeptidation reaction.

Whether the C-terminus and/or the N-terminus of a given viral target protein is accessible or not on the surface of the respective virus will be apparent to those of skill in the art. Many viruses have been sequenced and the structures of the respective viral capsids have been investigated and can be accessed in publicly available databases, such as ENSEMBL (www.ensembl.org) and NCBI (www.ncbi.nlm.nih.gov). Where structural data is lacking, those of skill in the art will be able to determine the accessibility of the C-terminus and/or the N-terminus of a given viral protein on the surface of the respective viral capsid with no more than routine experimentation.

In some embodiments, methods are provided that allow for the functionalization, or sortagging, of a plurality of different viral proteins of a virus. For example, in some embodiments, a method is provided that allows for the functionalization of 2, 3, 4, 5, 6, 7, 8, 9, or different viral proteins. In some embodiments, specific functionalization of a plurality of viral capsid proteins involves the use of different sortases, each specifically recognizing a different sortase recognition motif. For example, in some embodiments, a first target protein is functionalized with SrtAₐᵤᵣₑᵤₛ, recognizing the C-terminal sortase recognition motif LPETGG (SEQ ID NO: 13) and the N-terminal sortase recognition motif (G)ₙ, and a second target protein is functionalized with SrtA_{pyogenes}, recognizing the C-terminal sortase recognition motif LPETAA (SEQ ID NO: 12) and the N-terminal sortase recognition motif (A)ₙ. The sortases in this example recognize their respective recognition motif but do not recognize the other sortase recognition motif to a significant extent, and, thus, “specifically” recognize their respective recognition motif. In some embodiments, a sortase binds a sortase recognition motif specifically if it binds the motif with an affinity that is at least 5-fold, 10-fold, 20-fold, 50-fold, 100-fold, 200-fold, 500-fold, 1000-fold, or more than 1000-fold higher than the affinity that the sortase binds a different motif. Such a pairing of orthogonal sortases and their respective recognition motifs, e.g., of the orthogonal sortase A enzymes SrtAₐᵤᵣₑᵤₛ and SrtA_{pyogenes}, can be used to site-specifically conjugate two different moieties onto two different capsid proteins (e.g., a first binding agent to pIII and a second binding agent to pVIII of M13 bacteriophage particles). In some embodiments, sortagging of a plurality of different proteins is achieved by sequentially contacting a virus comprising the different proteins with a first sortase recognizing a sortase recognition motif of a first target protein and a suitable first sortase substrate, and then with a second sortase recognizing a sortase recognition motif of a second target protein and a second suitable sortase substrate, and so forth. Alternatively, the virus may be contacted with a plurality of sortases in parallel, for example, with a first sortase recognizing a sortase recognition motif of a first target protein and a suitable first sortase substrate, and with a second sortase recognizing a sortase recognition motif of a second target protein and a second suitable sortase substrate, and so forth. It will be understood by those of skill in the art, that suitable orthogonal sortases preferentially recognize their own motifs over the motifs of other sortases, but that a basal level of recognition of other sortase recognition motifs is not detrimental. For example, SrtA_{pyogenes} is able to recognize an LPXTG (SEQ ID NO: 78) motif, but strongly prefers an LPXTA (SEQ ID NO: 91) motif, while SrtAₐᵤᵣₑᵤₛ shows no cleavage activity for the LPXTA (SEQ ID NO: 91) motif. These two sortases are suitable orthogonal sortases according to some aspects of this invention, as are sortases that exclusively recognize their own sortase recognition sequence.

For example, in some embodiments, a first viral target protein, e.g., M13 pIII comprising an N-terminal poly-G sequence, is functionalized using sortase A from Staphylococcus aureus (SrtAₐᵤᵣₑᵤₛ), and a second target protein, e.g., M13 pVIII comprising an N-terminal poly-A sequence, is functionalized using sortase A from Streptococcus pyogenes (SrtA_{pyogenes}). In some such embodiments, the virus, e.g., the M13 phage, may be contacted first with SrtAₐᵤᵣₑᵤₛ (and a suitable substrate) and subsequently with SrtA_{pyogenes} (and a suitable substrate), or, since the two sortases are orthogonal sortases, the respective virus may be contacted with both sortases and both substrates at the same time.

Any sortases that recognize sufficiently different sortase recognition motifs with sufficient specificity are suitable for sortagging of a plurality of viral proteins of the same virus. The respective sortase recognition motifs can be inserted into the target proteins using recombinant technologies known to those of skill in the art. In some embodiments, suitable sortase recognition motifs may be present in a wild type target protein, for example, an N-terminal polyglycine or polyalanine sequence, in which case no further engineering of the target protein may be required. The skilled artisan will understand that the choice of a suitable sortase for the functionalization of a given target protein may depend on the sequence of the target protein, e.g., on whether or not the target protein comprises a sequence at its C-terminus or its N-terminus that can be recognized as a substrate by any known sortase. In some embodiments, use of a sortase that recognizes a naturally-occurring C-terminal or N-terminal recognition motif is preferred since further engineering of the target protein can be avoided.

In some embodiments, a plurality of different target proteins is functionalized on the surface of the same viral particle. In some embodiments, the different target proteins are functionalized with different agents. For example, in some embodiments, a first target protein may be functionalized with a first binding agent, and a second target protein may be functionalized with a second binding agent. One example of such an embodiment is the functionalization of M13 pIII with biotin and the functionalization of M13 pVIII with streptavidin on the surface of the same M13 phage particle. Another example of such an embodiment is the functionalization of M13 pIII with a nucleic acid molecule, e.g., an oligonucleotide, and the functionalization of M13 VIII with a different nucleic acid molecule, e.g., a different oligonucleotide. For another example, in some embodiments, a first target protein is functionalized with a binding agent, and a second target protein is functionalized with a detectable label. In some embodiments, a first target protein is functionalized with a binding agent, a second target protein is functionalized with a detectable label, and a third target protein is functionalized with a click chemistry handle. Additional embodiments in which a plurality of different target proteins is sortagged with a plurality of different agents are provided herein, and further embodiments will be apparent to those of skill in the art based on the present disclosure. It will be understood that the invention is not limited in the number of different target proteins to be functionalized nor the number of different agents to be conjugated to the target proteins.

In some embodiments, an engineered viral capsid protein provided herein comprises a sortase recognition motif, e.g., a C-terminal or an N-terminal sortase recognition motif, within a loop structure. In some embodiments, the loop structure is formed by disulfide bonds between two cysteine residues flanking the sortase recognition motif. In some embodiments, the loop structure is situated at the N-terminus or the C-terminus of the engineered viral capsid protein, or inserted into the sequence of the viral capsid protein near the N- or the C-terminus (e.g., 1, 2, 3, 4, 5, 6, 7, 8, 9, 10, less than 15, less than 20, or less than 25 amino acid residues away from the N- or C-terminus of the viral capsid protein). In some embodiments, the loop structure comprises a cleavable site or a cleavable bond, the cleavage of which opens the loop. In some embodiments, the cleavable bond is a photocleavable bond. In some embodiments, the cleavable bond is a peptide bond, e.g., a peptide bond situated in a protease cleavage site comprised in the loop structure. In some embodiments, the loop structure comprises a protease cleavage site situated between the cysteine residues forming the loop and is, thus, sensitive to cleavage by the protease. In some embodiments, cleavage of the engineered viral capsid protein by the protease opens the loop structure. In some embodiments, the loop structure comprises an N-terminal cysteine, a sortase recognition motif situated C-terminally of the N-terminal cysteine, a protease cleavage site situated C-terminally of the sortase recognition motif, and a C-terminal cysteine. In some embodiments, the loop structure comprises an N-terminal cysteine, a protease cleavage site situated C-terminally of the N-terminal cysteine, a sortase recognition motif situated C-terminally of the protease cleavage site, and a C-terminal cysteine. In some embodiments, an amino acid residue, sequence, or structure comprised in the loop structure (e.g., the N-terminal cysteine, sortase recognition motif, protease cleavage site, and C-terminal cysteine) may be conjugated to another residue, sequence or structure of the loop via a linker, e.g., an amino acid or peptide linker. In some embodiments, the linker is a cleavable linker. In some embodiments, the linker is 3, 4, 5, 6, 7, 8, 9, or 10 amino acid residues long. In some embodiments, the linker comprises more than 10 amino acids. Suitable protease cleavage sites (and corresponding proteases cleaving such sites) are described herein. Exemplary suitable cleavage sites and corresponding proteases include, e.g., thrombin, TEV protease, Factor Xa, PreScission protease, and papain cleavage sites. Additional suitable proteases and cleavage sites will be apparent to the skilled artisan, and such suitable proteases and cleavage sites include, without limitation, those reported in the passage from paragraph [0093] to paragraph [0097], and in Table 2 and the Table following paragraph [0097] of U.S. patent application Ser. No. 13/642,458, publication number US2013/0122043, by Guimaraes and Ploegh, the entire contents of which passage and tables are incorporated herein by reference. In some embodiments, the loop structure comprises a bacterial toxin sequence, e.g., a sequence of a bacterial protein that comprises a loop structure. Exemplary suitable bacterial toxin sequences are described herein, and additional suitable sequences will be apparent to those of skill in the art based on the instant disclosure. Such suitable sequences include, without limitation, those reported in the passage from paragraph [0044] to paragraph [0080] and in paragraph [0175] of U.S. patent application Ser. No. 13/642,458, publication number US2013/0122043, by Guimaraes and Ploegh, the entire contents of which passage and paragraph are incorporated herein by reference. Exemplary suitable loop structures that are useful for engineering viral capsid proteins are disclosed herein, and additional suitable loop structures will be apparent to those of skill in the art. Such additional loop structures include, for example, those reported in U.S. patent application, U.S. Ser. No. 13/642,458, publication number US2013/0122043, by Guimaraes and Ploegh, the entire contents of which are incorporated herein by reference.

Sortases, sortase-mediated transacylation reactions, and their use in transpeptidation (sometimes also referred to as transacylation) for protein engineering are well known to those of skill in the art (see, e.g., Ploegh et al., International PCT Patent Application, PCT/US2010/000274, filed Feb. 1, 2010, published as WO 2010/087994 on Aug. 5, 2010, and Ploegh et al., International PCT Patent Application PCT/US2011/033303, filed Apr. 20, 2011, published as WO 2011/133704 on Oct. 27, 2011, the entire contents of which are incorporated herein by reference). In general, the transpeptidation reaction catalyzed by sortase results in the conjugation of a protein containing a C-terminal sortase recognition motif e.g., LPXTX (wherein each occurrence of X independently represents any amino acid residue), with a peptide comprising an N-terminal sortase recognition motif, e.g., one or more N-terminal glycine residues. In some embodiments, the sortase recognition motif is a sortase recognition motif described herein. In certain embodiments, the sortase recognition motif is LPXT motif or LPXTG (SEQ ID NO: 78).

The sortase transacylation reaction provides means for efficiently linking an acyl donor with a nucleophilic acyl acceptor. This principle is widely applicable to many acyl donors and a multitude of different acyl acceptors. Previously, the sortase reaction was employed for ligating proteins and/or peptides to one another, ligating synthetic peptides to recombinant proteins, linking a reporting molecule to a protein or peptide, joining a nucleic acid to a protein or peptide, conjugating a protein or peptide to a solid support or polymer, and linking a protein or peptide to a label. Such products and processes save cost and time associated with ligation product synthesis and are useful for conveniently linking an acyl donor to an acyl acceptor. However, the modification and functionalization of proteins on the surface of viral particles via sortagging, as provided herein, has not been described previously.

Sortase-mediated transpeptidation reactions (also sometimes referred to as transacylation reactions) are catalyzed by the transamidase activity of sortase, which forms a peptide linkage (an amide linkage), between an acyl donor compound and a nucleophilic acyl acceptor containing an NH₂—CH₂-moiety. In some embodiments, the sortase employed to carry out a sortase-mediated transpeptidation reaction is sortase A (SrtA). However, it should be noted that any sortase, or transamidase, catalyzing a transacylation reaction can be used in some embodiments of this invention, as the invention is not limited to the use of sortase A.

In certain embodiments, a sortase-mediated transpeptidation reaction for C-terminal functionalization of a viral surface protein, for example, of an M13 capsid protein, is provided that comprises a step of contacting a virus comprising a surface protein comprising a C-terminal sortase recognition sequence of the structure:

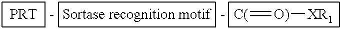

wherein
PRT is a viral capsid protein;
the sortase recognition motif is a C-terminal sortase recognition motif, e.g., an LP(Xaa)T motif, wherein Xaa represents any amino acid residue;
X is —O—, —NR—, or —S—; wherein R is hydrogen, substituted or unsubstituted aliphatic, or substituted or unsubstituted heteroaliphatic;
R¹ is H, acyl, substituted or unsubstituted aliphatic, substituted or unsubstituted heteroaliphatic, substituted or unsubstituted aryl, or substituted or unsubstituted heteroaryl;

with a nucleophilic moiety conjugated to an agent, according to the formula:

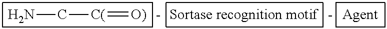

wherein
the sortase recognition motif is an N-terminal sortase recognition motif, for example, a polyglycine (Gₙ) or polyalanine (Aₙ) motif (wherein n is an integer between 0-100 inclusive);
the agent is acyl, substituted or unsubstituted aliphatic, substituted or unsubstituted heteroaliphatic, substituted or unsubstituted aryl, substituted or unsubstituted heteroaryl, an amino acid, a peptide, a protein, a polynucleotide, a carbohydrate, a tag, a metal atom, a contrast agent, a catalyst, a non-polypeptide polymer, a synthetic polymer, a recognition element, a small molecule, a lipid, a linker, or a label; and
the nucleophilic compound comprises, optionally, a linker connecting the agent to the nucleophilic amine group;

in the presence of a sortase, under conditions suitable to form a functionalized viral surface protein of formula:

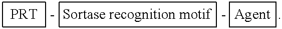

In certain embodiments, a sortase-mediated transpeptidation reaction for N-terminal functionalization of a viral surface protein, for example, of an M13 capsid protein, is provided that comprises a step of contacting a virus comprising a surface protein comprising an N-terminal sortase recognition sequence of the structure:

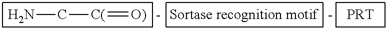

wherein
PRT is a viral capsid protein;
the sortase recognition motif is an N-terminal sortase recognition motif, for example, a polyglycine (Gₙ) or polyalanine (Aₙ) motif (wherein n is an integer between 0-100 inclusive);
   with an agent conjugated to a C-terminal sortase recognition motif, of the formula:

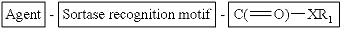

wherein
the agent is acyl, substituted or unsubstituted aliphatic, substituted or unsubstituted heteroaliphatic, substituted or unsubstituted aryl, substituted or unsubstituted heteroaryl, an amino acid, a peptide, a protein, a polynucleotide, a carbohydrate, a tag, a metal atom, a contrast agent, a catalyst, a non-polypeptide polymer, a synthetic polymer, a recognition element, a small molecule, a lipid, a linker, or a label;
optionally, wherein the agent is connected to the nucleophilic amine group via a linker;
the sortase recognition motif is a C-terminal sortase recognition motif, e.g., an LP(Xaa)T motif, wherein Xaa represents any amino acid residue;
X is —O—, —NR—, or —S—; wherein R is hydrogen, substituted or unsubstituted aliphatic, or substituted or unsubstituted heteroaliphatic; and
R¹ is H, acyl, substituted or unsubstituted aliphatic, substituted or unsubstituted heteroaliphatic, substituted or unsubstituted aryl, or substituted or unsubstituted heteroaryl;

in the presence of a sortase, under conditions suitable to form a functionalized viral surface protein of formula:

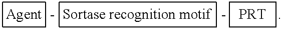

In some embodiments, the C-terminal sortase recognition motif is LPXT, wherein X is a standard or non-standard amino acid. In some embodiments, X is selected from D, E, A, N, Q, K, or R. In some embodiments, the recognition sequence is selected from LPXT, LPXT, SPXT, LAXT, LSXT, NPXT, VPXT, IPXT, and YPXR. In some embodiments, X is selected to match a naturally occurring transamidase recognition sequence. In some embodiments, the transamidase recognition sequence is selected from LPKT (SEQ ID NO: 93), LPIT (SEQ ID NO: 94), LPDT (SEQ ID NO: 95), SPKT (SEQ ID NO: 96), LAET (SEQ ID NO: 97), LAAT (SEQ ID NO: 98), LAET (SEQ ID NO: 99), LAST (SEQ ID NO: 100), LAET (SEQ ID NO: 101), LPLT (SEQ ID NO: 102), LSRT (SEQ ID NO: 103), LPET (SEQ ID NO: 104), VPDT (SEQ ID NO: 105), IPQT (SEQ ID NO: 106), YPRR (SEQ ID NO: 107), LPMT (SEQ ID NO: 108), LPLT (SEQ ID NO: 109), LAFT (SEQ ID NO: 110), LPQT (SEQ ID NO: 111), NSKT (SEQ ID NO: 112), NPQT (SEQ ID NO: 113), NAKT (SEQ ID NO: 114), and NPQS (SEQ ID NO: 115). In some embodiments, e.g., in certain embodiments in which sortase A is used, the transamidase recognition motif comprises the amino acid sequence X₁PX₂X₃, where X₁ is leucine, isoleucine, valine, or methionine; X₂ is any amino acid; X₃ is threonine, serine, or alanine; P is proline and G is glycine. In specific embodiments, as noted above, X₁ is leucine and X₃ is threonine. In certain embodiments, X₂ is aspartate, glutamate, alanine, glutamine, lysine, or methionine. In certain embodiments, e.g., where sortase B is utilized, the recognition sequence often comprises the amino acid sequence NPX₁TX₂, where X₁ is glutamine or lysine; X₂ is asparagine or glycine; N is asparagine; P is proline, and T is threonine. The invention encompasses the recognition that selection of X may be based at least in part in order to confer desired properties on the compound containing the recognition motif. In some embodiments, X is selected to modify a property of the compound that contains the recognition motif, such as to increase or decrease solubility in a particular solvent. In some embodiments, X is selected to be compatible with reaction conditions to be used in synthesizing a compound comprising the recognition motif, e.g., to be unreactive towards reactants used in the synthesis. One of ordinary skill will appreciate that, in certain embodiments, the C-terminal amino acid of the C-terminal sortase recognition motif may be omitted. For example, an acyl group, e.g., of formula

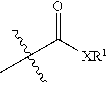

may replace the C-terminal amino acid of the sortase recognition motif. In some embodiments, the acyl group is

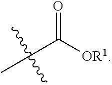

In certain embodiments, R¹ is substituted aliphatic. In certain embodiments, R¹ is unsubstituted aliphatic. In some embodiments, R¹ is substituted C₁₋₁₂ aliphatic. In some embodiments, R¹ is unsubstituted C₁₋₁₂ aliphatic. In some embodiments, R¹ is substituted C₁₋₆ aliphatic. In some embodiments, R¹ is unsubstituted C₁₋₆ aliphatic. In some embodiments, R¹ is C₁₋₃ aliphatic. In some embodiments, R¹ is butyl. In some embodiments, R¹ is n-butyl. In some embodiments, R¹ is isobutyl. In some embodiments, R¹ is propyl. In some embodiments, R¹ is n-propyl. In some embodiments, R¹ is isopropyl. In some embodiments, R¹ is ethyl. In some embodiments, R¹ is methyl. In certain embodiments, R¹ is substituted aryl. In certain embodiments, R¹ is unsubstituted aryl. In certain embodiments, R¹ is substituted phenyl. In certain embodiments, R^{1 is unsubstituted phenyl. In some embodiments, the acyl group is}

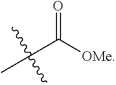 [SMILES: *C(=O)OC]

In some embodiments, the agent to be conjugated to the target protein comprises a protein. In some embodiments, the agent comprises a peptide. In some embodiments, the agent comprises a binding agent. In some embodiments, the agent comprises biotin. In some embodiments, the agent comprises streptavidin. In some embodiments, the agent comprises an antibody, an antibody chain, an antibody fragment, an antibody epitope, an antigen-binding antibody domain, a VHH domain, a single-domain antibody, a camelid antibody, a nanobody, or an adnectin. In some embodiments, the agent comprises a recombinant protein, a protein comprising one or more D-amino acids, a branched peptide, a therapeutic protein, an enzyme, a polypeptide subunit of a multisubunit protein, a transmembrane protein, a cell surface protein, a methylated peptide or protein, an acylated peptide or protein, a lipidated peptide or protein, a phosphorylated peptide or protein, or a glycosylated peptide or protein. In some embodiments, the agent is an amino acid sequence comprising at least 3 amino acids. In some embodiments, the agent comprises a fluorophore, a chromophore, or a fluorescent or phosphorescent moiety, or a radiolabel. In some embodiments, the agent comprises green fluorescent protein. In some embodiments, the agent comprises ubiquitin. In some embodiments, the agent comprises a small molecule. In some embodiments, the agent comprises a drug.

In certain embodiments, n (designating the number of amino acids in the N-terminal sortase recognition motif) is an integer from 0 to 50, inclusive. In certain embodiments, n is an integer from 0 to 20, inclusive. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6.

Any sortase that can carry out a transpeptidation reaction under conditions suitable for maintaining structural and functional integrity of the viral particle and the viral capsid protein to be modified can be used this invention. Examples of suitable sortases include, but are not limited to sortase A and sortase B, for example, from Staphylococcus aureus, or Streptococcus pyogenes. Additional sortases suitable for use in this invention will be apparent to those of skill in the art, including, but not limited to any of the 61 sortases described in Dramsi S, Trieu-Cuot P, Bierne H, Sorting sortases: a nomenclature proposal for the various sortases of Gram-positive bacteria. Res Microbiol. 156(3):289-97, 2005, the entire contents of which are incorporated herein by reference. Sortases belonging to any class of sortases, e.g., class A, class B, class C, and class D sortases, and sortases belonging to any sub-family of sortases (subfamily 1, subfamily 2, subfamily 3, subfamily 4 and sub-family 5) can be used in this invention.

Any amino acid sequence recognized by a sortase can be used the present invention. It will be understood by those of skill in the art, however, that in order for a certain sortase to carry out a transpeptidation reaction, the sortase recognition motif of the target protein to be modified and the sortase recognition motif the agent is conjugated to need to be recognized by that sortase. Numerous suitable sortase recognition motifs are provided herein, and additional suitable sortase recognition motifs will be apparent to the skilled artisan. Aside from naturally occurring sortase recognition motifs, some embodiments of this invention contemplate the use of non-naturally occurring sortase recognition motifs and sortases recognizing such motifs, for example, sortase motifs and sortases described in Piotukh et al., Directed evolution of sortase A mutants with altered substrate selectivity profiles. J Am Chem Soc. 2011 Nov. 9; 133(44):17536-9; and Chen I, Dorr B M, and Liu D R. A general strategy for the evolution of bond-forming enzymes using yeast display. Proc Natl Acad Sci USA. 2011 Jul. 12; 108(28):11399-404; the entire contents of each of which are incorporated herein by reference. In some embodiments, a recognition sequence, e.g., a sortase recognition sequence as provided herein further comprises one or more additional amino acids, e.g., at the N and/or C terminus. For example, one or more amino acids (e.g., up to 5 amino acids) having the identity of amino acids found immediately N-terminal to, or C-terminal to, a five amino acid recognition sequence in a naturally occurring sortase substrate may be incorporated. Such additional amino acids may provide context that improves the recognition of the recognition motif.

### Functionalization of M13 Phage Particles

The methods for functionalization of viral proteins via sortase-mediated transpeptidation provided herein can be used to modify surface proteins on any virus. As described in the Examples section herein, the method has been demonstrated to be capable to efficiently modify surface proteins of the bacteriophage M13. However, it will be apparent to those of skill in the art that the methods, reagents, and kits provided herein can be used to modify and functionalize surface proteins on other viruses as well.

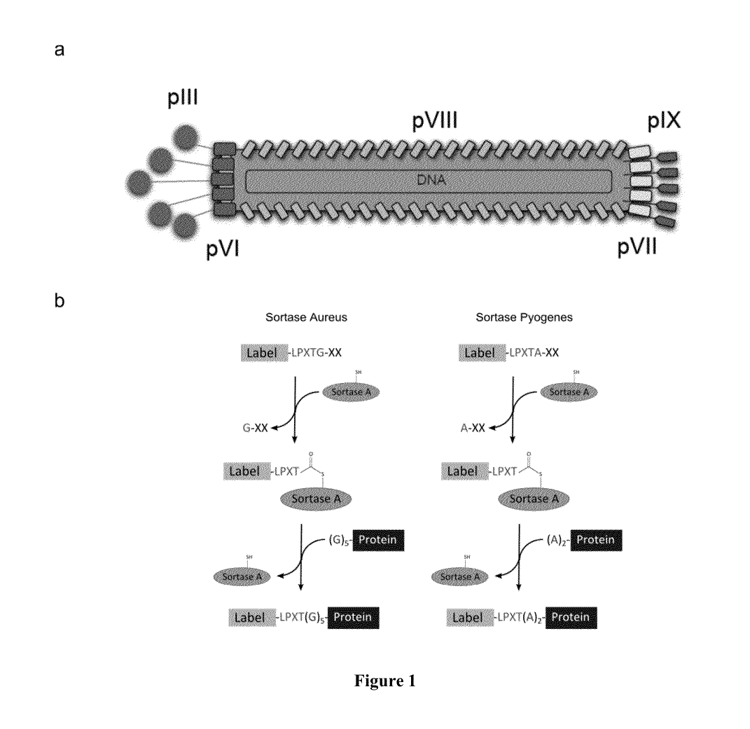

Wild type M13 bacteriophage has a cylindrical shape with a length of about 880 nm and a diameter of about 6 nm. It encapsulates a single-strand genome that encodes five different capsid proteins (FIG. 1A). The body of the phage is composed of 2700 copies of pVIII, the major capsid protein. At one end of the virus, there are ˜5 copies of both pIII and pVI proteins, and at the other end there are ˜5 copies of both pVII and pIX proteins¹.

The capsid proteins of M13 bacteriophage have been used to express combinatorial peptide libraries or protein variants (ranging from single domains to antibodies) to screen for target ligands in a process known as phage display². This technique has enabled not only identification of peptides with affinity for biological targets such as proteins, cells, and tissues³⁻⁶, but also allowed the identification of biomolecules that bind inorganics⁷⁻⁸. These molecules, when expressed on the M13 capsid proteins, can serve as scaffolds for nanowires, structures, and devices⁹⁻¹³. Functionalization of a virion capsid such as M13 is currently accomplished using chemical and/or genetic approaches¹⁴⁻¹⁵. However both strategies have limitations. Chemical conjugations are convenient and versatile, but they label motifs found on multiple M13 capsid proteins and oftentimes require non-physiological pH and reducing conditions that compromise the activity of the molecule that is being attached or of the moieties already displayed on other capsid proteins¹⁴.

Genetic engineering of phage allows the encoded protein/peptide to be displayed precisely^{13, 16}, but it has intrinsic restrictions. Two classes of vectors are available for genetic phage display: phagemid and phage. A phagemid allows expression of large fusions with any of the five M13 phage capsid proteins, but these fusions are incorporated at low efficiency¹⁷⁻²¹. In a phage vector, the M13 bacteriophage genome is modified directly. As a result, every copy of the recombinant capsid protein incorporated into the virus displays the modified protein. However, this strategy does not support display of large moieties²²⁻²⁴. pVIII allows the display of a larger number of recombinant molecules per phage particle, but it also has the strictest size limitation in phage vector display. pVIII peptide libraries are mostly limited to sizes of up to 10 amino acids, as phage with longer insertions rarely assemble²⁵⁻²⁶. Insertions of 6-20 amino acids onto pVIII are possible using phagemid, but their display is inefficient with less than 25% of the copies of pVIII containing the desired fusion product²⁰. Incorporation of proteins is even less efficient on pVIII: a 23 kDa protein is displayed, on average, on less than a single copy of the pVIII fusion per phage particle using a phagemid vector¹⁸. Phage display methods on the pVIII have been able to increase the binding affinity of phage displaying a moiety²³, but the displayed copy number of the moiety has not been determined. Large moieties of at least 23 kDa have been genetically fused to all four minor capsid proteins using a phagemid vector^{22, 27-28}, but only pIII has been extensively used in the phage vector system²⁹. However, viability of the resultant phage fusions does not guarantee that the recombinant peptide/protein of interest displays its native structure and/or maintains its wild type function. Both the environment where phage assembles and the phage coat protein to which the protein of interest is fused may interfere with proper folding³⁰. This is particularly critical for enzymes and antibodies as they might not be functional when incorporated into the phage structure.

The technology provided by this disclosure expands the versatility of M13 as a display platform, by employing a strategy based on sortase-mediated chemo-enzymatic reactions to covalently attach a variety of moieties to the N-terminus of pIII, pVIII, and pIX. The technology provided herein allows for the conjugation of functional moieties and molecules at a high efficiency, as illustrated by a comparison to published labeling data described in more detail in the Examples section. For example, as described in more detail in the Examples section, the instantly described sortase-based functionalization technology represents a significant improvement over current methodologies in the copy number of displayed peptides and proteins, particularly on pVIII.

Sortase A enzymes allow modification of proteins by enzymatic ligation with a wide range of molecules, moieties, and functional groups (including biotin, fluorophores, and other proteins) at the C-terminus, N-terminus, or at both termini of the protein of interest³¹⁻³⁵ (see, e.g., Ploegh et al., International PCT Patent Application, PCT/US2010/000274, filed Feb. 1, 2010, published as WO/2010/087994 on Aug. 5, 2010, and Ploegh et al., International Patent Application PCT/US2011/033303, filed Apr. 20, 2011, published as WO/2011/133704 on Oct. 27, 2011, the entire contents of which are incorporated herein by reference). Different sortase enzymes are known to those of skill in the art, and any sortase carrying out a transpeptidation reaction can be used in the context of the instant disclosure. For example, the widely used sortase A from Staphylococcus aureus (SrtAₐᵤᵣₑᵤₛ) recognizes substrates that contain an LPXTG (SEQ ID NO: 78) sequence³⁶⁻³⁸, whereas sortase A from Streptococcus pyogenes (SrtA_{pyogenes}) recognizes substrates with an LPXTA (SEQ ID NO: 91) motif^{33,39}. The sortase enzymes cleave between the threonine and glycine or alanine residue, respectively, to yield a covalent acyl-enzyme intermediate that is resolved by nucleophilic attack of a suitably exposed amine, namely oligoglycine or oligoalanine-containing peptides³⁹ in the case of SrtAₐᵤᵣₑᵤₛ or SrtA_{pyogenes}, respectively (FIG. 1B). Some aspects of this invention provide methods and protocols using a plurality of orthogonal sortase A enzymes, e.g., SrtAₐᵤᵣₑᵤₛ and SrtA_{pyogenes}, to site-specifically conjugate two different moieties onto two different capsid proteins (e.g., pIII and pVIII) in a single phage particle.

The sortase labeling methods provided herein have several advantages over genetic and chemical methods. First, the sortase transpeptidation reaction is site-specific. This is advantageous, as it allows one to specifically target sortase activity towards a genetically engineered target protein. For example, in the case of sortagging of an M13 capsid protein, as none of the M13 coat proteins naturally display a sortase recognition motif required to participate in sortase-mediated reactions, a capsid protein engineered to comprise such a motif will be specifically targeted by a sortase, while the non-engineered proteins will not participate in the sortase reaction. Second, sortase recognition motifs are small and, therefore, can be easily inserted into the host genome, e.g., the M13 phage genome, thus maximizing the number of potential attachment sites. Third, a protein to be conjugated to a cell surface or particle surface protein by means of sortase, e.g., a protein to be displayed on a phage particle, can be properly folded separate from the conjugation reaction, and, as the case may be, separate from the assembly of phage particles. The site-specific nature of the reaction fixes the orientation of the displayed protein. Fourth, the reactions are performed under physiological conditions. Fifth, sortase reactions afford attachment of a wide range of molecules, including those that cannot be genetically encoded such as fluorophores and biotin.

Some aspects of this description provide reagents and methods to build phage structures that have new material and biological applications. Some non-limiting examples are described in detail: the creation of a new lampbrush structure by fusing different phage particles through pIII/pVIII, a fluorescently labeled phage containing a cell-targeting moiety to stain and to sort cells by FACS, and the formation of multiphage particles of a specific, predetermined structure via hybridization-mediated linkage of DNA oligonucleotides conjugated to pIII/pVIII of phage particles. It will be apparent to the skilled artisan that the described examples are illustrative and non-limiting, as various additional applications of the technology described herein will be apparent to the skilled artisan.

In some embodiments, the ability to fluorescently stain cells can be used in the panning of phage display libraries against specific cells. Phage particles functionalized with fluorescent moieties or proteins allow for more sensitive detection of binding events and/or for decreasing the number of panning rounds needed for identifying a biomolecule of interest in phage display screens.

The ability to generate structures using functionalized phage as building blocks can be used to produce complex hybrid material structures. For example, in some embodiments, functionalized phage particles can be created that can bind to and nucleate different materials, including other phage particles, organic materials, and inorganic materials. In some embodiments, hybrid structures of inorganic matter and phage particles can be generated.

Some aspects of this invention provide methods for associating viral particles, for example, M13 phage particles, with viral particles of the same type (e.g., with other M13 phage particles), with viral particles of a different type (e.g., with phage particles of a different strain), or with cells or other entities (e.g., with target cells, e.g., bacterial cells not typically bound or infected by wild-type M13 phage, or with non-target cells, e.g. yeast, insect, or mammalian cells, or with organic particles, e.g., nanoparticles).

Typically, a method for associating viral particles of the same type comprises conjugating a first target protein on the surface of the viral particle with a first binding agent via sortase-mediated transpeptidation; conjugating a second target protein on the surface of the viral particle with a second binding agent, wherein the second binding agent binds the first binding agent; and incubating a plurality of viral particles comprising the first and the second binding agent under conditions suitable for the first and the second binding agent of different viral particles to bind each other. In some embodiments, the first binding agent is a ligand-binding agent, for example, a receptor, or a receptor fragment, and the second binding agent comprises the ligand bound by the ligand-binding agent. For example, in some embodiments, the first binding agent is biotin, and the second binding agent is streptavidin. In some embodiments, the first binding agent comprises an antibody or an antigen-binding antibody fragment, and the second binding agent comprises the antigen bound by the antibody or antibody fragment. In some embodiments, an M13 capsid protein is sortagged with a first binding agent, e.g., pIII with biotin or a first oligonucleotide, and a second M13 capsid protein is sortagged with a second binding agent binding the first binding agent, e.g., pVIII with streptavidin or a second oligonucleotide. As described in more detail elsewhere herein, the M13 particles functionalized in this manner associate when incubated under suitable conditions, e.g., under suitable conditions for biotin and streptavidin to bind or under suitable conditions for the first and second oligonucleotide to become associated with each other (e.g., via hybridization to a third oligonucleotide), and can form complex, branched structures not observed in non-functionalized phage particles.

A method for associating viral particles of one type to viral particles of a different type typically comprises conjugating a target protein on the surface of a first viral particle with a first binding agent via sortase-mediated transpeptidation reaction; conjugating a target protein on the surface of a second viral particle with a second binding agent, wherein the second binding agent binds the first binding agent directly or can otherwise become associated with the first binding agent (e.g., by binding a molecule bound by the first binding agent); and contacting and incubating a plurality of viral particles comprising the first binding agent with a plurality of viral particles comprising the second binding agent under conditions suitable for the first and the second binding agent of different viral particles to bind each other. In some embodiments, the first binding agent is a ligand-binding agent, for example, a receptor, or a receptor fragment, or an adhesion molecule, and the second binding agent comprises the ligand bound by the ligand-binding agent. For example, in some embodiments, the first binding agent is biotin and the second binding agent is streptavidin. In some embodiments, the first binding agent comprises an antibody or an antigen-binding antibody fragment, and the second binding agent comprises the antigen bound by the antibody or antibody fragment. In some embodiments, an M13 capsid protein of a first M13 particle is sortagged with a first binding agent, e.g., pIII with biotin, and a second M13 capsid protein of a second M13 particle is sortagged with a second binding agent binding the first binding agent, e.g., pVIII with streptavidin. In other embodiments, the same capsid protein is sortagged with a first binding agent on a first M13 particle and with a second binding agent on a second M13 particle, e.g., pVIII is sortagged with biotin on a first M13 particle and with streptavidin on a second M13 particle. The M13 particles functionalized in this manner are then incubated under conditions suitable for them to associate, resulting in a branched structure of associated, differently sortagged M13 particles.

Viral particles can be functionalized with any suitable binding agent, for example, with a binding agent binding an antigen or ligand on the surface of a cell, e.g., a bacterial cell, a yeast cell, an insect cell, a vertebrate cell, or a mammalian cell. Incubation of the functionalized viral particle with the cell results in binding of the functionalized viral particle to the cell. In some embodiments, the binding agent is biotin/streptavidin. Other suitable binding agents include, without limitation, complementary DNA strands, ligands of receptors expressed on the surface of the target cells, and leucine zippers. In some embodiments, direct attachment of phage to a cell or other biological structure is effected by placing a sortase substrate on the surface of the phage, and a compatible sortase substrate on the surface of the cell or biological structure and then effecting a sortase-mediated transpeptidation reaction between the two. Association of viral particles and cells can be achieved if a plurality of particles is contacted with a plurality of cells under suitable conditions. The association of viral particles with other viral particles of a different type, or with cells, e.g., with cells that are not naturally bound or infected by the viral particles allows for the generation of novel hybrid structures and materials the characteristics of which will be determined by the structure of the associated entities, and by the agents and target proteins used for functionalization of the viral particles.

### Functionalized Viral Particles

Some aspects of this invention provide functionalized viral particles, in which at least one viral capsid protein has been sortagged according to methods, or using reagents or strategies provided herein. In some embodiments, the functionalized virus comprises a target protein, for example, a viral capsid protein, that is conjugated to an agent via a sortase recognition motif as described herein. In some embodiments, the agent is conjugated to the target protein via a linker. In some embodiments, the linker is a peptide linker, e.g., a linker comprising a sequence of amino acids. In some embodiments, the linker is a cleavable linker, for example, a linker comprising a protease cleavage site, or a photocleavable linker. Cleavable linkers including, but not limited to linkers comprising protease cleavage sites and photocleavable linkers, are well known to those of skill in the art, and the invention is not limited in this respect. In some embodiments, the agent has been conjugated to the target protein by a sortase-mediated transpeptidation reaction, e.g., by a method provided herein. Typically, a sortase-mediated transpeptidation reaction leaves a “scar” in the generated protein, which comprises the C-terminal sortase recognition motif (e.g., LPXT, or any other C-terminal sortase recognition motif described herein) and, in some embodiments, a plurality of N-terminal amino acids comprised in the respective N-terminal sortase recognition motif, e.g., (G)ₙ or (A)ₙ, wherein n is an integer equal to or greater than 2. The sortase recognition motif in the product of the transpeptidation reaction is typically a sequence created by the sortase reaction, e.g., by a SrtAₐᵤᵣₑᵤₛ mediated transpeptidation reaction or by a SrtA_{pyogenes} transpeptidation reaction.

In some embodiments, the agent conjugated to the capsid protein is a protein, a detectable label, a binding agent, a click-chemistry handle, a small molecule, or any other agent described herein. In some embodiments, the virus comprises a plurality of different target proteins conjugated to an agent (e.g., different types of target proteins to different agents) via a sortase recognition motif. In some embodiments, different target proteins of the virus are conjugated to different agents, for example, a binding agent and a detectable label; two different detectable labels; a first binding agent, a second binding agent, and a detectable label, and so on. In some embodiments, the different target proteins are conjugated to the respective agents via sortase recognition motifs of orthogonal sortases. For example, in some embodiments, a virus is provided comprising a first target protein conjugated to a first agent via a SrtAₐᵤᵣₑᵤₛ recognition motif, and a second target protein conjugated to a second agent via a SrtA_{pyogenes} recognition motif.

In some embodiments, a functionalized M13 bacteriophage is provided that comprises a pIII conjugated to an agent via a sortase recognition motif. In some embodiments, a functionalized M13 bacteriophage is provided that comprises a pVIII conjugated to an agent via a sortase recognition motif. In some embodiments, a functionalized M13 bacteriophage is provided that comprises a pIX conjugated to an agent via a sortase recognition motif. In some embodiments, the agent is an agent as described herein, for example, a binding agent or a detectable label. In some embodiments, a functionalized M13 bacteriophage is provided that comprises a pIII conjugated to a first agent, and a pVIII conjugated to a second, different agent. In some embodiments, a functionalized M13 bacteriophage is provided that comprises a pIII conjugated to a first agent, and a pIX conjugated to a second, different agent. In some embodiments, a functionalized M13 bacteriophage is provided that comprises a pVIII conjugated to a first agent, and a pIX conjugated to a second, different agent. In some embodiments, the first agent is a binding agent (e.g., biotin). In some embodiments, the second agent is a binding agent that binds the first binding agent (e.g., streptavidin). Additional suitable agents include, but are not limited to, click chemistry handles, SNAP-, Clip-, ACP-, and MCP-tags, complementary DNA strands, leucine zippers, GFP, and toxins, e.g., bacterial and plant toxins In some embodiments, three different target proteins are conjugated to three different agents, four different agents to four different target proteins, and so on. The invention is not limited in this respect.

The virus may be any virus suitable for sortase-mediated functionalization as described herein, including, but not limited to, a dsDNA virus comprising a double-stranded DNA genome, an ssDNA virus comprising a single-stranded DNA genome, a dsRNA virus comprising a double-stranded RNA genome, a (+)ssRNA virus comprising a single stranded (+)sense strand RNA genome, a (−)ssRNA virus comprising a single stranded (−)sense RNA, an ssRNA-RT virus comprising a single-stranded (+)sense RNA with a DNA intermediate genome in its life-cycle that is generated by reverse transcription of the RNA genome, or a dsDNA-RT virus. Exemplary functionalized viruses include, e.g., Retroviridae (e.g., lentiviruses such as human immunodeficiency viruses, such as HIV-I); Caliciviridae (e.g. strains that cause gastroenteritis); Togaviridae (e.g. equine encephalitis viruses, rubella viruses); Flaviridae (e.g. dengue viruses, encephalitis viruses, yellow fever viruses, hepatitis C virus); Coronaviridae (e.g. coronaviruses); Rhabdoviridae (e.g. vesicular stomatitis viruses, rabies viruses); Filoviridae (e.g. Ebola viruses); Paramyxoviridae (e.g. parainfluenza viruses, mumps virus, measles virus, respiratory syncytial virus); Orthomyxoviridae (e.g. influenza viruses); Bunyaviridae (e.g. Hantaan viruses, bunga viruses, phleboviruses and Nairo viruses); Arenaviridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae; Herpesviridae (herpes simplex virus (HSV) 1 and 2, varicella zoster virus, cytomegalovirus (CMV), EBV, KSV); Poxyiridae (variola viruses, vaccinia viruses, pox viruses); and Picornaviridae (e.g. polio viruses, hepatitis A virus; enteroviruses, human coxsackie viruses, rhinoviruses, echoviruses). In some embodiments, the functionalized virus provided is a DNA virus. In some embodiments, the functionalized virus is a phage, or bacteriophage. In some embodiments, the functionalized virus is a filamentous phage. In some embodiments, the functionalized virus is an M13 bacteriophage. In some embodiments, the functionalized virus provided is a bacteriophage, for example, a bacteriophage belonging to the family of Myoviridae (e.g., T4 phage), Siphoviridae (e.g., λ phage, Bacteriophage T5), Podoviridae (e.g., T7 phage), Ligamenvirales, Lipothrixviridae, Rudiviridae, Ampullaviridae, Bacilloviridae, Bicaudaviridae, Clavaviridae, Corticoviridae, Cystoviridae, Fuselloviridae, Globuloviridae, Guttavirus, Inoviridae, Leviviridae (e.g., MS2, Qβ), Microviridae (e.g., ΦX174), Plasmaviridae, or Tectiviridae. Exemplary functionalized bacteriophages provided herein include, without limitation, Lambda phage (λ phage, lysogen), T2 phage, T4 phage, T7 phage, T12 phage, R17 phage, M13 phage, MS2 phage, G4 phage, P1 phage, Enterobacteria phage P2, P4 phage, ΦX174 phage, N4 phage, Φ6 phage, and Φ29 phage. Further, any virus that may be functionalized using the methods, reagents, and/or kits provided herein is within the scope of the present invention, including, but not limited to, those viruses described on pages 129-653 of Stephen T. Abedon, The Bacteriophages, Oxford University Press, USA; 2^{nd} edition, Dec. 15, 2005, ISBN: 0195148509; the entire contents of which are incorporated herein by reference.

Some aspects of this invention provide viruses that comprise an engineered capsid protein comprising a sortase recognition motif, for example, a C-terminal or N-terminal sortase recognition motif described herein. Such engineered viruses can readily be functionalized according to methods described herein without the need for further engineering of the virus, for example, using recombinant methods. For example, in some embodiments, a phage is provided that comprises a capsid protein that does not naturally comprise a sortase recognition motif at a terminus that is accessible on the surface of the phage. In some embodiments, the phage is an M13 phage, comprising an engineered capsid protein, for example, a pIII, pVIII, or pIX protein comprising a recombinant poly-glycine or poly-alanine sequence (e.g., (G)ₙ or (A)ₙ, wherein n is equal to or greater than 2 at its N-terminus.

Some aspects of this invention provide nucleic acids encoding an engineered capsid protein comprising a sortase recognition motif. Such nucleic acids can be used to generate virus particles comprising the engineered capsid proteins, which can then be functionalized according to the methods described herein. In some embodiments, an isolated nucleic acid is provided that encodes a viral capsid protein comprising an N-terminal or a C-terminal sortase recognition motif. In some embodiments, the nucleic acid is a recombinant nucleic acid. In some embodiments, the sortase recognition motif is inserted into a wild-type nucleic acid sequence encoding the capsid protein. In some embodiments, the nucleic acid is comprised in an expression vector. Such vectors are also provided by aspects of this invention. Such expression vectors typically comprise the encoding nucleic acid and additional nucleic acid elements mediating the expression and/or replication of the nucleic acid in a host cell, for example, a bacterial host cell in the case of bacteriophages. In some embodiments, the expression construct also comprises nucleic acid sequences encoding one or more additional capsid proteins of the virus. In some embodiments, the expression construct encodes at least two engineered capsid proteins, each comprising a sortase recognition motif. In some embodiments, the sortase recognition motifs comprised in the at least two engineered capsid proteins are recognized by orthogonal sortases. In some embodiments, proteins encoded by the nucleic acids and expression constructs described herein are provided.

### Kits

Some aspects of this invention provide kits useful for the expression of viral capsid proteins comprising a sortase recognition motif, and for the generation of viral particles that can be functionalized via a sortagging technique described herein. In some embodiments, such a kit comprises a recombinant nucleic acid encoding a viral capsid protein comprising a sortase recognition motif. In some embodiments, the kit further comprises a nucleic acid encoding additional viral genes. In some embodiments, the additional viral genes may comprise at least one additional capsid protein comprising a sortase recognition motif. In some embodiments, the kit comprises nucleic acid sequences encoding two or more capsid proteins comprising different sortase recognition motifs. In some embodiments, the different sortase recognition motifs are recognized by orthogonal sortases, for example, one by SrtAₐᵤᵣₑᵤₛ and another by SrtA_{pyogenes}. In some embodiments, the kit comprises one or more nucleic acid molecules that together provide all viral genes necessary to generate a viral particle. For example, in some embodiments, the kit provides a nucleic acid sequence encoding M13 pIII comprising a sortase recognition sequence (e.g., poly-glycine) at its N-terminus, and also one or more nucleic acid sequences encoding the M13 genome except wild-type pIII. In some embodiments, the kit provides a nucleic acid sequence encoding M13 pIII comprising a sortase recognition sequence (e.g., poly-glycine) at its N-terminus, a nucleic acid sequence encoding M13 pVIII comprising a sortase recognition sequence (e.g., poly-alanine) at its N-terminus, and one or more nucleic acid sequences encoding the M13 genome except wild-type pIII and pVIII. In some embodiments, the kit provides a nucleic acid sequence encoding M13 pVIII comprising a sortase recognition sequence (e.g., poly-glycine) at its N-terminus, a nucleic acid sequence encoding M13 pIX comprising a sortase recognition sequence (e.g., poly-alanine) at its N-terminus, and one or more nucleic acid sequences encoding the M13 genome except wild-type pVIII and pIX.

Some kits provided herein comprise the nucleic acids described herein as part of one or more expression constructs. Expression constructs may be in the form of a vector, e.g., a plasmid or phagemid, which can readily be introduced into a host cell, e.g., a bacterial cell that can be infected by a bacteriophage, to generate recombinant viral particles, e.g., M13 particles comprising an M13 pIII protein that contains a sortase recognition motif. Recombinant phage generated from such kits can then be functionalized by a sortagging method described herein.

In some embodiments, the kit further comprises a sortase. Typically, the sortase comprised in the kit recognizes a sortase recognition motif encoded by a nucleic acid comprised in the kit. In some embodiments, the sortase is provided in a storage solution and under conditions preserving the structural integrity and/or the activity of the sortase. In some embodiments, where two or more orthogonal sortase recognition motifs are encoded by the nucleic acid(s) comprised in the kit, a plurality of sortases is provided, each recognizing a different sortase recognition motif encoded by the nucleic acid(s). In some embodiments, the kit comprises SrtAₐᵤᵣₑᵤₛ and/or SrtA_{pyogenes}.

In some embodiments, the kit further comprises a sortase substrate. In some embodiments, the sortase substrate comprises a sortase recognition motif conjugated to an agent. For example, the kit may comprise a sortase substrate comprising a sortase recognition motif that is compatible with a sortase recognition motif encoded by a nucleic acid in the kit in that both motifs can partake in a sortase-mediated transpeptidation reaction catalyzed by the same sortase. For example, if the kit comprises a nucleic acid encoding a capsid protein comprising a SrtAₐᵤᵣₑᵤₛ N-terminal recognition sequence, the kit may also comprise SrtAₐᵤᵣₑᵤₛ and a SrtAₐᵤᵣₑᵤₛ substrate conjugated to an agent, wherein the sortase substrate will comprise the C-terminal sortase recognition motif. In some embodiments, the kit further comprises a buffer or reagent useful for carrying out a sortase-mediated transpeptidation reaction, for example, a buffer or reagent described in the Examples section.

The following working examples are intended to describe exemplary reductions to practice of the methods, reagents, and compositions provided herein and do not limited the scope of the invention.

### EXAMPLES

### Example 1

### Sortase-Mediated Modification of M13 Phage Surface Proteins

### Experimental Procedures

Generation of the M13 Phage Constructs.

The oligonucleotides used to design the different phage constructs are compiled in Table 3. The G₅-pIII phage (SEQ ID NO: 77) was engineered by inserting the G5pIIIC and G5pIIINC (SEQ ID NO: 77) annealed oligonucleotides into the M13KE vector (New England Biolabs), previously digested with EagI and Acc65I restriction enzymes. To construct the A₂G₄-pVIII phage, the M13SK vector⁴⁰ was digested with PstI and BamHI restriction enzymes and the A2G4pVIIIC (SEQ ID NO: 9) and A2G4pVIIINC (SEQ ID NO: 9) annealed oligonucleotides were inserted. To engineer the G₅HA-pIX construct (SEQ ID NO: 77), the 983 vector was used. This vector was created by refactoring the M13SK vector so the pIX and pVII genes are not overlapping. Upon digestion of this vector with SfiI, the annealed G5HApIXC and G5HApIXNC (SEQ ID NO: 77) oligonucleotides were inserted. The G₅-pIII-A₂-pVIII (SEQ ID NO: 77) phage construct was created using a modified M13SK vector⁴⁰, which has a DSPHTELP (SEQ ID NO: 116) sequence on pVIII and a biotin acceptor peptide (GLQDIFEAQKIEWHE (SEQ ID NO: 117)) on pIII. Five N-terminal glycines were added to pIII following the above strategy described for G₅-pIII phage (SEQ ID NO: 77). The resultant vector was then modified at the N-terminus of pVIII using the QuikChange II site-directed mutagenesis kit (Stratagene) and the pVIIIAADSPH oligonucleotide pair. All the generated phage vectors were transformed into the XL-1 Blue bacterial strain, plated in agar top on LB agar plates containing 1 mM IPTG, 40 μg/mL X-Gal, and 30 μg/mL tetracycline. Plaques were selected and DNA was isolated and sequenced to check for the insertion.

TABLE 3
Oligonucleotides for phage engineering

| Name | Sequence (5′-3′) |
|---|---|
| G5pIIIC | GTACCTTTCTATTCTCACTCTGGTGGAGGCGGTGGATC (SEQ ID NO: 1) |
| G5pIIIINC | GGCCGATCCACCGCCTCCACCAGAGTGAGAATAGAAAG (SEQ ID NO: 2) |
| A2G4pVIIIC | GCTGGCGGGGGAGGG (SEQ ID NO: 3) |
| A2G4pVIIINC | GATCCCCTCCCCCGCCAGCTGCA (SEQ ID NO: 4) |
| G5HApIXC | CGGCCATGGCGGGCGGAGGTGGAGGCTACCCATACGATGTTCCAGATT |
| | ACGCTCAGGG (SEQ ID NO: 5) |
| G5HApIXNC | TGAGCGTAATCTGGAACATCGTATGGGTAGCCTCCACCTCCGCCCGCC |
| | ATGGCCGGCT (SEQ ID NO: 6) |
| AADSPH-pVIII-Top | GTTCCGATGCTGTCTTTCGCTGCTGCAGATTCGCCGCATACTGAG (SEQ |
| | ID NO: 7) |
| AADSPH-pVIII- | CTCAGTATGCGGCGAATCTGCAGCAGCGAAAGACAGCATCGGAAC |
| Bottom | (SEQ ID NO: 8) |

For phage amplification, the E. coli strain ER2738 (New England Biolabs) in LB media supplemented with 30 μg/mL tetracycline, was infected with phage for at least 12 hrs at 37° C. The cultures were centrifuged at 12000 g for 20 min and the phage was precipitated from the supernatant at 4° C. with the addition of ⅕ of the supernatant volume of 20% PEG8000/2.5M NaCl solution. Upon centrifugation at 13500 g for 20 min, the pellet was resuspended in 25 mM Tris, 150 mM NaCl, pH 7.0-7.4 (TBS). For further purification, this resuspension was subjected to two rounds of centrifugation/precipitation. The final phage concentration averaged between 10¹³-10¹⁴ plaque forming units (pfu) per mL as determined by UV-vis spectrometry⁴¹.

Sortase-Mediated Reactions.

SrtA_{pyogenes} and SrtAₐᵤᵣₑᵤₛ were expressed and purified as described^{33, 42}. Sortase reactions were performed as indicated in the figures. A typical sortase reaction with SrtAₐᵤᵣₑᵤₛ included 200 nM phage, 50 μM SrtAₐᵤᵣₑᵤₛ, and 50 μM substrate for small peptides or 20 μM for proteins. The reactions were incubated for 3 hrs at 37° C. (for small peptides) or at room temperature (for proteins) in TBS with 10 mM CaCl₂. SrtA_{pyogenes}-mediated reactions included 8 nM phage, 50 μM SrtA_{pyogenes}, and 20 μM substrate, incubated for 3 hr at 37° C. in TBS. Where indicated, phage was purified by PEG 8000/NaCl precipitation after diluting the reactions with TBS such that the substrate concentration was below 600 nM.

For the flow cytometry experiments, the G₅-pIII-A₂-pVIII (SEQ ID NO: 77) phage construct was labeled with K(TAMRA)-LPETAA (SEQ ID NO: 12) on pVIII. The resultant labeled phage was purified by PEG8000/NaCl precipitation, resuspended in TBS, and split into three parts. One part remained unlabeled, and the other two were labeled with either VHH7.LPETG (SEQ ID NO: 10) or anti-GFP.LPETG (SEQ ID NO: 10) on pIII. As assessed by the anti-pIII antibody, a yield of 2.5 antibody molecules per virion was achieved in both cases.

The yield of the sortase-mediated biotinylation reactions was determined using biotinylated GFP as a standard. This was prepared labeling GFP—comprising a LPETG (SEQ ID NO: 10) at its C-terminus—with a biotin group using SrtAₐᵤᵣₑᵤₛ (GFP.LPETGGGK(biotin))⁴² (SEQ ID NO: 281). Known amounts of the purified GFP.LPETGGGK(biotin) standard (SEQ ID NO: 281) and varying volumes of the phage labeling reactions were loaded onto the same SDS-PAGE gel and analyzed by immunoblot using streptavidin-HRP (GE Healthcare). The signal obtained in the phage labeling reactions was compared with the signal derived from the GFP.LPETGGGK(biotin) (SEQ ID NO: 281) calibration curve allowing us to infer the amount of phage protein labeled in the reaction. To calculate the labeling efficiency, the amount of labeled protein was divided by the amount of total phage protein loaded into the gel. The phage concentration was determined by UV-vis spectrometry and it was assumed that there were 2700 copies of pVIII, 5 copies of pIII, and 5 copies of pIX per phage particle.

To determine the yield of GFP-pVIII phage labeling, unincorporated GFP and sortase was removed from phage by PEG8000/NaCl precipitation. Varying volumes of GFP-pVIII phage and known amounts of GFP were loaded onto the same SDS-PAGE gel and analyzed by immunoblot using an anti-GFP-HRP antibody (Santa Cruz Biotechnology). The signal of the GFP-pVIII fusion protein was compared to the signal of the GFP calibration curve as described for the biotinylation reactions. For GFP-pIII and GFP-pIX labeling, the signal of the fusion protein was compared to the input amount of pIII or pIX as detected by anti-pIII (New England Biolabs) or anti-HA (Roche) antibodies, respectively. For GFP-pIII, the input signal consisted of only intact pIII molecules and lower molecular weight anti-pIII reactive proteins were not included. These proteins can be attributed to proteolyzed pIII⁴³. Because the anti-pIII antibody recognizes the C-terminus of the protein, these fragments cannot be labeled using SrtAₐᵤᵣₑᵤₛ. In all cases the blots were scanned and densitometric analysis was performed using the ImageJ program (National Institutes of Health). The labeling yield was averaged over three independent reactions with three aliquots from each reaction analyzed. The standard deviation of the reactions was calculated from the averages of the three independent reactions.

Dynamic Light Scattering (DLS).

DLS measurements were obtained with a Beckman Delsa-Nano C Particle Analyzer (Beckman Coulter Inc). Phage mixtures were diluted to ˜10¹¹ pfu/mL in 1 mL of water and loaded into a cuvette. Samples from each experiment were measured in triplicate and the results were averaged by cumulant analysis. Autocorrelation functions were used as a direct comparison of aggregation because aggregates have a slower Brownian motion causing the signal correlation to be delayed to longer relaxation times.

Atomic Force Microscopy (AFM).

Phage preparations were diluted to a concentration of ˜10¹¹ pfu/mL, and 100 μL of this mixture were deposited on a freshly cleaved mica disc. AFM images were taken on a Nanoscope IV (Digital Instruments) in air using tapping mode. The tips had spring constants of 20-100N/m driven near their resonant frequency of 200-400 kHz (MikroMasch). Scan rates were approximately 1 Hz. Images were leveled using a first-order plane fit to remove sample tilt.

Flow Cytometry Analysis.

C57BL/6 mice were purchased from Jackson Labs. Animals were housed at the Whitehead Institute for Biomedical Research and were maintained according to guidelines approved by the Massachusetts Institute of Technology (MIT) Committee on Animal Care. Lymph nodes were isolated from 6-8 week old C57BL/6 mice and crushed through a 40 μM cell strainer. Cells were washed once with PBS, resuspended at 2×10⁷ cells per mL, aliquoted at ˜1×10⁶ cells per sample, and incubated with staining agents in 5% milk in PBS for 1 hr at room temperature. 10¹¹ VHH7 molecules and 10¹¹ anti-GFP molecules either directly conjugated to TAMRA using SrtAₐᵤᵣₑᵤₛ, or covalently attached to phage (5×10¹⁰ phage particles of VHH7-G₅-pIII-TAMRA-A₂-pVIII (SEQ ID NO: 77) or anti-GFP-G₅-pIII-TAMRA-A₂-pVIII (SEQ ID NO: 77), see Sortase-mediated reactions section) were incubated with the cells. The same amount of non-targeted fluorescent phage particles (i.e., G₅-pIII-TAMRA-A₂-pVIII) (SEQ ID NO: 77) was used as a negative control. B cells were stained with Pacific Blue anti-mouse B220 (BD Pharmingen, clone RA3-6B2). Upon staining, the cells were centrifuged at 170 g for 5 min, washed with PBS three times, and resuspended in 500 μL of PBS. Flow cytometry was performed using a FACSAria (BD). 100,000 events were collected for each sample.

Estimating Nearest Neighbor Packing of GFP on Phage Surface.

Using the crystal structure of the pVIII capsid protein (1IFJ, see Marvin, D. A., Hale, R. D., Nave, C., and Helmer-Citterich, M. (1994) Molecular models and structural comparisons of native and mutant class I filamentous bacteriophages Ff (fd, fl, M13), Ifl and IKe. J. Mol. Biol. 235, 260-86.), a model viral capsid was constructed with fivefold symmetry serving as a model of the phage surface. A crystal structure of GFP (1GFL, see, Yang, F., Moss, L. G., and Phillips, G. N., Jr. (1996) The molecular structure of green fluorescent protein. Nat. Biotechnol. 14, 1246-51) was oriented such that its C-terminus was adjacent to the N-terminus of pVIII. By analyzing this image, it was determined that one GFP molecule blocked the N-termini of the six pVIII proteins surrounding the GFP-pVIII fusion meaning at most one out of seven pVIII proteins can be labeled with a GFP. From this, it was calculated that a single virion with 2700 pVIII proteins would have at most 385 GFP molecules. The visualizations were performed using WinCoot (see Emsley, P., Lohkamp, B., Scott, W. G., and Cowtan, K. (2010) Features and development of Coot. Acta Crystallogr. D Biol. Crystallogr. 66, 486-501). All references referred to in the above paragraph are incorporated herein by reference in their entirety.

Miscellaneous.

Expression and purification of GFP.LPETG.His₆ (SEQ ID NO: 287) and GFP.LPETA.His₆ (SEQ ID NO: 283), were performed as described³³. Identification, characterization, expression, and purification of VHH7.LPETG.His₆ (SEQ ID NO: 287) will be published elsewhere. Streptavidin was cloned as a streptavidin.LPETG.HAtag.His₆ (SEQ ID NO: 10 and 288) fusion protein using the template Addgene 20860⁴⁴, and expressed as a soluble tetrameric streptavidin⁴⁵. Purification was performed following the same protocol used for GFP³³. Sortase reactions were analyzed on 4-12% Bis-Tris SDS-PAGE gels with MES running buffer except for FIG. 10 which was analyzed on a 12% Laemmli SDS-PAGE gel.

The K(biotin)-LPETGG (SEQ ID NO: 13), K(biotin)-LPETAA (SEQ ID NO: 12), K(TAMRA)-LPETAA (SEQ ID NO: 12), and GGGK(biotin) (SEQ ID NO: 127) peptides were obtained from the Swanson Biotechnology Center. For mass spectrometry, the protein bands of interest were excised, subjected to protease digestion, and analyzed by electrospray ionization tandem mass spectrometry (MS/MS). Fluorescent gel images were obtained using a variable mode imager (Typhoon 9200; GE Healthcare).

### Results

N-Terminal Labeling of pIII Using SrtAₐᵤᵣₑᵤₛ.

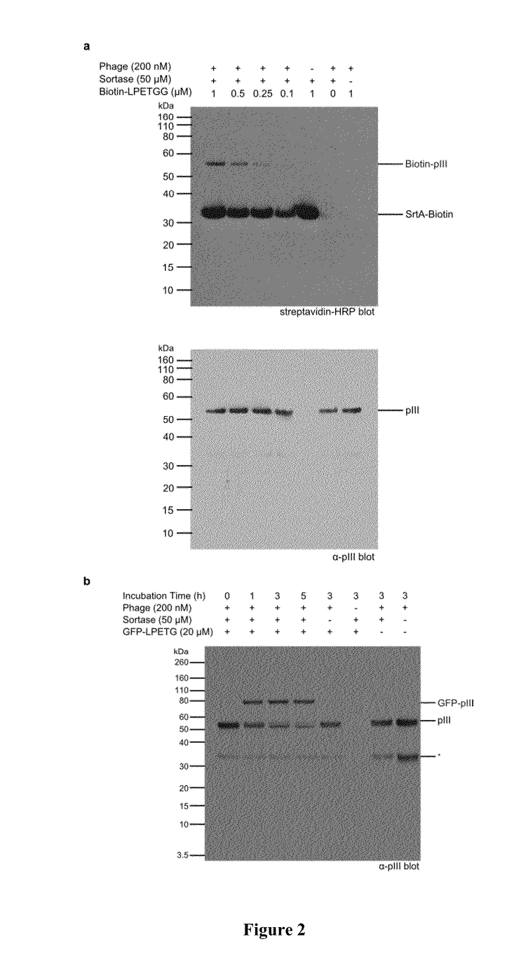

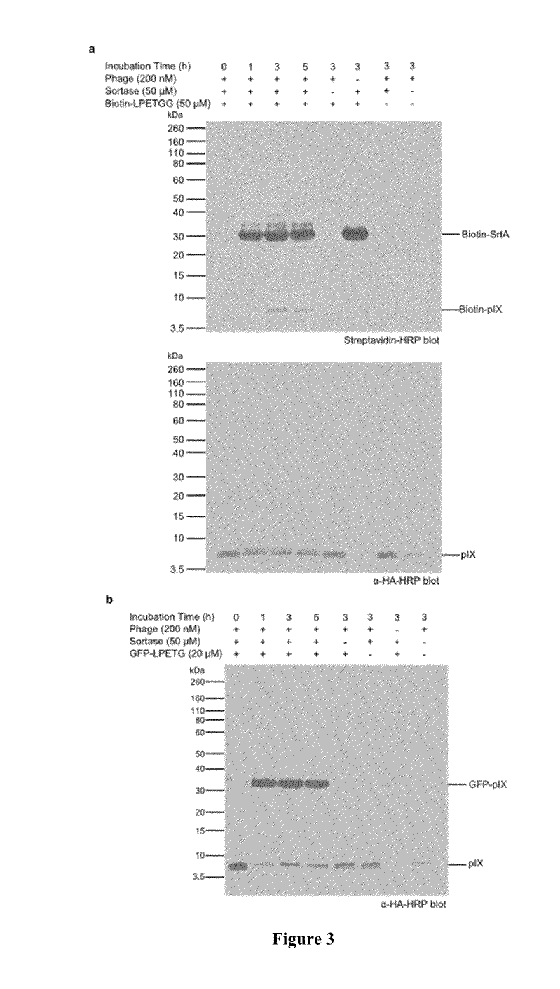

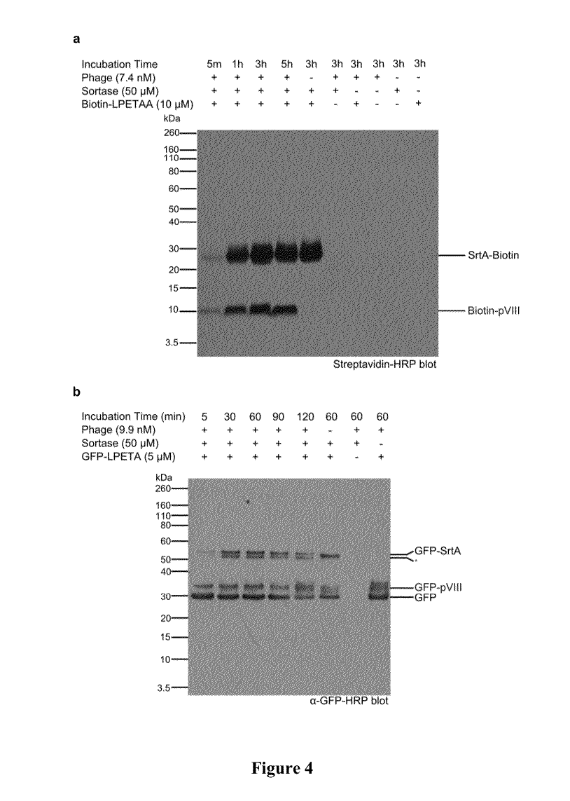

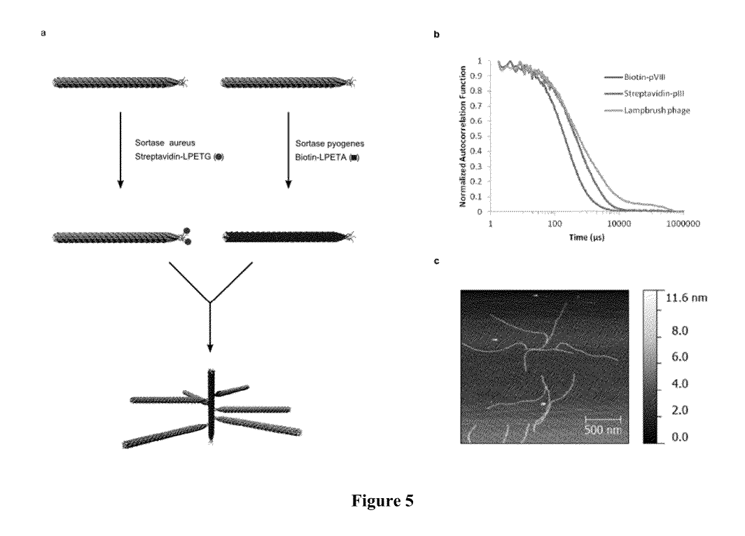

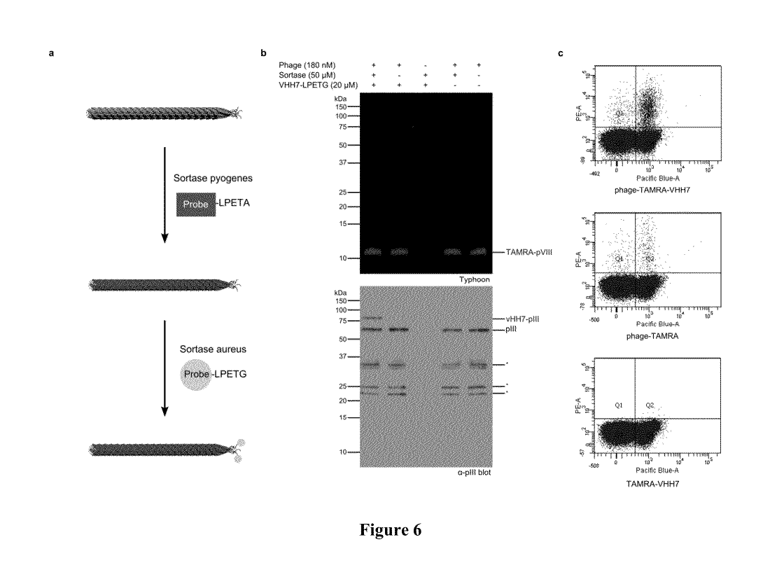

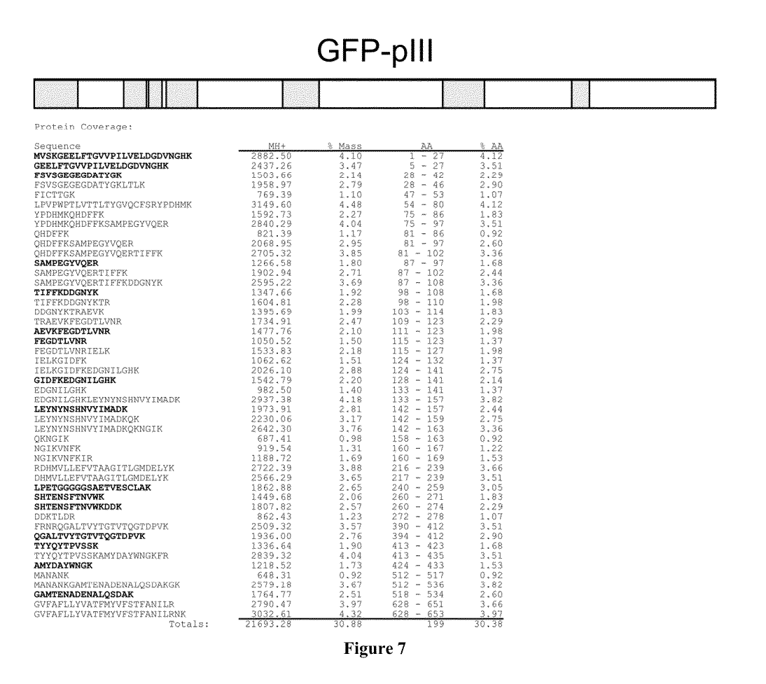

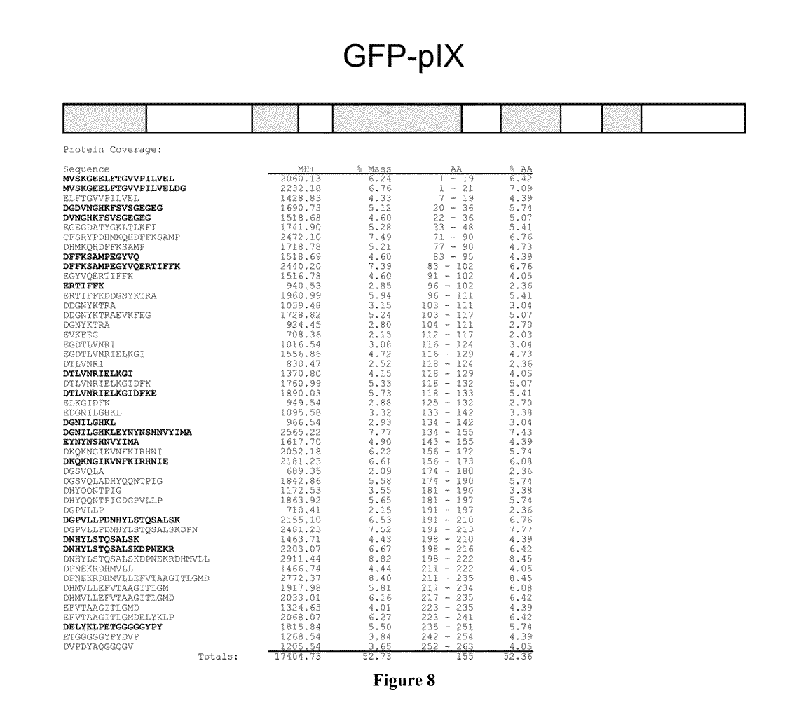

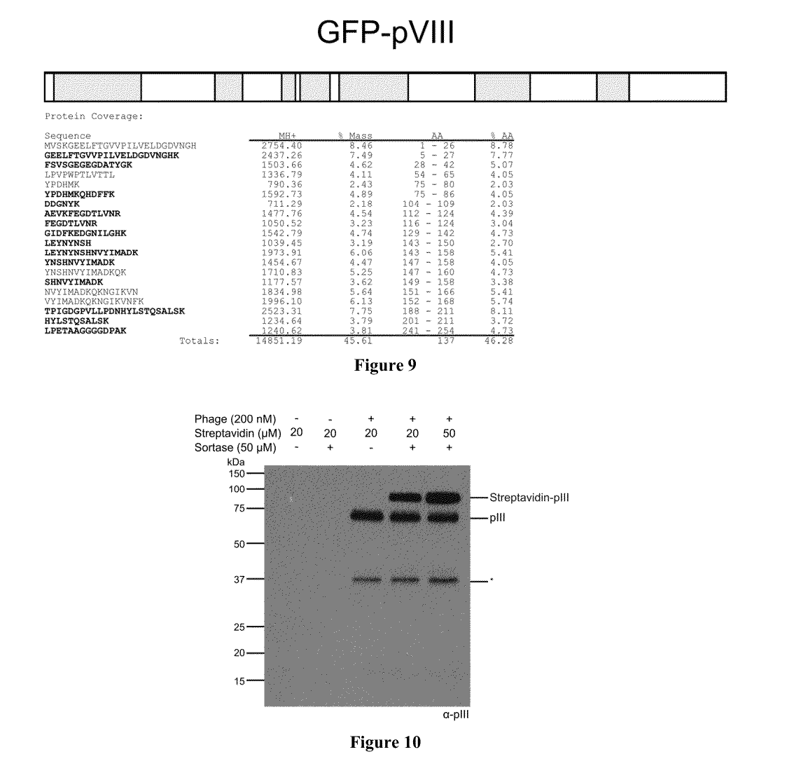

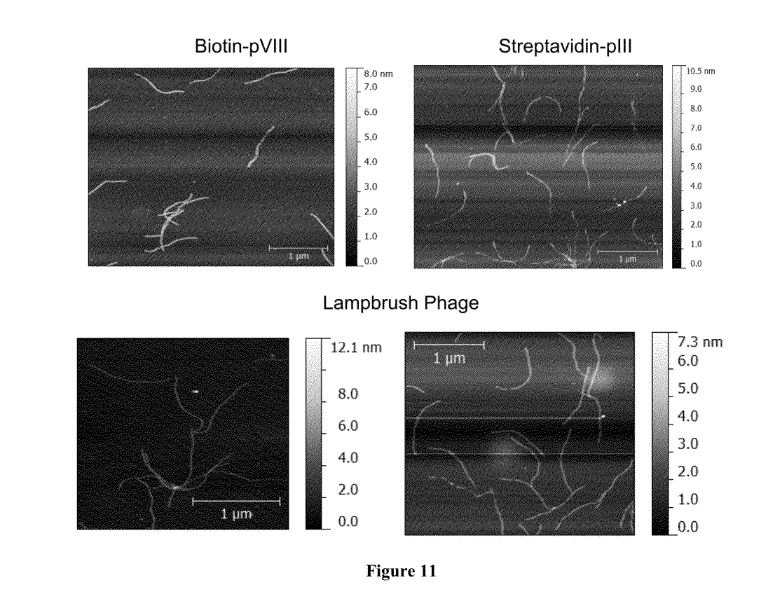

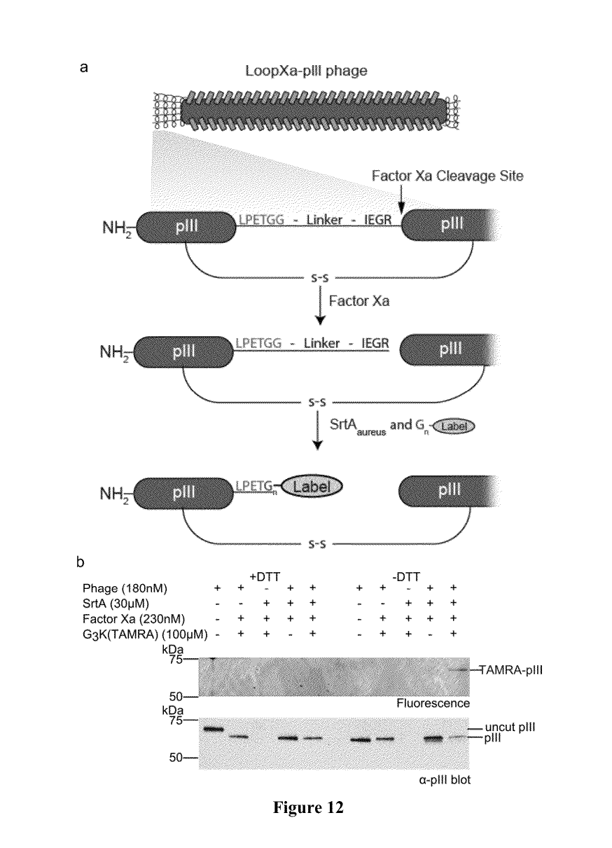

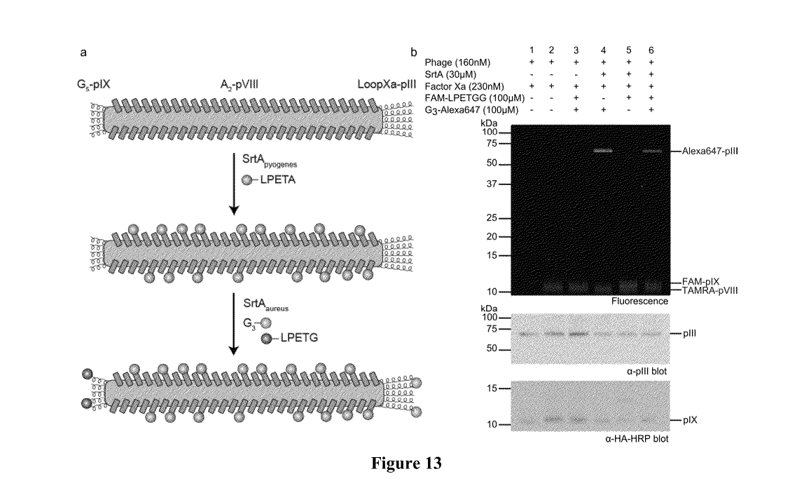

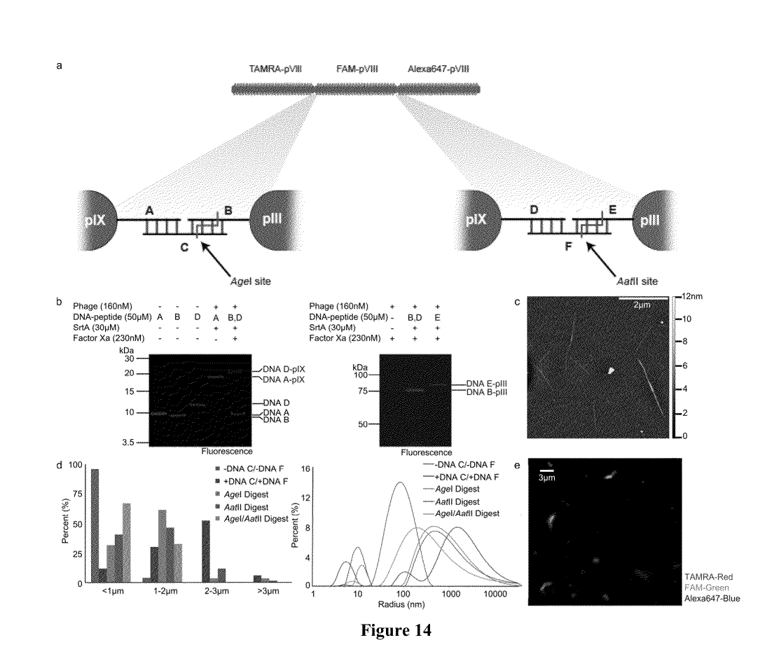

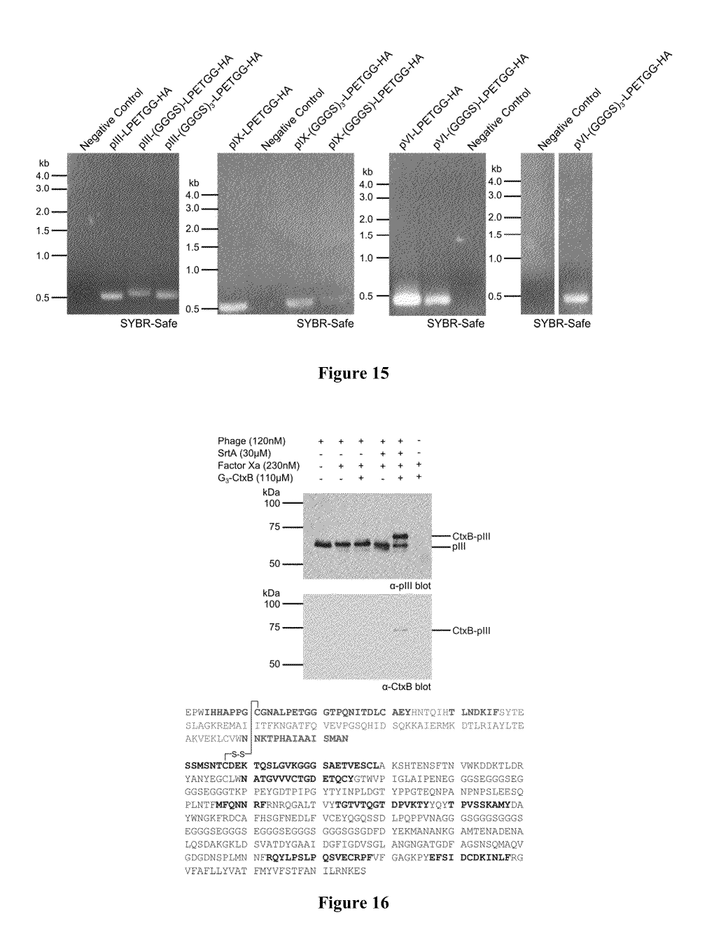

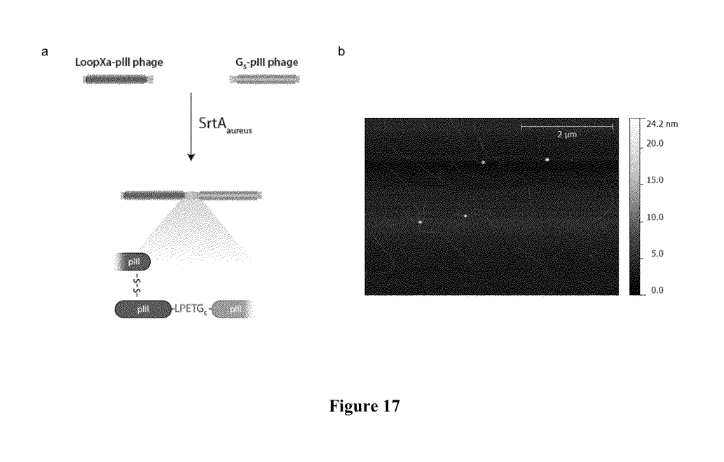

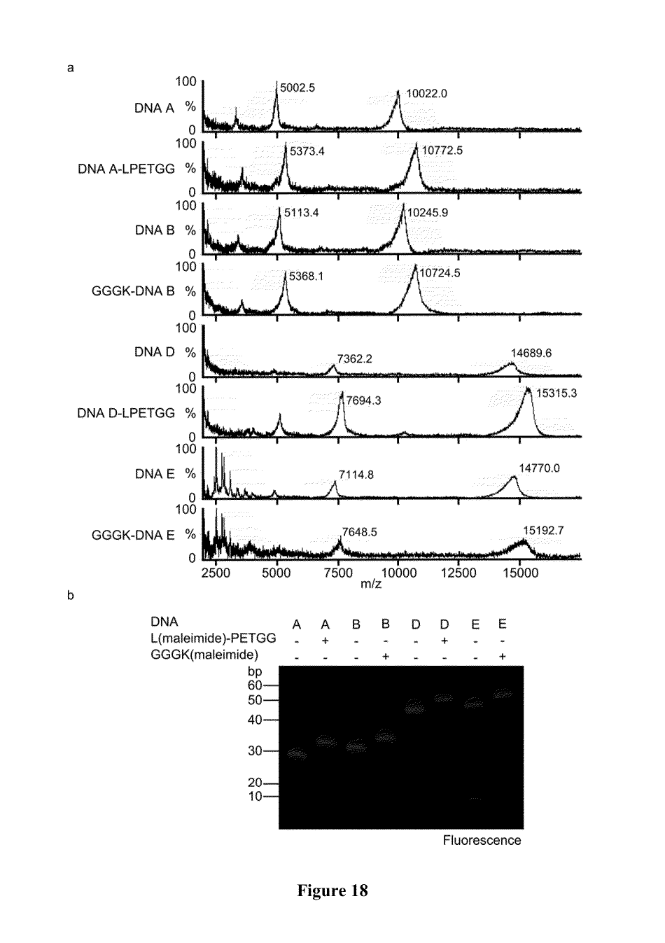

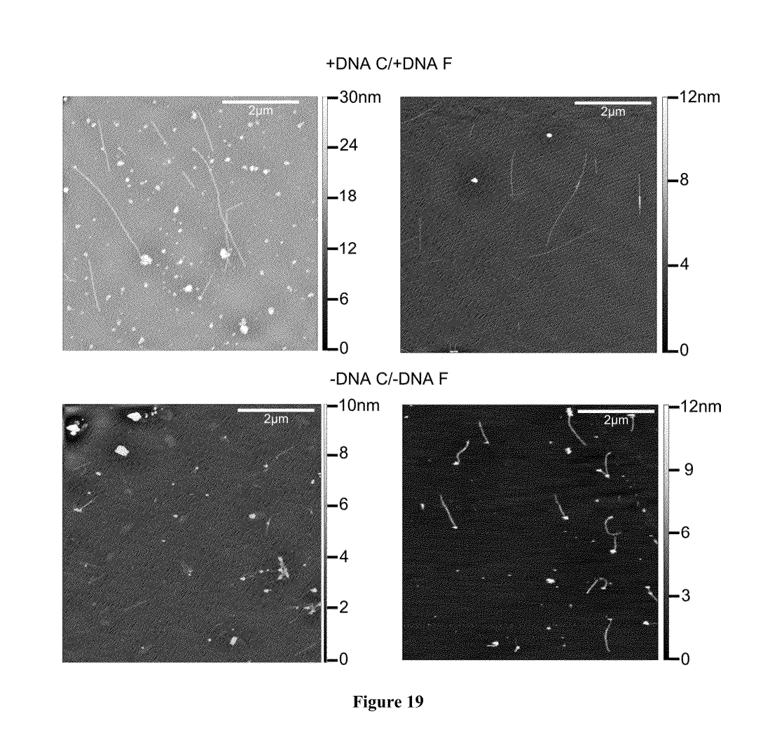

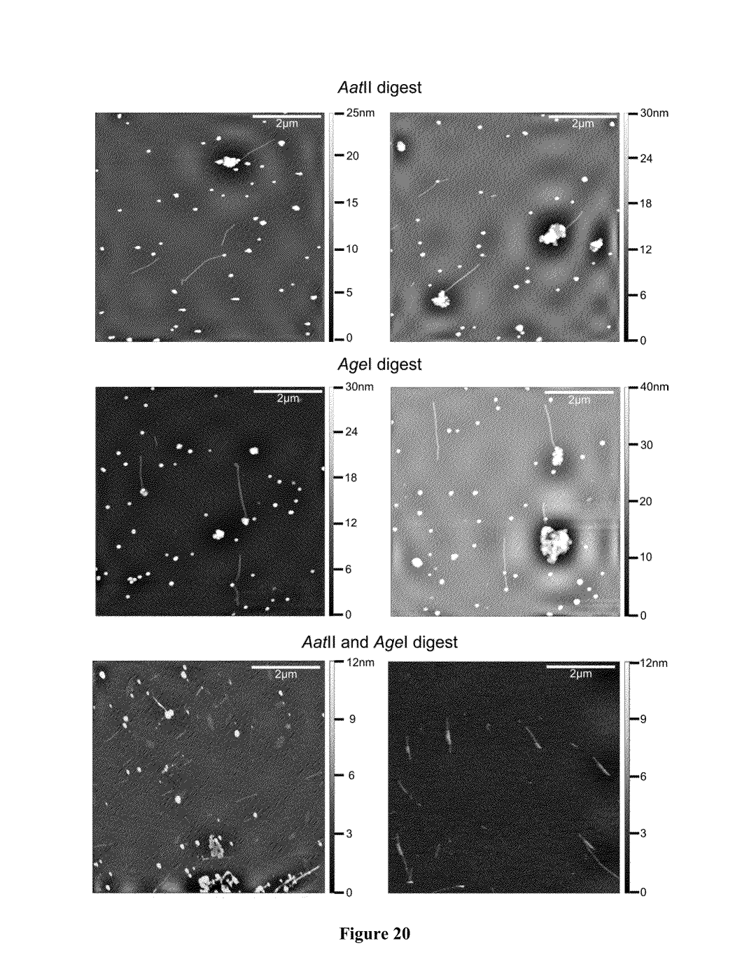

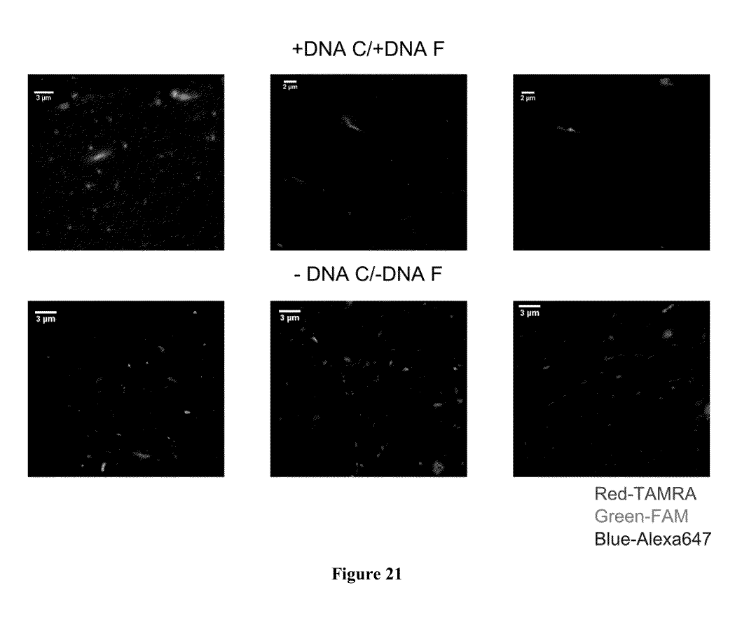

P111 has been the most extensively explored of the M13 capsid proteins in phage display because of the flexibility and accessibility of its N-terminus⁴⁶. Thus, we introduced five glycines at the N-terminus of pIII (G₅-pIII phage) (SEQ ID NO: 77) and used SrtAₐᵤᵣₑᵤₛ to covalently attach a K(biotin)-LPETGG peptide (SEQ ID NO: 13) (FIG. 2A). The biotin moiety allowed us to monitor the reaction by immunoblot analysis using streptavidin-HRP. Only when sortase, G₅-pIII phage (SEQ ID NO: 77), and the peptide are incubated together did we detect a 55 kDa streptavidin and anti-pIII reactive protein band (FIG. 2A). The reaction was specific: no other phage proteins were biotinylated. After 3 hrs at 37° C., we achieved a yield of 68±9% labeling using 50 μM peptide, 50 μM SrtAₐᵤᵣₑᵤₛ, 200 nM G₅-pIII phage (SEQ ID NO: 77), and 10 mM CaCl₂. The efficiency of the reaction was calculated using densitometric analysis of immunoblots where we compared the signal of the biotinylated pIII to biotinylated GFP standards of known concentration. The amount of biotinylated pIII was then divided by the amount of pIII molecules loaded onto the gel, as determined by UV-vis spectrometry. The quantification was repeated for three independent reactions with three samples analyzed for each reaction. The method of quantification is described in further detail in the Experimental Procedures section.

To determine whether sortase could be exploited to attach pre-folded proteins onto pIII, we used GFP containing an LPETG (SEQ ID NO: 10) motif at its C-terminus as a substrate. The reaction was analyzed by immunoblot using an anti-pIII antibody (FIG. 2B). Upon completion of the reaction, a mobility shift of pIII to the ˜80 kDa region, corresponding to the GFP-pIII fusion product, was detected. The identity of this material was confirmed by mass spectrometry (FIG. 2B and FIG. 7). After 3 hrs at room temperature, we achieved a yield of 56±2% labeling using 20 μM GFP-LPETG (SEQ ID NO: 10), 50 μM SrtAₐᵤᵣₑᵤₛ, 200 nM G₅-pIII phage, and 10 mM CaCl₂. The reaction was quantified by densitometry comparing the signal of pIII-GFP to the signal of the intact pIII input loaded into the reaction.

N-Terminal Labeling of pIX Using SrtAₐᵤᵣₑᵤₛ.

Because the C-terminus of pIX is buried in the phage structure and therefore unavailable for labeling⁴⁷, we attempted to label its N-terminus. However, this region of the protein is not as accessible as in pIII and our first attempts at labeling a phage construct displaying five glycines at the N-terminus of pIX using sortase failed (data not shown). To increase accessibility of the five glycines, the N-terminus of pIX was extended with an HA tag, a useful handle for detection, as no pIX-specific antibodies are available. This G₅HA-pIX (SEQ ID NO: 282) phage construct was labeled with the K(biotin)-LPETGG peptide (SEQ ID NO: 13) and the reactions were analyzed by immunoblot using streptavidin-HRP and an anti-HA antibody. A 5 kDa polypeptide, reactive with both streptavidin and anti-HA, was seen only in the complete reaction (FIG. 3A). We achieved a yield of 73±2% using 50 μM peptide, 50 μM SrtAₐᵤᵣₑᵤₛ, 200 nM G₅HA-pIX phage (SEQ ID NO: 282), and 10 mM CaCl₂ upon incubation at 37° C. for 3 hrs. A similar efficiency was attained when attaching GFP to pIX: 74±1% of pIX was labeled when 20 μM GFP-LPETG (SEQ ID NO: 10), 50 μM SrtAₐᵤᵣₑᵤₛ, 200 nM G₅HA-pIX phage (SEQ ID NO: 282), and 10 mM CaCl₂ were incubated for 3 hrs at room temperature. A 35 kDa anti-HA reactive polypeptide—consistent with the molecular mass of the GFP-pIX fusion protein—was detected only in the complete reaction and its identity was confirmed by mass spectrometry (FIG. 3B and FIG. 8).

N-Terminal Labeling of pVIII Using SrtA_{pyogenes}.

In the course of phage biogenesis the N-terminus of pVIII is proteolytically cleaved, resulting in the display of an N-terminal alanine⁴¹. We took advantage of this feature and exploited SrtA_{pyogenes} to label pVIII. Also, the ability of using two orthogonal sortase enzymes (SrtA_{pyogenes} for pVIII and SrtAₐᵤᵣₑᵤₛ for pIII and pIX labeling) would further enable dual labeling of the same phage particle.

To be used as a nucleophile in SrtA_{pyogenes}-mediated reactions, pVIII requires display of two N-terminal alanines. Thus, the N-terminus of the mature form of pVIII was modified to AAGGGG (A₂G₄-pVIII phage) (SEQ ID NO: 9). The glycines were introduced to extend the N-terminus of pVIII away from the body of the phage, thus improving the accessibility of the Ala-Ala motif for participation in the sortase reaction. Using SrtA_{pyogenes} and a K(biotin)-LPETAA (SEQ ID NO: 12) substrate peptide, we showed robust labeling of pVIII based on an immunoblot using streptavidin-HRP (FIG. 4A). Only when A₂G₄-pVIII (SEQ ID NO: 9) phage, SrtA_{pyogenes}, and the peptide were mixed together did we detect a biotinylated 10 kDa protein, consistent with the size of pVIII. The labeling reaction was site-specific as no other proteins can be detected in the blot. We obtained a yield of 50±3% labeled pVIII when reactions were performed at 37° C. for 3 hrs with 20 μM peptide, 50 μM SrtA_{pyogenes}, and 8 nM A₂G₄-pVIII phage (SEQ ID NO: 9). This translated to 1350±90 biotin molecules on average per phage particle.

Phage assembly limits either the size of the modifications displayed on pVIII to a few residues when using a phage vector, or it limits the number of labels attached to pVIII when using a phagemid vector²⁰. In this context, the sortase-labeling strategy is an obvious alternative to overcome such limitations. Using 20 μM GFP containing a LPETA (SEQ ID NO: 11) motif at its C-terminus, 50 μM SrtA_{pyogenes}, and 8 nM A₂G₄-pVIII phage (SEQ ID NO: 9), we were able to attach 91±20 GFP molecules on average per phage particle upon incubation at 37° C. for 3 hrs (FIG. 4B). The identity of the 35 kDa anti-GFP reactive protein, consistent with the size of a GFP-pVIII fusion protein, was confirmed by mass spectrometry (FIG. 4B and FIG. 9). As estimated by nearest neighbor packing, a single virion can accommodate 385 copies of GFP on its surface. Thus, using the sortase-mediated reaction, we obtained a yield of ˜25% of estimated maximum packing.

Building End-to-Body Phage Structures.

The ability to site-specifically label the M13 capsid proteins provides the opportunity to create novel multi-phage structures, which may provide scaffolds for new materials and devices. One such structure (FIG. 5A) relies on tight binding of the ends of several phage particles (via either pIII or pIX) to the body of another single phage (onto pVIII). However, direct covalent attachment between two phage proteins is not possible using sortase as we were unable to label the C-terminus of pIII, pIX, or pVIII (data not shown). This issue was solved by attaching streptavidin to pIII, biotin to pVIII, and then mixing the two preparations.

Streptavidin, modified to contain a C-terminal LPETG (SEQ ID NO: 10) motif in each of its monomers, was attached to the G₅-pIII (SEQ ID NO: 77) phage using SrtAₐᵤᵣₑᵤₛ. The samples were boiled, loaded onto an SDS-PAGE gel, and analyzed by immunoblot using an anti-pIII antibody. A 90 kDa polypeptide, consistent with the size of pIII fused to a streptavidin monomer, was seen only when all the reaction components were mixed together (FIG. 10). The streptavidin-pIII phage was purified from sortase and free streptavidin by PEG/NaCl precipitation. Dynamic light scattering (DLS) was performed in order to monitor dispersity and aggregation. The normalized autocorrelation function (ACF) of streptavidin-pIII phage showed an exponential decay consistent with monodisperse populations (FIG. 5B). This was confirmed by atomic force microscopy (AFM) that showed individual virions, indicating that only a single phage particle was attached per streptavidin tetramer (FIG. 11). Biotin was conjugated to pVIII using the K(biotin)-LPETAA peptide (SEQ ID NO: 12) and SrtA_{pyogenes} as described above. The biotinylated phage was purified by PEG/NaCl precipitation to remove free peptide and the sortase-acyl intermediate. The biotinylated phage was observed as individual phage particles by AFM and the ACF showed an exponential decay, again indicating a monodisperse population (FIG. 5B and FIG. 11).

The streptavidin-pIII phage and the biotin-pVIII phage were mixed at a 5:1 molar ratio and incubated at room temperature for 15 min. Analysis of these samples by DLS showed an increase of the hydrodynamic diameter for the lampbrush phage mixture (700 nm) when compared to streptavidin-pIII (516 nm) and biotin-pVIII (204 nm) phage preparations. When the two types of phage were mixed, the ACF (FIG. 5B) shows a rising shoulder at longer relaxation times, indicating a polydisperse population. The longer relaxation times observed in the shoulder represent structures larger than single phage. These larger structures were observed by AFM (FIG. 5C and FIG. 11). Linkages between the end of one phage and the body of another phage were observed when streptavidin-pIII and biotin-pVIII are mixed. These linkages were not detected when the individual phages were visualized by AFM (FIG. 11).

Site-Specific Labeling of Two Capsid Proteins in the Same Phage Particle.

The two orthogonal sortases used to label different capsid proteins offer the possibility to attach different moieties to the body (using SrtA_{pyogenes}) and to the end of phage (using SrtAₐᵤᵣₑᵤₛ) within the same virion. In such a strategy, either pIII or pIX could be labeled with SrtAₐᵤᵣₑᵤₛ orthogonally to the pVIII, so as a proof-of-concept, a phage variant that contains a double alanine at the N-terminus of pVIII and the pentaglycine motif at the N-terminus of pIII was generated (this construct is referred to as G₅-pIII-A₂-pVIII (SEQ ID NO: 77)). Conditions were optimized to label each of these proteins in a site-specific manner. Because such dual-labeled phage could be a useful tool to sort cells by FACS (see below and discussion section), we here provide the proof-of-concept by labeling the body of phage with a fluorophore and the tip of phage with a cell-targeting moiety.

pVIII was labeled with a K(TAMRA)-LPETAA (SEQ ID NO: 12) peptide and purified using PEG/NaCl precipitation to remove free peptide and sortase (FIG. 6A). A fluorescent 10 kDa protein, corresponding to pVIII, was the only polypeptide detected in the complete reaction. This confirmed successful labeling and site-specificity of SrtA_{pyogenes}. The pIII of this fluorescent phage was then incubated with SrtAₐᵤᵣₑᵤₛ and a 15 kDa single domain antibody, VHH7, modified with a C-terminal LPETG (SEQ ID NO: 10) motif. VHH7 recognizes murine Class II MHC products (the development and expression of VHH7 will be described elsewhere). Attachment of VHH7 to pIII was monitored by immunoblot using an anti-pIII antibody (FIG. 6B). Comparing the signal intensities of VHH7-pIII 90 kDa polypeptide and of pIII, we estimated that on average 2-3 VHH7 molecules are attached per phage particle, a number similar to what can be obtained when screening phagemid libraries of pIII fusions by panning⁴⁸⁻⁴⁹.

Flow Cytometry Experiments Using Fluorescent Phage.

Fluorescent phage has been used for targeted staining in vivo⁵⁰⁻⁵¹ as well as flow cytometry experiments⁵². However, these have been performed with short peptide phage display libraries. The ability to label phage with a large number of fluorophores that are site-specifically attached to pVIII is a tool useful for selecting phage of interest from phage display libraries of large moieties (such as antibodies) by fluorescence. With libraries of this type, less specific labeling methods can alter the displayed moiety. To provide proof-of-concept that fluorescent phage can be used for this purpose, we tested the ability of the dual labeled phage—containing TAMRA fluorophore sortagged onto pVIII and VHH7 onto pIII—to stain B cells. As a negative control, we used a fluorescent phage containing an anti-GFP VHH attached to pIII⁵³. An average yield of 2.5 antibodies per phage virion was achieved for both VHH7 and anti-GFP VHH as determined by densitometric analysis.

Mouse lymphocytes obtained from lymph nodes were stained for B cells using a fluorescent Pacific Blue anti-mouse B220 antibody and incubated with phage-VHH7, phage-anti-GFP, or non-targeted phage. All phage preparations were similarly labeled with TAMRA on pVIII. After removal of unbound materials by washing, cells were subjected to flow cytometry (FIG. 6C). When stained with phage-VHH7, we detected an increase in cells double positive for TAMRA and the B cell marker compared to non-specific staining with phage-anti-GFP or non-targeted phage. Staining of cells with phage-VHH7 was vastly superior to VHH7 directly conjugated to TAMRA, as only a few double positive cells were detected when incubated with an equivalent amount of the latter (FIG. 6C).

### Discussion

We show that sortase-mediated reactions overcome many of the limitations of current methods to functionalize M13 capsid proteins. The main body and both ends of the viral capsid can be functionalized with substituents that cannot be encoded genetically (such as biotin and fluorophores), and we can also install properly folded and assembled proteins (such as GFP and streptavidin) in a manner that could easily be extended to oligomeric proteins as well.

One of the major challenges has been the modification of the major capsid protein pVIII. Using sortase, labeling efficiencies were greater than those obtained genetically (Table 4). In the past, biotinylated phage has been produced by display of the biotin acceptor peptide (BAP)⁵⁴, a 15-amino acid sequence. Peptides similar in size have been displayed at no more than 400-700 copies per phage, with the efficiency being sequence-dependent²⁰. Here we attach 1350 biotin molecules on average per phage particle, a great improvement in the display of a small molecule. Moreover, because the peptide substrate for sortase can be modified with peptides, proteins, fluorophores, etc.³¹⁻³⁵, phage can be decorated with a wide range of substituents. As far as display of proteins is concerned, proteins similar in size to GFP have been incorporated at fewer than one copy per phage on pVIII using a phagemid system¹⁸. Using sortase, we display 91 GFP molecules on average per phage particle.

TABLE 4
Labeling efficiency for each of the
phage coat proteins using sortase.
Minor Capsid Proteins

| Capsid Protein | Probe | Efficiency |
|---|---|---|
| pIII | Biotin | 68 ± 9% |
| pIII | GFP | 56 ± 2% |
| pIX | Biotin | 73 ± 2% |
| pIX | GFP | 74 ± 1% |

Major Capsid Protein

| | | Optimal | Copy Number/Phage | Liter- |
|---|---|---|---|---|
| Capsid Protein | Probe | Packing | Using Sortase | ature |
| pVIII | Biotin | 2700 | 1350 ± 90 | 400-700 |
| pVIII | GFP | 385 | 91 ± 20 | <1 |

For the pIII and pIX proteins, we show that every phage can be labeled with multiple copies of the desired peptide/protein (Table 4). An advantage of using sortase to covalently attach proteins to phage over genetically engineering pIII directly is that it ensures display of the correct quaternary structure of the protein. This can be inferred from our experiments using streptavidin. The mixing of two phage particles, one containing streptavidin on pIII and the other containing biotin on pVIII results in a novel and complex phage structure. This shows that the streptavidin structure displayed on phage remains fully active and binds biotin.

Sortase enzymes in combination with the streptavidin-biotin pair⁴⁵ or in conjunction with click-chemistry can generate novel structures. The ability of patterning and aligning materials on phage or of increasing its surface area is important for the development of new materials. For example, the lampbrush phage structure generated here (FIG. 5) may find application in light-sensitive processes where phage branching off the stem could be functionalized to act as antennae to capture light⁵⁵.

In addition to N-terminal labeling of single capsid proteins, two capsid proteins were labeled site-specifically on a single phage particle using two orthogonal sortases. This could be explored for panning of antibody libraries displayed on pIII. Due to the exquisite site-specificity of sortase, fluorescent peptides can be added to pVIII without modification of the moiety displayed at pIII. Fluorescent labeling by other chemistries does not easily afford such specificity, especially when displaying a large moiety, such as an antibody fragment. The sensitivity of detection should increase when a phage particle contains many fluorophore groups on pVIII. This is indeed what we observe in our flow cytometry experiments, showing that this strategy greatly enhances the sensitivity of detection. Increased sensitivity would be instrumental in the context of a future panning strategy for detection of rare binding events, whether due to low concentration of the target or low phage concentration.

Modification of pIII and pIX by sortase will be useful for material applications, where the physical properties of phage and not its utility as a library vector are of prime concern. Fluorescent modification of pVIII is compatible with the construction and screening of libraries created using pIII genetic fusions. In this case, the site-specificity and yield of the sortase reaction allow the generation of libraries that can be screened directly by fluorescence. Thus, the versatility of the sortase-based labeling strategy described here will enable development of a wide array of tools, expanding the use of phage either for the creation of new materials or for new biological applications.

### REFERENCES

(1) Sidhu, S. S. (2001) Engineering M13 for phage display. Biomol. Eng. 18, 57-63.
(2) Bratkovic, T. (2010) Progress in phage display: evolution of the technique and its application. Cell. Mol. Life. Sci. 67, 749-67.
(3) Burritt, J. B., Quinn, M. T., Jutila, M. A., Bond, C. W., and Jesaitis, A. J. (1995) Topological mapping of neutrophil cytochrome b epitopes with phage-display libraries. J. Biol. Chem. 270, 16974-80.
(4) Barry, M. A., Dower, W. J., and Johnston, S. A. (1996) Toward cell-targeting gene therapy vectors: selection of cell-binding peptides from random peptide-presenting phage libraries. Nat. Med. 2, 299-305.
(5) Jaye, D. L., Nolte, F. S., Mazzucchelli, L., Geigerman, C., Akyildiz, A., and Parkos, C. A. (2003) Use of real-time polymerase chain reaction to identify cell- and tissue-type-selective peptides by phage display. Am. J. Pathol. 162, 1419-29.
(6) Mazzucchelli, L., Burritt, J. B., Jesaitis, A. J., Nusrat, A., Liang, T. W., Gewirtz, A. T., Schnell, F. J., and Parkos, C. A. (1999) Cell-specific peptide binding by human neutrophils. Blood 93, 1738-48.
(7) Whaley, S. R., English, D. S., Hu, E. L., Barbara, P. F., and Belcher, A. M. (2000) Selection of peptides with semiconductor binding specificity for directed nanocrystal assembly. Nature 405, 665-8.
(8) Udit, A. K., Hollingsworth, W., and Choi, K. (2010) Metal- and metallocycle-binding sites engineered into polyvalent virus-like scaffolds. Bioconjug Chem 21, 399-404.
(9) Mao, C., Flynn, C. E., Hayhurst, A., Sweeney, R., Qi, J., Georgiou, G., Iverson, B., and Belcher, A. M. (2003) Viral assembly of oriented quantum dot nanowires. Proc. Natl. Acad. Sci. U.S.A. 100, 6946-51.
(10) Mao, C., Solis, D. J., Reiss, B. D., Kottmann, S. T., Sweeney, R. Y., Hayhurst, A., Georgiou, G., Iverson, B., and Belcher, A. M. (2004) Virus-based toolkit for the directed synthesis of magnetic and semiconducting nanowires. Science 303, 213-7.
(11) Nam, K. T., Kim, D. W., Yoo, P. J., Chiang, C. Y., Meethong, N., Hammond, P. T., Chiang, Y. M., and Belcher, A. M. (2006) Virus-enabled synthesis and assembly of nanowires for lithium ion battery electrodes. Science 312, 885-8.
(12) Nam, Y. S., Magyar, A. P., Lee, D., Kim, J. W., Yun, D. S., Park, H., Pollom, T. S., Jr., Weitz, D. A., and Belcher, A. M. (2010) Biologically templated photocatalytic nanostructures for sustained light-driven water oxidation. Nat. Nanotechnol. 5, 340-4.
(13) Dang, X., Yi, H., Ham, M. H., Qi, J., Yun, D. S., Ladewski, R., Strano, M. S., Hammond, P. T., and Belcher, A. M. (2011) Virus-templated self-assembled single-walled carbon nanotubes for highly efficient electron collection in photovoltaic devices. Nat. Nanotechnol. 6, 377-84.
(14) Ng, S., Jafari, M. R., and Derda, R. (2011) Bacteriophages and viruses as a support for organic synthesis and combinatorial chemistry. ACS Chem. Biol. 7, 123-38.
(15) Kaltgrad, E., O'Reilly, M. K., Liao, L., Han, S., Paulson, J. C., and Finn, M. G. (2008) On-virus construction of polyvalent glycan ligands for cell-surface receptors. J. Am. Chem. Soc. 130, 4578-9.
(16) Lee, Y. J., Yi, H., Kim, W. J., Kang, K., Yun, D. S., Strano, M. S., Ceder, G., and Belcher, A. M. (2009) Fabricating genetically engineered high-power lithium-ion batteries using multiple virus genes. Science 324, 1051-5.
(17) Bianchi, E., Folgori, A., Wallace, A., Nicotra, M., Acali, S., Phalipon, A., Barbato, G., Bazzo, R., Cortese, R., Felici, F., and et al. (1995) A conformationally homogeneous combinatorial peptide library. J. Mol. Biol. 247, 154-60.
(18) Corey, D. R., Shiau, A. K., Yang, Q., Janowski, B. A., and Craik, C. S. (1993) Trypsin display on the surface of bacteriophage. Gene 128, 129-34.
(19) Kang, A. S., Barbas, C. F., Janda, K. D., Benkovic, S. J., and Lerner, R. A. (1991) Linkage of recognition and replication functions by assembling combinatorial antibody Fab libraries along phage surfaces. Proc. Natl. Acad. Sci. U.S.A. 88, 4363-6.
(20) Malik, P., Terry, T. D., Gowda, L. R., Langara, A., Petukhov, S. A., Symmons, M. F., Welsh, L. C., Marvin, D. A., and Perham, R. N. (1996) Role of capsid structure and membrane protein processing in determining the size and copy number of peptides displayed on the major coat protein of filamentous bacteriophage. J. Mol. Biol. 260, 9-21.
(21) Markland, W., Roberts, B. L., Saxena, M. J., Guterman, S. K., and Ladner, R. C. (1991) Design, construction and function of a multicopy display vector using fusions to the major coat protein of bacteriophage M13. Gene 109, 13-9.
(22) Bass, S., Greene, R., and Wells, J. A. (1990) Hormone phage: an enrichment method for variant proteins with altered binding properties. Proteins 8, 309-14.
(23) Sidhu, S. S., Weiss, G. A., and Wells, J. A. (2000) High copy display of large proteins on phage for functional selections. J. Mol. Biol. 296, 487-95.
(24) Kretzschmar, T. and Geiser, M. (1995) Evaluation of antibodies fused to minor coat protein III and major coat protein VIII of bacteriophage M13. Gene 155, 61-5.
(25) Greenwood, J., Willis, A. E., and Perham, R. N. (1991) Multiple display of foreign peptides on a filamentous bacteriophage. Peptides from Plasmodium falciparum circumsporozoite protein as antigens. J. Mol. Biol. 220, 821-7.
(26) Iannolo, G., Minenkova, O., Petruzzelli, R., and Cesareni, G. (1995) Modifying filamentous phage capsid: limits in the size of the major capsid protein. J. Mol. Biol. 248, 835-44.
(27) Gao, C., Mao, S., Lo, C. H., Wirsching, P., Lerner, R. A., and Janda, K. D. (1999) Making artificial antibodies: a format for phage display of combinatorial heterodimeric arrays. Proc. Natl. Acad. Sci. U.S.A. 96, 6025-30.
(28) Jespers, L. S., Messens, J. H., De Keyser, A., Eeckhout, D., Van den Brande, I., Gansemans, Y. G., Lauwereys, M. J., Vlasuk, G. P., and Stanssens, P. E. (1995) Surface expression and ligand-based selection of cDNAs fused to filamentous phage gene VI. Biotechnology 13, 378-82.
(29) Georgieva, Y. and Konthur, Z. (2011) Design and screening of M13 phage display cDNA libraries. Molecules 16, 1667-81.
(30) Zozulya, S., Lioubin, M., Hill, R. J., Abram, C., and Gishizky, M. L. (1999) Mapping signal transduction pathways by phage display. Nat. Biotechnol. 17, 1193-8.
(31) Guimaraes, C. P., Carette, J. E., Varadarajan, M., Antos, J., Popp, M. W., Spooner, E., Brummelkamp, T. R., and Ploegh, H. L. (2011) Identification of host cell factors required for intoxication through use of modified cholera toxin. J. Cell Biol. 195, 751-64.
(32) Popp, M. W., Dougan, S. K., Chuang, T. Y., Spooner, E., and Ploegh, H. L. (2011) Sortase-catalyzed transformations that improve the properties of cytokines. Proc. Natl. Acad. Sci. U.S.A. 108, 3169-74.
(33) Antos, J. M., Chew, G. L., Guimaraes, C. P., Yoder, N. C., Grotenbreg, G. M., Popp, M. W., and Ploegh, H. L. (2009) Site-specific N- and C-terminal labeling of a single polypeptide using sortases of different specificity. J. Am. Chem. Soc. 131, 10800-1.
(34) Antos, J. M., Miller, G. M., Grotenbreg, G. M., and Ploegh, H. L. (2008) Lipid modification of proteins through sortase-catalyzed transpeptidation. J. Am. Chem. Soc. 130, 16338-43.
(35) Popp, M. W., Antos, J. M., Grotenbreg, G. M., Spooner, E., and Ploegh, H. L. (2007) Sortagging: a versatile method for protein labeling. Nat. Chem. Biol. 3, 707-8.
(36) Ton-That, H., Liu, G., Mazmanian, S. K., Faull, K. F., and Schneewind, O. (1999) Purification and characterization of sortase, the transpeptidase that cleaves surface proteins of Staphylococcus aureus at the LPXTG motif. Proc. Natl. Acad. Sci. U.S.A. 96, 12424-9.
(37) Ton-That, H., Mazmanian, S. K., Faull, K. F., and Schneewind, O. (2000) Anchoring of surface proteins to the cell wall of Staphylococcus aureus. Sortase catalyzed in vitro transpeptidation reaction using LPXTG peptide and NH(2)-Gly(3) substrates. J. Biol. Chem. 275, 9876-81.
(38) Popp, M. W. and Ploegh, H. L. (2011) Making and breaking peptide bonds: protein engineering using sortase. Angew. Chem. Int. Ed. Engl. 50, 5024-32.
(39) Race, P. R., Bentley, M. L., Melvin, J. A., Crow, A., Hughes, R. K., Smith, W. D., Sessions, R. B., Kehoe, M. A., McCafferty, D. G., and Banfield, M. J. (2009) Crystal structure of Streptococcus pyogenes sortase A: implications for sortase mechanism. J. Biol. Chem. 284, 6924-33.
(40) Petrenko, V. A., Smith, G. P., Gong, X., and Quinn, T. (1996) A library of organic landscapes on filamentous phage. Protein Eng. 9, 797-801.
(41) Barbas, C. F., Burton, D. R., Scott, J. K., and Silverman, G. J. (2001) Phage Display: A Laboratory Manual. Cold Spring Harbor Laboratory Press: Cold Spring Harbor, N.Y.
(42) Popp, M. W., Antos, J. M., and Ploegh, H. L. (2009) Site-specific protein labeling via sortase-mediated transpeptidation. Curr. Protoc. Protein Sci. Chapter 15, Unit 15 3.
(43) Lee, C. V., Sidhu, S. S., and Fuh, G. (2004) Bivalent antibody phage display mimics natural immunoglobulin. J Immunol Methods 284, 119-32.
(44) Howarth, M., Chinnapen, D. J., Gerrow, K., Dorrestein, P. C., Grandy, M. R., Kelleher, N. L., El-Husseini, A., and Ting, A. Y. (2006) A monovalent streptavidin with a single femtomolar biotin binding site. Nat. Methods 3, 267-73.
(45) Matsumoto, T., Sawamoto, S., Sakamoto, T., Tanaka, T., Fukuda, H., and Kondo, A. (2011) Site-specific tetrameric streptavidin-protein conjugation using sortase A. J. Biotechnol. 152, 37-42.
(46) Lubkowski, J., Hennecke, F., Pluckthun, A., and Wlodawer, A. (1998) The structural basis of phage display elucidated by the crystal structure of the N-terminal domains of g3p. Nat. Struct. Biol. 5, 140-7.
(47) Makowski, L. (1992) Terminating a macromolecular helix. Structural model for the minor proteins of bacteriophage M13. J. Mol. Biol. 228, 885-92.
(48) O'Connell, D., Becerril, B., Roy-Burman, A., Daws, M., and Marks, J. D. (2002) Phage versus phagemid libraries for generation of human monoclonal antibodies. J. Mol. Biol. 321, 49-56.
(49) Rondot, S., Koch, J., Breitling, F., and Dubel, S. (2001) A helper phage to improve single-chain antibody presentation in phage display. Nat. Biotechnol. 19, 75-8.
(50) Kelly, K. A., Setlur, S. R., Ross, R., Anbazhagan, R., Waterman, P., Rubin, M. A., and Weissleder, R. (2008) Detection of early prostate cancer using a hepsin-targeted imaging agent. Cancer Res. 68, 2286-91.
(51) Kelly, K. A., Waterman, P., and Weissleder, R. (2006) In vivo imaging of molecularly targeted phage. Neoplasia 8, 1011-8.
(52) Jaye, D. L., Geigerman, C. M., Fuller, R. E., Akyildiz, A., and Parkos, C. A. (2004) Direct fluorochrome labeling of phage display library clones for studying binding specificities: applications in flow cytometry and fluorescence microscopy. J. Immunol. Methods 295, 119-27.
(53) Kirchhofer, A., Helma, J., Schmidthals, K., Frauer, C., Cui, S., Karcher, A., Pellis, M., Muyldermans, S., Casas-Delucchi, C. S., Cardoso, M. C., Leonhardt, H., Hopfner, K. P., and Rothbauer, U. (2010) Modulation of protein properties in living cells using nanobodies. Nat. Struct. Mol. Biol. 17, 133-8.
(54) Schatz, P. J. (1993) Use of peptide libraries to map the substrate specificity of a peptide-modifying enzyme: a 13 residue consensus peptide specifies biotinylation in Escherichia coli. Biotechnology 11, 1138-43.
(55) Nam, Y. S., Shin, T., Park, H., Magyar, A. P., Choi, K., Fantner, G., Nelson, K. A., and Belcher, A. M. (2010) Virus-templated assembly of porphyrins into light-harvesting nanoantennae. J. Am. Chem. Soc. 132, 1462-3.

All publications, patents, patent applications, and database entries mentioned anywhere herein, including, but not limited to, those items listed above, are hereby incorporated by reference in their entirety as if each individual publication, patent, patent application, and database entry was specifically and individually indicated to be incorporated by reference. In case of conflict, the present application, including any definitions herein, will control.

### Example 2

### Orthogonal Labeling of M13 Minor Capsid Proteins with DNA to Self-Assemble End-to-End Multi-Phage Structures

A major goal of synthetic biology is to control and program biological molecules to perform a desired function, such as the organization of materials to create devices.¹ In this context, the self-assembling capsid proteins of M13 bacteriophage have been explored to form nanowire structures,²⁻³ which have been used to build battery and solar devices.⁴⁻⁵ M13 bacteriophage is an attractive building block for more complex multi-material devices such as transistors and diodes, because its major capsid protein (pVIII) can been engineered to bind and nucleate different materials.^{2,4,6}

The building of more complex materials requires construction of multi-phage scaffolds, but this has been hampered by the inability to freely manipulate the major capsid protein located in the body of phage and the four minor capsid proteins located at the ends of the phage (pIII, pVI, pVII, pIX) to form specific connections between different M13 particles. Streptavidin-based conjugates⁶⁻⁸ and leucine zippers⁹ have been explored to connect virions through the pIII, pVIII, or pIX proteins, but the resultant structures neither displayed a 1:1 stoichiometry—as streptavidin can bind up to four biotin molecules—nor did they allow precise control over structure length.⁹

DNA hybridization is a commonly used strategy to establish nanoscale connections. It has been used to order spherical viruses¹⁰⁻¹¹ and order gold nanoparticles into crystal lattices.¹²⁻¹³ Although these and polymer-based particles can be conjugated with DNA¹⁴⁻¹⁵, the use of M13 offers two main advantages: high aspect ratio scaffolds and five proteins that may be engineered for different functions. Crosslinking individual M13 phage particles by means of DNA hybridization would have several advantages: first, a 1:1 stoichiometry with easier control over the number of phage coming together at a single connection; second, specificity and versatility, as the sequence of a DNA oligonucleotide can be modified to form new orthogonal complementary pairs; and third, reversible ligations, as DNA-DNA interactions can be disrupted by heat and reformed by cooling.

We accomplished specific labeling of the N-termini of pIII and pIX, with a variety of substituents using the sortase enzyme from Staphylococcus aureus (SrtAₐᵤᵣₑᵤₛ).⁷ Sortase-catalyzed transpeptidation reactions comprise two steps: initial recognition of an LPXTG (SEQ ID NO: 78) motif placed near the C-terminus of a polypeptide which SrtAₐᵤᵣₑᵤₛ cleaves after the threonine residue to form a thioester-linked acyl-enzyme intermediate. This is followed by a nucleophilic attack by the α-amine of an oligoglycine (poly)peptide, which resolves the intermediate. Because the LPXTG (SEQ ID NO: 78) motif-containing (poly)peptide can be conjugated beforehand with any substituent of choice (e.g., fluorophore), the final product is the protein of interest—in this case pIII or pIX—labeled at the N-terminus with that substituent. The SrtAₐᵤᵣₑᵤₛ catalyzed reactions are orthogonal to Streptococcus pyogenes sortase A (SrtA_{pyogenes})-mediated labeling of pVIII, as the enzyme recognizes an LPXTA (SEQ ID NO: 92) motif and the intermediate is resolved by an N-terminal double alanine nucleophile^{7,16} instead of the (Gly)ₙ preferred by SrtAₐᵤᵣₑᵤₛ.

Here we describe the installation of a loop structure comprising the LPXTG (SEQ ID NO: 78) sortase recognition motif on pIII to enable C-terminal display. Using an M13 construct containing three sortase labeling motifs within the same virion, we demonstrate orthogonal labeling of pIII, pVIII, and pIX proteins. Using this construct, we built end-to-end multi-phage structures in a specific order by labeling the pIII and pIX proteins with DNA and different fluorophores on the pVIII.

### Results and Discussion

C-Terminal Phage Vector Display of the Sortase Substrate Motif.

We first examined whether we could display the LPXTG (SEQ ID NO: 78) sortase-recognition motif at the C-terminus of the pIII, pVI, or pIX proteins. Although genetic engineering of the M13 phage genome yielded the desired modifications as confirmed by PCR (FIG. 15), they were incompatible with phage assembly. The DNA oligonucleotides used for phage engineering are shown in Table 6. We introduced unique enzyme restriction sites at the C-termini of pIII, pVI, and pIX coding sequences. We did not explore pVII and pVIII, as their C-termini seem to be even less exposed (Makowski, L., Terminating a macromolecular helix. Structural model for the minor proteins of bacteriophage M13. Journal of molecular biology 1992, 228 (3), 885-92). The template vector for all the cloning steps derives from the 983 vector (Ghosh, D.; Kohli, A. G.; Moser, F.; Endy, D.; Belcher, A. M., Refactored M13 Bacteriophage as a Platform for Tumor Cell Imaging and Drug Delivery. ACS Synthetic Biology 2012), and contained the biotin acceptor peptide (GLQDIFEAQKIEWHE (SEQ ID NO: 118)) fused to the N-terminus of pIX, DSPHTELP (SEQ ID NO: 119) on the N-terminus of pVIII, and SPARC (secreted protein, acidic and rich in cysteine) binding peptide (SPPTGINGGG (SEQ ID NO: 120)) on the N-terminus of pIII. Using site-directed mutagenesis, we inserted recognition sites for BclI and BspEI (oligonucleotides: pIII-BspEIBclITop and pIII-BspEIBclIBottom) on pIII, and AatII and AgeI (oligonucleotides: pVI-AatIITop and pVI-AatIIBottom, pVI-AgeITop and pVI-AgeIBottom) on pVI. A unique BspHI restriction site was readily available near the C-terminus of pIX and we engineered a SpeI site (oligonucleotides: pIX-SpeITop and pIX-SpeIBottom). Using the inserted restriction sites, we introduced an LPETGG (SEQ ID NO: 13) motif followed by an HA tag to the C-termini of the capsid proteins. By inserting no linker, GGGS (SEQ ID NO: 284), and (GGGS)₃ (SEQ ID NO: 285) immediately upstream the LPETGG motif (SEQ ID NO: 13), we extended its flexibility. We confirmed successful cloning by PCR, using a set of primers in which one of them anneals in the insert and the other elsewhere in the genome (FIG. 15). The PCR reactions were analyzed on a 1% agarose gel stained with SYBR Safe Stain (Life Technologies), and visualized using a Gel Doc 2000 Gel Documentation System (BioRad). We detected a ˜500 bp PCR product only when a primer annealing within the insertion was included. However, bacteria transformed with this ligation reaction showed no phage containing the modifications.

We then engineered the N-terminus of pIII to display a 50 amino acid sequence comprised of an LPETG (SEQ ID NO: 10) recognition motif for SrtAₐᵤᵣₑᵤₛ flanked by two cysteines. When these cysteines engage in disulfide bond formation, they form a loop similar to that displayed by the subunit A of cholera toxin.¹⁷ Because proteolytic cleavage of the loop improves labeling efficiency,¹⁷ we inserted a linker followed by a Factor Xa protease cleavage site immediately downstream of the LPETG (SEQ ID NO: 10) motif (FIG. 12a). We confirmed that sortase, pIII, pIX, and pVIII remained intact upon incubation with Factor Xa (data not shown). Thus, only the engineered pIII is a substrate for Factor Xa. This phage construct will be referred to hereafter as loopXa-pIII.

C-Terminal Sortase-Mediated Labeling of pIII.

We labeled the loopXa-pIII phage construct at pIII with a GGGK(TAMRA) peptide (SEQ ID NO: 127) using SrtAₐᵤᵣₑᵤₛ (FIG. 12b). Factor Xa was included in the reaction. We analyzed the samples by SDS-PAGE under both reducing and non-reducing conditions, followed by fluorescent imaging, and immunoblotting with an anti-pIII antibody. Only under non-reducing conditions and when all four reaction components were present did we observe a 60 kDa fluorescent anti-pIII reactive protein (FIG. 12b), consistent with the presence of an intramolecular disulfide bond and loop formation on a single pIII molecule.

Sortase-mediated transpeptidation reactions afford attachment of a wide range of molecules to this loop structure, including a pre-assembled protein complex of ˜58 kDa (FIG. 16). Of note, all the (poly)peptides conjugated in this fashion will display an exposed C-terminus.

To determine whether the loop engineered onto pIII renders itself suitable for labeling with larger molecules, we attempted to attach an oligomeric protein complex: the B subunit pentamer of cholera toxin (CtxB). CtxB represents a 58 kDa soluble complex (Zhang, R. G.; Westbrook, M. L.; Westbrook, E. M.; Scott, D. L.; Otwinowski, Z.; Maulik, P. R.; Reed, R. A.; Shipley, G. G., The 2.4 A crystal structure of cholera toxin B subunit pentamer: choleragenoid. J Mol Biol 1995, 251 (4), 550-62), which is disrupted by SDS at high temperatures. We endowed each single subunit of CtxB with three consecutive Gly residues at the N-terminus, expressed it in E. coli and purified the established pentamer (G₃-CtxB) (Antos, J. M.; Chew, G. L.; Guimaraes, C. P.; Yoder, N. C.; Grotenbreg, G. M.; Popp, M. W.; Ploegh, H. L., Site-specific N- and C-terminal labeling of a single polypeptide using sortases of different specificity. J Am Chem Soc 2009, 131 (31), 10800-1). Upon incubation of the LoopXa-pIII phage with Factor Xa, SrtAₐᵤᵣₑᵤₛ, and G₃-CtxB for 5 hrs at room temperature, the samples were boiled and analyzed by SDS-PAGE under non-reducing conditions, followed by immunoblot with anti-pIII and anti-CtxB antibodies (FIG. 16). Consistent with the size of the pIII-CtxB fusion, we detected a 75 kDa anti-pIII and anti-CtxB reactive protein only when all the reaction constituents are admixed. The identity of this protein was confirmed by mass-spectrometry (FIG. 16).

Orthogonal Labeling of Three Phage Capsid Proteins.

In a first attempt to establish end-to-end phage dimers, we tried to directly link the loopXa-pIII phage and a phage containing a pentaglycine motif at the N-terminus of its pIII (G₅-pIII phage) (SEQ ID NO: 77) via SrtAₐᵤᵣₑᵤₛ. No dimers were observed after 24 hrs of incubation and only ˜3% of structures were dimeric after 60 hrs of incubation (FIG. 17).

We attempted to directly fuse two phage particles through their ends using SrtAₐᵤᵣₑᵤₛ. One of the phage constructs contained a pentaglycine nucleophile motif (G₅-pIII phage) (SEQ ID NO: 77) and the other the loop structure (loopXa-pIII phage), both on pIII. 120 nM loopXa-pIII phage, 180 nM G₅-pIII phage (SEQ ID NO: 77), 230 nM Factor Xa, 30 μM SrtAₐᵤᵣₑᵤₛ, and 10 mM CaCl₂ in TBS were incubated at room temperature. Aliquots were taken at 24 hrs (no phage dimers were observed) and 60 hrs. The reaction was diluted with TBS, such that the loopXa-pIII concentration was below 10 nM, and purified by PEG8000/NaCl precipitation. Phage was resuspended in water and diluted to a concentration of 2·10¹¹ pfu/mL and imaged by atomic force microscopy (AFM) (FIG. 17). Dimer structures of roughly 2 μm in length were detected in ˜3% of the observed phage structures.

Given the slow kinetics of direct phage-phage fusion using SrtAₐᵤᵣₑᵤₛ, we hypothesized that the loopXa and pentaglycine motifs on phage could be individually labeled with oligoglycine or LPXTG-based (SEQ ID NO: 78) peptides before phage-phage fusions occur. With the ability to label pVIII orthogonally with SrtA_{pyogenes}, we created a phage construct (hereafter referred to as triSrt) containing three sortaggable motifs: loopXa on pIII, (A)₂ on pVIII, and G₅HA (SEQ ID NO: 77) on pIX (all at the N-terminus of the respective proteins). This combination enables selective labeling of three proteins on the same phage particle. The HA tag was added to pIX to extend its N-terminus and allow identification of the protein by immunoblots, as no antibodies are commercially available for pIX. We labeled each of these proteins in the triSrt construct with different fluorescent molecules (FIG. 13a) in a stepwise manner. First, pVIII was labeled with K(TAMRA)-LPETAA (SEQ ID NO: 12) using SrtA_{pyogenes} with subsequent purification of the desired reaction product by PEG8000/NaCl precipitation. The resultant TAMRA-pVIII phage was then incubated with SrtAₐᵤᵣₑᵤₛ, GGGK-Alexa647 (SEQ ID NO: 127), K(FAM)-LPETGG (SEQ ID NO: 13), and Factor Xa for 5 hrs at room temperature followed by PEG8000/NaCl precipitation. This precipitation allows purification of the labeled virions away from the other reaction components, including the side reaction product K(FAM)-LPETGGGK-Alexa647 (SEQ ID NO: 281) resultant from sortase-mediated fusion of the individual fluorescent peptides. Each reaction was analyzed by SDS-PAGE under non-reducing conditions followed by fluorescent imaging and immunoblot using anti-pIII and anti-HA antibodies (FIG. 13b). In the final product, we observed a TAMRA fluorescent ˜10 kDa protein compatible with the molecular weight of pVIII, an Alexa647 fluorescent and anti-pIII reactive 60 kDa protein (FIG. 13b, lanes 4 and 6), plus a FAM-fluorescent and anti-HA (pIX) reactive ˜10 kDa protein (FIG. 13b, lanes 5 and 6).

Labeling of pIII and pIX with DNA.

Because we can now functionalize the ends of the same phage particle orthogonally with different molecules, we sought to form phage trimers by DNA hybridization (FIG. 14a). Thiolated and Cy5-labeled DNA oligonucleotides were conjugated to either a (maleimide)-LPETGG (SEQ ID NO: 13) or GGGK(maleimide) peptides (Table 5) (SEQ ID NO: 127). The resultant DNA-peptide adducts were purified by size exclusion chromatography and analyzed by MALDI-TOF mass-spectrometry. The product displayed a size consistent with (maleimide)-LPETGG (SEQ ID NO: 13) (˜700 Da) and GGGK(maleimide) (SEQ ID NO: 127) (˜400 Da) peptides fused to the DNA oligonucleotides (FIG. 18a). These were also analyzed by TBE-Urea PAGE followed by fluorescent imaging (FIG. 18b). Upon reaction with maleimide-peptides, we observed a shift in mobility of the DNA, and did not detect any unreacted DNA, suggesting that all DNA was conjugated to the peptide.

Using SrtAₐᵤᵣₑᵤₛ and the triSrt phage, we attached DNA-peptides to pIII and to pIX forming three different phage constructs: DNA A-pIX phage, DNA B-pIII-DNA D-pIX phage, and DNA E-pIII phage (FIG. 14a). The reaction products were purified by PEG8000/NaCl precipitation. Free DNA-peptide co-precipitated with the phage, so an additional dialysis step was performed to remove it. The purified DNA-labeled phage was analyzed by SDS-PAGE under non-reducing conditions, followed by fluorescent imaging (FIG. 14b). Labeling of pIX with DNA A and DNA D (FIG. 14b, left panel) resulted in detection of Cy5-fluorescent 19 kDa and 22 kDa proteins, respectively. This is consistent with the predicted size of the DNA-pIX species. When pIII was labeled with DNA B and DNA E (FIG. 14b, right panel), we detected Cy5-fluorescent 75 kDa and 80 kDa proteins, respectively. These sizes are consistent with those expected for the DNA-pIII species.

Formation of Ordered Phage Trimers.

We mixed equimolar amounts of the above DNA-labeled virions, followed by addition of the hybridizing oligonucleotides DNA C and DNA F in 10-fold excess over phage (Table 5 and FIG. 14a). The mixture was heated at 95° C. and cooled to 20° C., thus allowing DNA to anneal and connect the phage particles. Atomic force microscopy (AFM) showed that this heating and cooling did not disrupt the integrity of the phage structure. Analysis of the annealed phage structure by AFM showed the existence of multi-phage structures of 2-3 μm in length (FIG. 14c and FIG. 19). No structures corresponding to phage particles intersecting with more than one phage at its end were detected, suggesting that the connections were indeed 1:1. We analyzed the phage population compiling a histogram of the lengths of observed structures (FIG. 14d and FIG. 19). For each treatment, at least 50 structures were measured. The length of a single phage is ˜880 nm. We thus assume that a structure <1 μm represents a single phage, 1-2 μm is two connected phage, 2-3 μm is three connected phage, and >3 μm is more than three connected phage. We observed that 52% of phage structures were 2-3 μm. Structures longer than 3 μm were observed rarely (5.8%), the longest observed structure being 4.70 μm. In contrast, when DNA C and DNA F were omitted from the reaction, 95% of the observed phage structures were less than 1 μm and no 2-3 μm structures could be found. Dynamic light scattering (DLS) showed an increase in the distribution of the particle sizes. When DNA C and DNA F were absent, we observed a peak for objects with a radius of ˜100 nm, corresponding to phage monomers. The size of the particles in the main peak increased significantly (˜1300 nm) when DNA C and DNA F were added. Particles comprising this peak were compatible with trimer structures based on the structures observed by AFM (FIG. 14d). Because phage is filamentous and not spherical, the numerical value of the hydrodynamic radii is reported to demonstrate only relative changes in size.

To confirm that the observed multi-phage structures were indeed formed by DNA hybridization, we incubated them with restriction enzymes: AatII cleaves the annealed DNA structure between DNA A-C, AgeI cleaves the connections between DNA D-F (FIG. 14a). The samples were analyzed using AFM and DLS (FIG. 14d and FIG. 20). Upon digestion with the individual enzymes, we observed a decrease in the structure length of the 2-3 μm phage particles (12% for AatII, 3.3% for AgeI), with structures of 1-2 μm in length being the most prevalent (46% for AatII, 62% for AgeI). This shift was consistent with the size distribution observed by DLS, where the peak for both AatII and AgeI digest shifted to ˜500 nm, corresponding to dimer phage structures. When the multi-phage preparation was incubated with both enzymes, we no longer observed phage structures of 2-3 μm in length and the majority of the population was under 1 μm (67%) (FIG. 14d and FIG. 20). These results were supported by DLS, where the peak of particle sizes decreased to ˜200 nm. We speculate that not all phage particles return to the monomeric form for reasons of steric hindrance: the phage structures themselves shielded the hybridized DNA from the restriction enzymes.

To ensure that the multi-phage structures were connected in the desired order, we fluorescently labeled the pVIII of the triSrt phage using SrtA_{pyogenes} with different fluorophores⁷, followed by DNA labeling. This yielded the following phage particles: TAMRA-pVIII-DNA A-pIX, DNA B-pIII-FAM-pVIII-DNA D-pIX, and DNA E-pIII-Alexa647-pVIII. We mixed these phage in equimolar amounts with a 10-fold excess DNA C and F, and imaged them by fluorescence microscopy (FIG. 14e and FIG. 21). We observed multi-color filamentous structures connected in the expected order: TAMRA, FAM and Alexa647 (FIG. 14a, FIG. 14e and FIG. 21). In the absence of DNA, such connected multi-color filamentous structures were not observed and only single-colored filaments were present (FIG. 21).

### CONCLUSIONS

Here we expand sortase-mediated labeling of M13 bacteriophage by engineering a loop onto pIII to enable C-terminal labeling. The insertion of a cleavable loop allows C-terminal exposure of the sortase motif LPXTG (SEQ ID NO: 78), and thus enables attachment of a substituted peptide or protein at that site via exposed Gly residues. Using this new structure, we attach a fluorophore and an oligomeric complex protein, neither of which could ever be displayed on the phage capsid genetically. Engineering of this loop onto pIII enables labeling orthogonal to the previously established N-terminal labeling method.^{7,18} Thus, we created a new phage construct with the loop structure on pIII, a pentaglycine motif on pIX, and a double alanine motif on pVIII. Although this configuration should theoretically allow direct phage to phage conjugation, we found this to be an inefficient reaction, possibly for steric reasons, and therefore resorted to the use of complementary DNA crossbridges to achieve our goal. We demonstrated as a proof of concept that the minor capsid proteins of phage can be labeled with DNA and used to form specific connections between different phage particles. This reaction was more efficient, with over 50% of observed phage structures displaying the length of trimers. The precision of this strategy surpasses earlier accomplishments in which phage were linked using leucine zippers: heterodisperse multi-phage structures were obtained with mean lengths of 3-4.5 μm (6-8 phage) and variability of length from monomers to longer than 20 phage.⁹

The DNA modified phage as a scaffold building block not only allows better control over the structures that can be produced, but this strategy should be readily extendable to create much longer multimers by the proper choice of different DNA sequences. Our work sets the stage for building more complex multi-phage structures, such as multi-way junctions,¹⁹ or combinations with DNA origami structures¹⁰ with the potential to control positions in three dimensions.²⁰ Attached DNA may also be used as a functional material sensitive to the environment such as pH,²¹ or bind substrates through the use of DNA aptamers,²²⁻²³ which extend the properties of the proteins or peptides displayed on the phage capsid, which has potential in biosensing applications.²⁴

The specific connection of phage particles, which we demonstrate, provides control of interactions between multiple materials at the nanoscale. Although the phage particles connected in this work were identical genetically, we attached different fluorophores to their pVIII body protein to establish that the requisite linkages were being formed in a pre-determined order. In principle, the ability to pattern phage with different pVIII proteins enables self-assembly of junctions between materials and formation of multi-material axial nanowires or even circuits. This ability potentially allows for phage-based devices where configuration and the proximity of materials are critical including transistor- and diode-based electronic devices.²⁵⁻²⁶

### Methods

Phage Engineering.

The oligonucleotides used in engineering phage are shown in Table S2. LoopXa-pIII phage was constructed from an M13KE vector (New England Biolabs). The vector was digested with Acc65I and EagI. The annealed oligonucleotides pIIILoop-C and pIIILoop-NC were annealed and ligated into the digested vector. The Factor Xa recognition site was introduced by mutagenesis using the Quik II Site-Directed Mutagenesis kit (Stratagene) with oligonucleotides pIIILoopXaTop and pIIILoopXaBottom. The p9G5HA vector phage construct⁷ served as template for the creating the triSrt phage. The loop containing the Factor Xa recognition site was installed on pIII as described above. Two alanine codons were introduced at the 5′ end of pVIII using PstI and BamHI restriction enzymes and the annealed pVIII-AA-C and pVIII-AA-NC oligonucleotides. The phage constructs were transformed, plated, and amplified as described.⁷

Sortase-Mediated Reactions.

Sortase reactions were performed as indicated in the figures. A typical sortase reaction for labeling LoopXa-pIII phage consisted of 160 nM phage, 30 μM SrtAₐᵤᵣₑᵤₛ, 230 nM Factor Xa, 100 μM GGGK(TAMRA) (SEQ ID NO: 127) or G₃ fused to the N-terminus of the B subunit of cholera toxin (G₃-CtxB), and 10 mM CaCl₂ in TBS (25 mM Tris, pH 7.0-7.4, and 150 mM NaCl) incubated for 5 hrs at room temperature. The concentration reported for G₃-CtxB is the monomer concentration. The sortase labeling reactions with GGGK(TAMRA) (SEQ ID NO: 127) were monitored by SDS-PAGE under reducing and non-reducing conditions followed by fluorescent imaging and immunoblot with an anti-pIII antibody (New England Biolabs). The CtxB labeling reactions were analyzed by SDS-PAGE in non-reducing conditions followed by immunoblot using an anti-pIII and anti-CtxB antibody (GenWay Biotech).

Typical conditions for labeling the pVIII of the triSrt phage were 160 nM phage, 40 μM SrtA_{pyogenes}, and 200 μM fluorophore conjugated LPETAA peptide (SEQ ID NO: 12) incubated for 3 hrs at room temperature followed by PEG8000/NaCl precipitation.⁷ The end labeling reactions of pIII and pIX consisted of 160 nM phage, 30 μM SrtAₐᵤᵣₑᵤₛ, 230 nM Factor Xa, and 100 μM of fluorescent peptide or 50 μM of DNA peptide in 10 mM CaCl₂ incubated for 5 hrs at room temperature followed by PEG8000/NaCl precipitation. For the DNA-phage reactions, additional purification was performed by dialysis against water with a 1 MDa molecular weight cut-off (Spectrum Labs), followed by another round of PEG8000/NaCl precipitation to purify and concentrate the samples.

DNA Peptide Conjugation.

The DNA oligonucleotides attached to the ends of phage are shown in Table 5. The thiol group on the DNA oligonucleotides was activated overnight with 0.1M DTT in PBS at 37° C. The DNA was then purified from excess DTT on a NAPS column (GE Healthcare) and eluted in water. The solution was dried and resuspended in PBS. (maleimide)-LPETGG (SEQ ID NO: 13) or GGGK(maleimide) (SEQ ID NO: 127) peptide in PBS was added in 2:1 molar excess of the activated DNA and reacted for 5 hrs at 37° C. In order to deactivate the excess maleimide, DTT was added to the mixture to give a concentration of 0.1M DTT and incubated at 37° C. for 15 min. The excess DTT and peptide was removed by purifying the reaction on a NAPS column. The DNA-peptide was dried under vacuum and resuspended in TBS. The concentration of the DNA-peptide was determined by UV-vis spectrometry using the absorbance at 260 nm. DNA-peptides were analyzed by a Micromass microMX MALDI with a pulsed 337 nm nitrogen laser. Spectra were acquired in positive ion, linear mode with a mass range of 2-30 kDa.

Atomic Force Microscopy and Dynamic Light Scattering.

The three DNA labeled phage were mixed together at 7.10¹³ pfu/mL in water. Hybridizing oligonucleotides DNA C and F were added in 10-fold molar excess. The reactions were heated to 95° C. for 5 minutes and cooled down to 20° C. at 0.5° C. per minute. For restriction enzyme digestion the phage were resuspended in NEB Buffer 4 (50 mM potassium acetate, 20 mM Tris-acetate, 10 mM magnesium acetate, 1 mM DTT, pH 7.9), and incubated at 37° C. for 3 hrs. We verified that the DTT in the NEB buffer did not disrupt the LoopXa-pIII structure by exposing LoopXa-pIII phage with Factor Xa to the buffer. We analyzed the reactions by SDS-PAGE followed by immunoblot with an anti-pIII antibody and estimated by densitometry that 10% of the LoopXa-pIII structures were disrupted, which represents only 1 pIII molecule for every two phage suggesting this did not significantly affect the connections.

To visualize the samples by AFM, phage preparations were diluted in water to a concentration of 2.10¹¹ pfu/mL. 90 μL of the phage solution was deposited on a freshly cleaved mica disc. AFM images were captured on a Nanoscope IV (Digital Instruments) in air using tapping mode. The tips had spring constants of 20-100N/m driven near their resonant frequency of 200-400 kHz (MikroMasch). The AFM images were analyzed and processed using Gwyddion. The histograms were collected by measuring the length of all phage events observed in seven 20 μm×20 μm areas.

DLS measurements were obtained with a DynaPro NanoStar (Wyatt Technology). Phage mixtures in NEB buffer 4 were diluted to 1·10¹³ pfu/mL in water. Samples from each experiment were measured 20 times and the results were averaged by cumulant analysis.

Fluorescence Microscopy.

The phage samples were diluted to 6·10¹¹ pfu/mL in water and 300 μL were deposited and dried on a glass cover slip. The samples were imaged using an inverted DeltaVision microscope equipped with an epifluorescent illumination module—488 nm laser (FAM—488 nm) and solid state illumination (TAMRA—543 nm and Alexa647), an oil immersion 100× objective (N.A.=1.40, 100×, Olympus) and Photometrics CoolSNAP HQ camera. All images were processed using ImageJ program (National Institutes of Health).

Miscellaneous.

Expression and purification of SrtA_{pyogenes}, SrtAₐᵤᵣₑᵤₛ and G₃-CtxB were performed as described.¹⁸ The LoopXa-pIII reactions were analyzed on 10% Laemmli SDS-PAGE gels. The pIX-DNA reactions were analyzed on a 16% Tricine-SDS PAGE gel, and the DNA-peptide conjugation reactions were analyzed on a 10% TBE-Urea PAGE gel (Life Technologies). All fluorescent gel images were collected on a Typhoon Trio (GE Healthcare). The GGGK(TAMRA) (SEQ ID NO: 127), K(FAM)-LPETGG (SEQ ID NO: 13), GGGK(maleimide) (SEQ ID NO: 127), (maleimide)-LPETGG (SEQ ID NO: 13), K(TAMRA)-LPETAA (SEQ ID NO: 279), and K(FAM)-LPETAA (SEQ ID NO: 12) peptides were obtained from the Swanson Biotechnology Center. For mass-spectrometry, the protein bands of interest were excised, subjected to protease digestion, and analyzed by electrospray ionization tandem mass-spectrometry (MS/MS).

TABLE 5
Sequences of the oligonucleotides used to link phage

| Name | Sequence (5′-3′) | Peptide |
|---|---|---|
| DNA A | Cy5-ACGTATCGTAGGCTCGCATCTTTTTTTTTT-SH | LPETGG |
| | (SEQ ID NO: 121) | (SEQ ID |
| | | NO: 13) |
| DNA B | HS-TTTTTTTTTTCTGCAGTTGAACCGGTAGCA-Cy5 | GGGK |
| | (SEQ ID NO: 122) | (SEQ ID |
| | | NO: 127) |
| DNA C | GAGCCTACGATACGTTGCTACCGGTTCAAC (SEQ ID NO: 123) | |
| DNA D | Cy5-GAGCGTGATTCGGATCCGTCATTCATCTACGCATCTTTTTTTTTT-SH | LPETGG |
| | (SEQ ID NO: 124) | (SEQ ID |
| | | NO: 13) |
| DNA E | HS-TTTTTTTTTTCTGCAGACGTCTTACCTCTAATCGATCGATCTCCG-Cy5 | GGGK |
| | (SEQ ID NO: 69) | (SEQ ID |
| | | NO: 127) |
| DNA F | GTAGATGAATGACGGATCCGAATCACGCTCCGGAGATCGATCGATTAG | |
| | AGGTAAGACGTC (SEQ ID NO: 125) | |

TABLE 6
Sequences of the oligonucleotides used for phage vector engineering

| | |
|---|---|
| Name | Sequence (5′-3′) |

LoopXa-pIII and triSrt engineering

| | |
|---|---|
| pIII-Loop-C | GTACCTTTCTATTCTCACTCTGAGCCGTGGATTCATCATGCACCGC |
| | CGGGTTGTGGGAATGCTCTTCCTGAGACCGGTGGTTACCCATACG |
| | ATGTTCCAGATTACGCTATGAATGCTCCAAGATCATCGATGAGTA |
| | ATACTTGCGATGAAAAAACCCAAAGTCTAGGTGTAAAAGGAGGC |
| | GGGTC (SEQ ID NO: 128) |
| pIII-Loop-NC | GGCCGACCCGCCTCCTTTTACACCTAGACTTTGGGTTTTTTCATCG |
| | CAAGTATTACTCATCGATGATCTTGGAGCATTCATAGCGTAATCT |
| | GGAACATCGTATGGGTAACCACCGGTCTCAGGAAGAGCATTCCCA |
| | CAACCCGGCGGTGCATGATGAATCCACGGCTCAGAGTGAGAATA |
| | GAAAG (SEQ ID NO: 129) |
| pIII-LoopXaTop | GTTCCAGATTACGCTATTGAAGGGAGATCATCGATGAATAC |
| | (SEQ ID NO: 130) |
| pIII-LoopXaBottom | GTATTCATCGATGATCTCCCTTCAATAGCGTAATCTGGAAC |
| | (SEQ ID NO: 131) |
| pVIII-AA-C | GCT TAT GAT ACG AAT ATG GAT TCG |
| | (SEQ ID NO: 132) |
| pVIII-AA-NC | GAT CCG AAT CCA TAT TCG TAT CAT AAG CTG CA |
| | (SEQ ID NO: 133) |

C-terminal phage vector display

| | |
|---|---|
| pIII-BspEIBclITop | CGTTTGCTAACATACTCCGGAATAAGGAGTCTTGATCATGCCAGT |
| | TCTTTTGG (SEQ ID NO: 134) |
| pIII-BspEIBclIBottom | CCAAAAGAACTGGCATGATCAAGACTCCTTATTCCGGAGTATGTT |
| | AGCAAACG (SEQ ID NO: 135) |
| pVI-AatIITop | AGGCTGCTATTTTCATTTTTGACGTCAAACAAAAAATCGTTTCTTA |
| | (SEQ ID NO: 136) |
| pVI-AatIIBottom | TAAGAAACGATTTTTTGTTTGACGTCAAAAATGAAAATAGCAGCC |
| | T (SEQ ID NO: 137) |
| pVI-AgeITop | ATATGGCTGTTTATTTTGTAACCGGTAAATTAGGCTCTGGAAAGA |
| | C (SEQ ID NO: 138) |
| pVI-AgeIBottom | GTCTTTCCAGAGCCTAATTTACCGGTTACAAAATAAACAGCCATA |
| | T (SEQ ID NO: 139) |
| pIX-SpeITop | TATTTTACCCGTTTAATGGAAACTAGTTCATGAAAAAGTCTTTAGT |
| | CC (SEQ ID NO: 140) |
| pIX-SpeIBottom | GGACTAAAGACTTTTTCATGAACTAGTTTCCATTAAACGGGTAAA |
| | ATA (SEQ ID NO: 141) |
| pIII-LPETGGHA-C | CCGGAATAAGGAGTCTCTACCGGAAACAGGAGGCTACCCATACG |
| | ATGTTCCAGATTACGCTT (SEQ ID NO: 142) |
| pIII-LPETGGHA-NC | GATCAAGCGTAATCTGGAACATCGTATGGGTAGCCTCCTGTTTCC |
| | GGTAGAGACTCCTTATT (SEQ ID NO: 143) |
| pIII-1GLPETGGHA-C | CCGGAATAAGGAGTCTGGAGGTGGAAGTCTACCGGAAACAGGAG |
| | GCTACCCATACGATGTTCCAGATTACGCTT (SEQ ID NO: 144) |
| pIII-1GLPETGGHA-NC | GATCAAGCGTAATCTGGAACATCGTATGGGTAGCCTCCTGTTTCC |
| | GGTAGACTTCCACCTCCAGACTCCTTATT (SEQ ID NO: 145) |
| pIII-3GLPETGGHA-C | CCGGAATAAGGAGTCTGGAGGTGGAAGTGGCGGTGGGAGCGGGG |
| | GAGGCTCTCTACCGGAAACAGGAGGCTACCCATACGATGTTCCAG |
| | ATTACGCTT (SEQ ID NO: 146) |
| pIII-3GLPETGGHA-NC | GATCAAGCGTAATCTGGAACATCGTATGGGTAGCCTCCTGTTTCC |
| | GGTAGAGAGCCTCCCCCGCTCCCACCGCCACTTCCACCTCCAGAC |
| | TCCTTATT (SEQ ID NO: 147) |
| pVI-LPETGGHA-C | CAAACAAAAAATCGTTTCTTATTTGGATTGGGATAAACTACCGGA |
| | AACAGGAGGCTACCCATACGACGTTCCAGATTACGCTTAATATGG |
| | CTGTTTATTTTGTAA (SEQ ID NO: 148) |
| pVI-LPETGGHA-NC | CCGGTTACAAAATAAACAGCCATATTAAGCGTAATCTGGAACGTC |
| | GTATGGGTAGCCTCCTGTTTCCGGTAGTTTATCCCAATCCAAATAA |
| | GAAACGATTTTTTGTTTGACGT (SEQ ID NO: 149) |
| pVI-1GLPETGGHA-C | CAAACAAAAAATCGTTTCTTATTTGGATTGGGATAAAGGAGGTGG |
| | AAGTCTACCGGAAACAGGAGGCTACCCATACGACGTTCCAGATTA |
| | CGCTTAATATGGCTGTTTATTTTGTAA (SEQ ID NO: 150) |
| pVI-1GLPETGGHA-NC | CCGGTTACAAAATAAACAGCCATATTAAGCGTAATCTGGAACGTC |
| | GTATGGGTAGCCTCCTGTTTCCGGTAGACTTCCACCTCCTTTATCC |
| | CAATCCAAATAAGAAACGATTTTTTGTTTGACGT (SEQ ID NO: 151) |
| pVI-3GLPETGGHA-C | CAAACAAAAAATCGTTTCTTATTTGGATTGGGATAAAGGAGGTGG |
| | AAGTGGCGGTGGGAGCGGGGGAGGCTCTCTACCGGAAACAGGAG |
| | GCTACCCATACGACGTTCCAGATTACGCTTAATATGGCTGTTTATT |
| | TTGTAA (SEQ ID NO: 152) |
| pVI3GLPETGGHA-NC | CCGGTTACAAAATAAACAGCCATATTAAGCGTAATCTGGAACGTC |
| | GTATGGGTAGCCTCCTGTTTCCGGTAGAGAGCCTCCCCCGCTCCC |
| | ACCGCCACTTCCACCTCCTTTATCCCAATCCAAATAAGAAACGAT |
| | TTTTTGTTTGACGT (SEQ ID NO: 153) |
| pIX-LPETGGHA-C | CTAGTTCTCTCCCGGAAACAGGTGGATACCCATACGATGTTCCAG |
| | ATTACGCTT (SEQ ID NO: 154) |
| pIX-LPETGGHA-NC | CATGAAGCGTAATCTGGAACATCGTATGGGTATCCACCTGTTTCC |
| | GGGAGAGAA (SEQ ID NO: 155) |
| pIX-1GLPETGGHA-C | CTAGTTCTGGAGGTGGAAGTCTCCCGGAAACAGGTGGATACCCAT |
| | ACGATGTTCCAGATTACGCTT (SEQ ID NO: 156) |
| pIX-1GLPETGGHA-NC | CATGAAGCGTAATCTGGAACATCGTATGGGTATCCACCTGTTTCC |
| | GGGAGACTTCCACCTCCAGAA (SEQ ID NO: 157) |
| pIX-3GLPETGGHA-C | CTAGTTCTGGAGGTGGAAGTGGCGGTGGGAGCGGGGGAGGCTCT |
| | CTCCCGGAAACAGGTGGATACCCATACGATGTTCCAGATTACGCT |
| | T (SEQ ID NO: 158) |
| pIX-3GLPETGGHA-NC | CATGAAGCGTAATCTGGAACATCGTATGGGTATCCACCTGTTTCC |
| | GGGAGAGAGCCTCCCCCGCTCCCACCGCCACTTCCACCTCCAGAA |
| | (SEQ ID NO: 159) |
| pIX-PCRprimer | CCCTCATAGTTAGCGTAACG (SEQ ID NO: 160) |
| pIIIpVI-PCRprimer | GTTGCTATTTTGCACCCAGC (SEQ ID NO: 161) |

### REFERENCES

(1) Sotiropoulou, S.; Siena-Sastre, Y.; Mark, S. S.; Batt, C. A., Biotemplated Nanostructured Materials. Chemistry of Materials 2008, 20 (3), 821-834.
(2) Nam, K. T.; Kim, D. W.; Yoo, P. J.; Chiang, C. Y.; Meethong, N.; Hammond, P. T.; Chiang, Y. M.; Belcher, A. M., Virus-enabled synthesis and assembly of nanowires for lithium ion battery electrodes. Science 2006, 312 (5775), 885-8.
(3) Lee, Y.; Kim, J.; Yun, D. S.; Nam, Y. S.; Shao-Horn, Y.; Belcher, A., Virus-templated Au and Au/Pt Core/Shell Nanowires and Their Electrocatalytic Activities for Fuel Cell Applications. Energy & Environmental Science 2012.
(4) Dang, X.; Yi, H.; Ham, M. H.; Qi, J.; Yun, D. S.; Ladewski, R.; Strano, M. S.; Hammond, P. T.; Belcher, A. M., Virus-templated self-assembled single-walled carbon nanotubes for highly efficient electron collection in photovoltaic devices. Nat Nanotechnol 2011, 6 (6), 377-84.
(5) Lee, Y. J.; Yi, H.; Kim, W. J.; Kang, K.; Yun, D. S.; Strano, M. S.; Ceder, G.; Belcher, A. M., Fabricating genetically engineered high-power lithium-ion batteries using multiple virus genes. Science 2009, 324 (5930), 1051-5.
(6) Huang, Y.; Chiang, C.-Y.; Lee, S. K.; Gao, Y.; Hu, E. L.; Yoreo, J. D.; Belcher, A. M., Programmable Assembly of Nanoarchitectures Using Genetically Engineered Viruses. Nano letters 2005, 5 (7), 1429-1434.
(7) Hess, G. T.; Cragnolini, J. J.; Popp, M. W.; Allen, M. A.; Dougan, S. K.; Spooner, E.; Ploegh, H. L.; Belcher, A. M.; Guimaraes, C. P., M13 bacteriophage display framework that allows sortase-mediated modification of surface-accessible phage proteins. Bioconjug Chem 2012, 23 (7), 1478-87.
(8) Nam, K. T.; Peelle, B. R.; Lee, S.-W.; Belcher, A. M., Genetically Driven Assembly of Nanorings Based on the M13 Virus. Nano letters 2003, 4 (1), 23-27.
(9) Sweeney, R. Y.; Park, E. Y.; Iverson, B. L.; Georgiou, G., Assembly of multimeric phage nanostructures through leucine zipper interactions. Biotechnology and bioengineering 2006, 95 (3), 539-545.
(10) Stephanopoulos, N.; Liu, M.; Tong, G. J.; Li, Z.; Liu, Y.; Yan, H.; Francis, M. B., Immobilization and one-dimensional arrangement of virus capsids with nanoscale precision using DNA origami. Nano letters 2010, 10 (7), 2714-2720.
(11) Cigler, P.; Lytton-Jean, A. K. R.; Anderson, D. G.; Finn, M.; Park, S. Y., DNA-controlled assembly of a NaT1 lattice structure from gold nanoparticles and protein nanoparticles. Nature materials 2010, 9 (11), 918-922.
(12) Park, S. Y.; Lytton-Jean, A. K. R.; Lee, B.; Weigand, S.; Schatz, G. C.; Mirkin, C. A., DNA-programmable nanoparticle crystallization. Nature 2008, 451 (7178), 553-556.
(13) Nykypanchuk, D.; Maye, M. M.; van der Lelie, D.; Gang, O., DNA-guided crystallization of colloidal nanoparticles. Nature 2008, 451 (7178), 549-552.
(14) Xiang, D.-s.; Zeng, G.-p.; He, Z.-k., Magnetic microparticle-based multiplexed DNA detection with biobarcoded quantum dot probes. Biosensors and Bioelectronics 2011, 26 (11), 4405-4410.
(15) Goldmann, A. S.; Barner, L.; Kaupp, M.; Vogt, A. P.; Barner-Kowollik, C., Orthogonal ligation to spherical polymeric microparticles: Modular approaches for surface tailoring. Progress in Polymer Science 2012, 37 (7), 975-984.
(16) Race, P. R.; Bentley, M. L.; Melvin, J. A.; Crow, A.; Hughes, R. K.; Smith, W. D.; Sessions, R. B.; Kehoe, M. A.; McCafferty, D. G.; Banfield, M. J., Crystal structure of Streptococcus pyogenes sortase A: implications for sortase mechanism. J Biol Chem 2009, 284 (11), 6924-33.
(17) Guimaraes, C. P.; Carette, J. E.; Varadarajan, M.; Antos, J.; Popp, M. W.; Spooner, E.; Brummelkamp, T. R.; Ploegh, H. L., Identification of host cell factors required for intoxication through use of modified cholera toxin. J Cell Biol 2011, 195 (5), 751-64.
(18) Antos, J. M.; Chew, G. L.; Guimaraes, C. P.; Yoder, N. C.; Grotenbreg, G. M.; Popp, M. W.; Ploegh, H. L., Site-specific N- and C-terminal labeling of a single polypeptide using sortases of different specificity. J Am Chem Soc 2009, 131 (31), 10800-1.
(19) Cheng, E.; Xing, Y.; Chen, P.; Yang, Y.; Sun, Y.; Zhou, D.; Xu, L.; Fan, Q.; Liu, D., A pH-Triggered, Fast-Responding DNA Hydrogel. Angewandte Chemie International Edition 2009, 48 (41), 7660-7663.
(20) Ke, Y.; Ong, L. L.; Shih, W. M.; Yin, P., Three-dimensional structures self-assembled from DNA bricks. Science 2012, 338 (6111), 1177-83.
(21) Modi, S.; Swetha, M.; Goswami, D.; Gupta, G. D.; Mayor, S.; Krishnan, Y., A DNA nanomachine that maps spatial and temporal pH changes inside living cells. Nat Nanotechnol

2009, 4 (5), 325-330.
(22) Ellington, A. D.; Szostak, J. W., Selection in vitro of single-stranded DNA molecules that fold into specific ligand-binding structures. Nature 1992, 355 (6363), 850-852.
(23) Song, S.; Wang, L.; Li, J.; Fan, C.; Zhao, J., Aptamer-based biosensors. TrAC Trends in Analytical Chemistry 2008, 27 (2), 108-117.
(24) Lee, J. H.; Domaille, D. W.; Cha, J. N., Amplified Protein Detection and Identification through DNA-Conjugated M13 Bacteriophage. ACS Nano 2012, 6 (6), 5621-5626.
(25) Kempa, T. J.; Tian, B.; Kim, D. R.; Hu, J.; Zheng, X.; Lieber, C. M., Single and tandem axial pin nanowire photovoltaic devices. Nano letters 2008, 8 (10), 3456-3460.
(26) Cui, Y.; Lieber, C. M., Functional nanoscale electronic devices assembled using silicon nanowire building blocks. Science 2001, 291 (5505), 851-853.

All publications, patents, patent applications, and database entries mentioned anywhere herein, including, but not limited to, those items listed above, are hereby incorporated by reference in their entirety as if each individual publication, patent, patent application, and database entry was specifically and individually indicated to be incorporated by reference. In case of conflict, the present application, including any definitions herein, will control.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

1. A method of modifying a target protein comprising a sortase recognition motif on the surface of a virus, the method comprising
contacting the target protein with a sortase substrate conjugated to an agent in the presence of a sortase under conditions suitable for the sortase to conjugate the target protein and the sortase substrate.

2. The method of claim 1, wherein the target protein comprises an N-terminal sortase recognition motif.

3. The method of claim 2, wherein the N-terminal sortase recognition motif comprises an oligoglycine or an oligoalanine sequence.

4. The method of claim 3, wherein the oligoglycine and/or the oligoalanine comprises 1-10 N-terminal glycine residues or 1-10 N-terminal alanine residues, respectively.

5. The method of claim 1, wherein the sortase substrate comprises a C-terminal sortase recognition motif.

6. The method of claim 5, wherein the C-terminal recognition motif is LPXTX, wherein each instance of X independently represents any amino acid residue.

7. The method of claim 6, wherein the C-terminal recognition motif is LPETG (SEQ ID NO: 10) or LPETA (SEQ ID NO: 11).

8. The method of claim 1, wherein the sortase is sortase A from Staphylococcus aureus (SrtAₐᵤᵣₑᵤₛ) or sortase A from Streptococcus pyogenes (SrtA_{pyogenes}).

9. The method of claim 1, wherein the virus is a DNA virus.

10. The method of claim 1, wherein the virus is a bacteriophage.

11. The method of claim 10, wherein the virus is an M13 bacteriophage.

12. The method of claim 1, wherein the target protein is a viral capsid protein.

13. The method of claim 12, wherein the target protein is M13 pIII, pVIII, or PIX.

14. The method of claim 1, wherein the agent is a protein, a lipid, a carbohydrate, a nucleic acid, a detectable label, a binding agent, a click-chemistry handle, or a small molecule.

15. The method of claim 14, wherein the agent is a fluorescent protein, streptavidin, biotin, a fluorophore, an antibody or an antibody fragment, a bacterial toxin, a plant toxin, an enzyme, a multi-protein complex, an alkyne, an azide, a diene, a dienophile, a thiol, an alkene, an aryne, a tetrazine, a tetrazole, a dithioester, an anthracene, a maleimide, an enone, or an amine.

16. The method of claim 1, wherein the method comprises multiple rounds of modifying a target protein on the surface of the same virus, and wherein a different target protein is modified in each round.

17. The method of claim 16, wherein at least one of the target proteins is modified using SrtAₐᵤᵣₑᵤₛ, and at least one other target protein is modified using SrtA_{pyogenes}.

18. The method of claim 16, wherein a different agent is conjugated to each target protein.

19. A virus comprising a target protein that has been modified by the method of claim 1.

20. A method of associating viral particles, the method comprising
(a) conjugating a first target protein on the surface of the viral particle with a first binding agent via sortase-mediated transpeptidation;
(b) conjugating a second target protein on the surface of the viral particle with a second binding agent, wherein the second binding agent binds the first binding agent; and
(c) incubating a plurality of viral particles of steps (a) and (b) under conditions suitable for the first and the second binding agent of different viral particles to bind each other.

21.-35. (canceled)

36. A virus comprising a target protein that is conjugated to an agent via a sortase recognition motif.

37.-52. (canceled)

53. A virus comprising a recombinant target protein, wherein the recombinant target protein comprises a sortase recognition motif.

54.-74. (canceled)
